(19) United States
(12) Patent Application Publication
(10) Pub. No.: US 2004/0171614 A1
(43) Pub. Date: Sep. 2, 2004

(54) Novel gamma secretase inhibitors

(75) Inventors: Dmitri A. Pissarnitski, Scotch Plains, NJ (US); Hubert B. Josien, Hoboken, NJ (US); Elizabeth M. Smith, Verona, NJ (US); John W. Clader, Cranford, NJ (US); Theodros Asberom, West Orange, NJ (US); Tao Guo, Dayton, NJ (US); Douglas W. Hobbs, Yardley, PA (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignees: Schering-Plough Corporation; Pharmacopeia, Inc.

(21) Appl. No.: 10/663,042

(22) Filed: Sep. 16, 2003

Related U.S. Application Data
(63) Continuation-in-part of application No. 10/358,898, filed on Feb. 5, 2003.
(60) Provisional application No. 60/355,618, filed on Feb. 6, 2002.

(51) Int. Cl. A61K 31/5377; A61K 31/496; A61K 31/4545; A61K 31/454; C07D 413/02; C 07D 4 3/02

(52) U.S. Cl. 514/235.2; 514/253.01; 514/316; 514/326; 544/129; 544/360; 546/186; 546/208

(57) ABSTRACT

This invention discloses novel gamma secretase inhibitors of the formula:

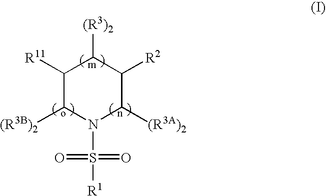

wherein:

R¹ is a substituted aryl or substituted heteroaryl group;

R² is an R¹ group, alkyl, —XC(O)Y, alkylene-XC(O)Y, cycloalkylene-X-C(O)—Y, —CH—X—C(O)—NR³—Y or —CH—X—C(O)—Y, wherein X and Y are as defined herein;

each R³ and each R^{3A} are independently H, or alkyl;

R¹¹ is aryl, heteroaryl, alkyl, cycloalkyl, arylalkyl, arylcycloalkyl, heteroarylalkyl, heteroarylcycloalkyl, arylheterocycloalkyl, or alkoxyalkyl. Also disclosed is a method of treating Alzheimer's Disease using one or more compounds of the invention.

This application is a continuation-in-part of patent application, Ser. No. 10/358,898 filed on Feb. 5, 2003, which claims priority from provisional application Serial No. 60/355,618 filed Feb. 6, 2002.

### BACKGROUND OF THE INVENTION

WO 00/50391, published Aug. 13, 2000, discloses compounds having a sulfonamide moiety that are useful for the treatment and prevention of Alzheimer's Disease and other diseases relating to the deposition of amyloid protein.

In view of the present interest in the treatment or prevention of neurodegenerative diseases, such as Alzheimer's Disease, a welcome contribution to the art would be compounds for use in such treatment or prevention. This invention provides such a contribution.

### SUMMARY OF THE INVENTION

This invention provides compounds that are inhibitors (e.g., antagonists) of Gamma Secretase and have the formula:

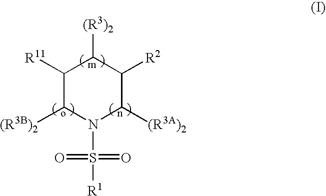

or a pharmaceutically acceptable salt, solvate or ester thereof, wherein:

wherein:

(A) R¹ is selected from the group consisting of:

(1) unsubstituted aryl;

(2) aryl substituted with one or more R⁵ groups;

(3) unsubstituted heteroaryl; and

(4) heteroaryl substituted with one or more R⁵ groups,

(B) R is selected from the group consisting of:

(1) alkyl;

(2) —XC(O)Y;

(3) —(C₁-C₆)alkylene-XC(O)Y;

(4) —(C₀-C₆)alkylene-(C₃-C₆)cycloalkylene-(C₀-C₆)alkylene-XC(O)Y;

(5) aryl;

(6) aryl substituted with one or more R⁵ groups;

(7) heteroaryl;

(8) heteroaryl substituted with one or more R⁵ groups;

(9) cycloalkylene-X-C(O)—Y;

(10 ) —CH₂—X—C(O)—NR³—Y;

(11) —CH₂—X—C(O)—Y; and

(12) —CH₂—X—C(O)—NR³—Y,

(C) Each R³ is independently selected from the group consisting of:

(1) H; and

(2) alkyl,

(D) Each R^{3A} and R^{3B} is independently selected from the group consisting of:

(1) H; and

(2) alkyl;

(E) R⁵ is independently selected from the group consisting of:

(1) halo;

(2) —CF₃;

(3) —OH;

(4) —O-alkyl;

(5) —OCF₃;

(6) —CN;

(7) —NH₂;

(8) —C(O)₂alkyl;

(9) —C(O)NR⁶R⁷;

(10) -alkylene-NR⁶R⁷;

(11) —NR⁶C(O)alkyl;

(12) —NR⁶C(O)aryl;

(13) —NR⁶C(O)heteroaryl; and

(14) —NR 6C(O)NR⁶R⁷;

(F) X is selected from the group consisting of:

(1) —O—;

(2) —NH—;

(3) —N-alkyl; and

(4) —O-alkylene;

(G) Y is selected from the group consisting of:

(1) —NR⁶R⁷;

(2) —N(R³)(CH₂)_{b}NR⁶R⁷ wherein b is 2-6;

(3) unsubstituted aryl;

(4) unsubstituted heteroaryl;

(5) -alkyl;

(6) -cycloalkyl,

(7) unsubstituted arylalkyl;

(8) unsubstituted arylcycloalkyl;

(9) unsubstituted heteroarylalkyl;

(10) unsubstituted heteroarylcycloalkyl;

(11) unsubstituted arylheterocycloalkyl;

(12) substituted aryl;

(13) substituted heteroaryl;

(14) substituted arylalkyl;

(15) substituted arylcycloalkyl;

(16) substituted heteroarylalkyl;

(17) substituted heteroarylcycloalkyl; and

(18) substituted arylheterocycloalkyl;

(19) substituted heterocycloalkyl alkyl;

(20) unsubstituted heteroaryl alkyl;

(21) unsubstituted aryl alkyl heterocycloalkyl;

(22) unsubstituted heterocycloalkyl; and

(23) unsubstituted cycloalkyl,

wherein the aryl moiety in said substituted groups (12), (14), (15), (18), and (21) of said Y group, and the heteroaryl moiety in said substituted groups (13), (16), (17) and (20) of said Y group, are substituted with one or more substituents independently selected from the group consisting of:

(a) halo;

(b) —CF₃;

(c) —OH;

(d) —O-alkyl;

(e) —OCF₃;

(f) —CN;

(g) —NH₂;

(h) —C(O)₂(C₁-C₆)alkyl;

(i) —C(O)NR⁶R⁷;

(j) —(C₁-C₆)alkylene-NR⁶R⁷;

(k) —NR⁶C(O)alkyl;

(l) —NR⁶C(O)aryl;

(m) —NR⁶C(O)heteroaryl;

(n) —NR⁶C(O)NR⁶R⁷; and

(o) alkyl,

or Y is selected from the group consisting of:

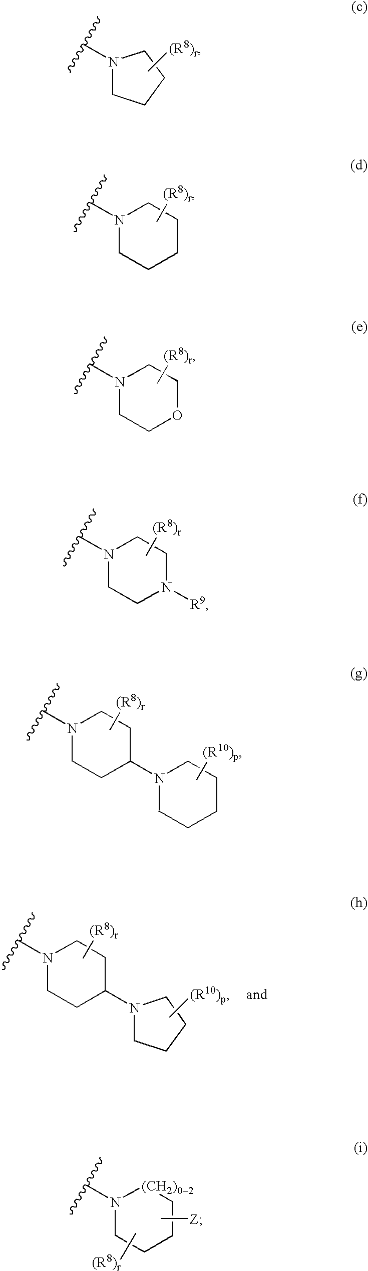

(H) R⁶ and R⁷ are independently selected from the group consisting of:

(1) H;

(2) alkyl;

(3) cycloalkyl;

(4) arylalkyl;

(5) heteroarylalkyl;

(6)

(7)

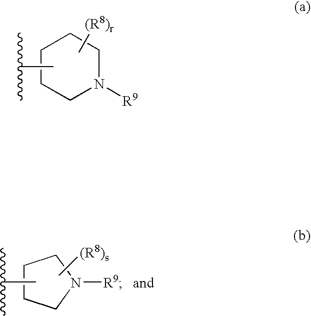

(8) heterocycloalkyl,

(I) Each R⁸ is independently selected from the group consisting of:

(1) alkyl;

(2) alkyl substituted with 1 to 4 hydroxy groups; and

(3) —OH,

(J) Each R⁹ is independently selected from the group consisting of:

(1) H;

(2) alkyl;

(3) alkyl substituted with 1 to 4 hydroxy groups;

(4) cycloalkyl;

(5) cycloalkyl substituted with 1 to 4 hydroxy groups;

(6) arylalkyl;

(7) heteroarylalkyl;

(8) —C(O)O-alkyl;

(9) alkylene-O-alkylene-OH;

(10) aryl substituted with one or more R⁵ groups;

(11) heteroaryl substituted with one or more R⁵ groups;

(12) unsubstituted heteroaryl;

(13) unsubstituted aryl;

(14) -alkylene-C(O)O-alkyl; and

(15) hydroxyalkyl-O-alkyl,

(K) Each R¹⁰ is independently selected from the group consisting of:

(1) H; and

(2) alkyl,

(L) R¹¹ is selected from the group consisting of:

(1) unsubstituted aryl;

(2) substituted aryl;

(3) unsubstituted heteroaryl,

(4) alkyl;

(5) cycloalkyl;

(6) unsubstituted arylalkyl;

(7) unsubstituted arylcycloalkyl,

(8) unsubstituted heteroarylalkyl;

(9) unsubstituted heteroarylcycloalkyl;

(10) unsubstituted arylheterocycloalkyl;

(11) alkoxyalkyl;

(12) substituted heteroaryl;

(13) substituted arylalkyl;

(14) substituted arylcycloalkyl;

(15) substituted heteroarylalkyl; and

(16) substituted arylheterocycloalkyl,

wherein the aryl moiety in said substituted groups (2), (13), (14) and (16) of said R¹¹ group, and the heteroaryl moiety in said substituted groups (12) and (15) of said R¹¹ group, are substituted with one or more substituents independently selected from the group consisting of:

(a) halo;

(b) —CF₃;

(c) —OH;

(d) —O-alkyl;

(e) —OCF₃;

(f) —CN;

(g) —NH₂;

(h) —C(O)₂(C₁-C₆)alkyl;

(i) —C(O)NR⁶R⁷;

(j) —(C₁-C₆)alkylene-NR⁶R⁷;

(k) —NR⁶C(O)alkyl;

(l) —NR⁶C(O)aryl;

(m) —NR⁶C(O)heteroaryl; and

(n) —NR⁶C(O)NR⁶R⁷;

(M) (1) m is 0 to 3, and if m is greater than 1, m moieties can be the same or different from one another;

(2) n is 0 to 3, and if n is greater than 1, n moieties can be the same or different from one another;

(3) o is 0 to 3, and if o is greater than 1, o moieties can be the same or different from one another; such that m+n+o is 1, 2, 3 or 4;

(N) p is 0 to 4, and if greater than 1, p moieties can be the same or different from one another;

(O) r is 0 to 4, and if greater than 1, r moieties can be the same or different from one another;

(P) s is 0 to 3, and if greater than 1, s moieties can be the same or different from one another; and

(Q) Z is selected from the group consisting of:

(1) unsubstituted heterocycloalkyl;

(2) substituted heterocycloalkyl;

(3) —NH₂;

(4) —NH(alkyl);

(5) —N(alkyl)₂ wherein each alkyl is the same or different;

(6) —NH(unsubstituted cycloalkyl);

(7) —NH(substituted cycloalkyl);

(8) —N(alkyl)(unsubstituted cycloalkyl);

(9) —N(alkyl)(substituted cycloalkyl);

(10) —NH(unsubstituted aralkyl);

(11) —NH(substituted aralkyl);

(12) —N(alkyl)(aralkyl);

(13) —NH(unsubstituted heterocycloalkyl);

(14) —NH(substituted heterocycloalkyl);

(15) —N(alkyl)(unsubstituted heterocycloalkyl),

(16) —N(alkyl)(substituted heterocycloalkyl);

(17) —NH(unsubstituted heteroaralkyl);

(18) —NH(substituted heteroaralkyl);

(19) —NH-alkylene-(unsubstituted cycloalkyl);

(20) —NH-alkylene-(substituted cycloalkyl);

(21) —N(alkyl)alkylene-(unsubstituted cycloalkyl);

(22) —N(alkyl)alkylene-(substituted cycloalkyl);

(23) —NHalkylene-(unsubstituted heterocycloalkyl);

(24) —NHalkylene-(substituted heterocycloalkyl);

(25) —N(alkyl)alkylene-(unsubstituted heterocycloalkyl);

(26) —N(alkyl)alkylene-(substituted heterocycloalkyl);

(27) unsubstituted benzofused heterocycloalkyl; and

(28) substituted benzofused heterocycloalkyl;

(29) H; and

(30) —N(hydroxyalkyl)₂, wherein each alkyl may be the same or different,

wherein said substituted heterocycloalkyl moiety of substituents (2), (14), (16), (24), (26) and (27) of group Z, and said substituted cycloalkyl moiety of substituents (7), (9), (20) and (22) of group Z, and said substituted aryl moiety of substituent (11) of group Z, and said substituted heteroaryl moiety of substituent (18) of group Z, are substituted with 1 to 3 groups independently selected from the group consisting of:

(a) alkyl;

(b) —OH;

(c) —Oalkyl;

(d) —OC(O)alkyl;

(e) —OC(O)aryl;

(f) —NH₂;

(g) —NH(alkyl);

(h) —N(alkyl)₂ wherein each alkyl is the same or different;

(i) —NHC(O)alkyl;

(j) —N(alkyl)C(O)alkyl;

(k) —NHC(O)aryl;

(l) —N(alkyl)C(O)aryl;

(m) —C(O)alkyl;

(n) —C(O)aryl;

(o) —C(O)NH₂;

(p) —C(O)NH(alkyl);

(q) —C(O)N(alkyl)₂ wherein each alkyl is the same or different;

(r) —C(O)₂alkyl;

(s) -alkylene-C(O)Oalkyl;

(t) piperidinyl;

(u) pyrrolidinyl;

(v) 1,1-ethylenedioxy;

(w) aryl;

(x) heteroaryl; and

(y) —O—CH₂CH₂—O-wherein both oxygen atoms are bound to the same carbon atom, and provided that the aryl and heteroaryl moieties of said Z group are not substituted with said —O—CH₂CH₂—O-group.

In (M) through (P), each reference to moieties preceded by an index, e.g., “m moieties”, refers to the moieties quantified by that index. Thus, for example, the term “m moieties” refers to the moieties whose quantity is indicated by the index “m”.

This invention further provides compounds that are inhibitors of Gamma Secretase selected from the group consisting of:

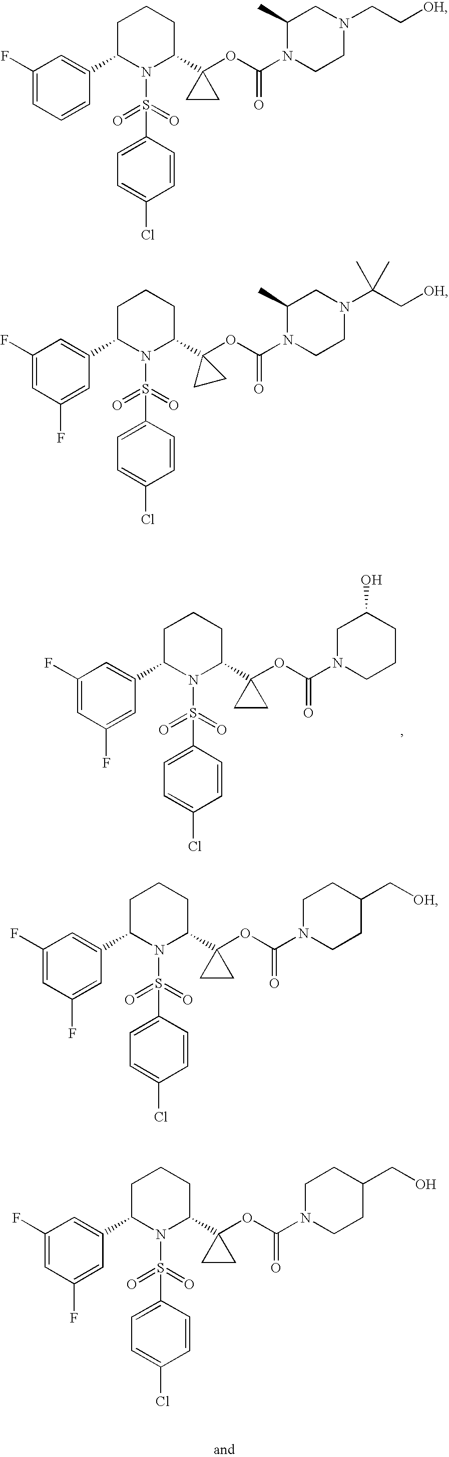

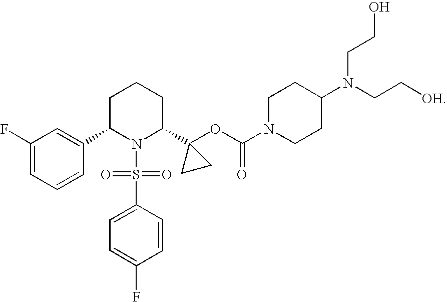

This invention also provides a pharmaceutical composition comprising an effective amount of one or more compounds of the above formulas and at least one pharmaceutically acceptable carrier.

This invention also provides a method for inhibiting gamma-secretase comprising administering an effective (i.e., therapeutically effective) amount of one or more compounds of the above formulas to a patient in need of such inhibition.

This invention also provides a method of treating one or more neurodegenerative diseases comprising administering an effective (i.e., therapeutically effective) amount of one or more compounds of the above formulas to a patient in need of treatment.

This invention also provides a method of inhibiting the deposition of amyloid protein (e.g., amyloid beta protein) in, on or around neurological tissue (e.g., the brain) comprising administering an effective (i.e., therapeutically effective) amount of one or more compounds of the above formulas to a patient in need of such inhibition.

This invention also provides a method of treating Alzheimer's disease comprising administering an effective (i.e., therapeutically effective) amount of one or more compounds of the above formulas to a patient in need of treatment.

### DETAILED DESCRIPTION OF THE INVENTION

As used above, and throughout the specification, the following terms, unless otherwise indicated, shall be understood to have the following meanings:

“Patient” includes both human and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The term “substituted alkyl” means that the alkyl group may be substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy, —C(O)O-alkyl and —S(alkyl). Non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. The term “substituted alkenyl” means that the alkenyl group may be substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl, 3-methylbutynyl, n-pentynyl, and decynyl. The term “substituted alkynyl” means that the alkynyl group may be substituted by one or more substituents which may be the same or different, each substituent being independently selected from the group consisting of alkyl. aryl and cycloalkyl.

“Alkylene” means a difunctional group obtained by removal of a hydrogen atom from an alkyl group that is defined above. Non-limiting examples of alkylene include methylene, ethylene and propylene.

“Aryl” (sometimes abbreviated “ar”) means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

“Aralkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting examples of suitable alkylaryl groups include o-tolyl, p-tolyl and xylyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalin, norbornyl, adamantyl and the like.

“Halo” means fluoro, chloro, bromo, or iodo groups. Preferred are fluoro, chloro or bromo, and more preferred are fluoro and chloro.

“Halogen” means fluorine, chlorine, bromine, or iodine. Preferred are fluorine, chlorine or bromine, and more preferred are fluorine and chlorine.

“Haloalkyl” means an alkyl as defined above wherein one or more hydrogen atoms on the alkyl is replaced by a halo group defined above.

“Ring system substituent” means a substituent attached to an aromatic or non-aromatic ring system which, for example, replaces an available hydrogen on the ring system. Ring system substituents may be the same or different, each being independently selected from the group consisting of alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-, Y₁Y₂NC(O)— and Y₁Y₂NSO₂—, wherein Y₁ and Y₂ may be the same or different and are independently selected from the group consisting of hydrogen, alkyl, aryl, and aralkyl. “Ring system substituent” also means a cyclic ring of 3 to 7 ring atoms of which 1-2 may be a heteroatom, attached to an aryl, heteroaryl, heterocyclyl or heterocyclenyl ring by simultaneously substituting two ring hydrogen atoms on said aryl, heteroaryl, heterocyclyl or heterocyclenyl ring. Non-limiting examples include:

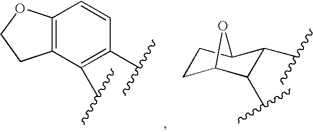

and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cycloheptenyl, and the like. Non-limiting example of a suitable multicyclic cycloalkenyl is norbornylenyl.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable monocyclic azaheterocyclenyl groups include 1,2,3,4-tetrahydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like. Non-limiting examples of suitable oxaheterocyclenyl groups include 3,4-dihydro-2H-pyran, dihydrofuranyl, fluorodihydrofuranyl, and the like. Non-limiting example of a suitable multicyclic oxaheterocyclenyl group is 7-oxabicyclo[2.2.1]heptenyl. Non-limiting examples of suitable monocyclic thiaheterocyclenyl rings include dihydrothiophenyl, dihydrothiopyranyl, and the like.

“Heterocyclyl” (or heterocycloalkyl) means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. The heterocyclyl can be optionally substituted by one or more “ring system substituents” which may be the same or different on the carbon(s) and/or heteroatoms(s), and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

“Arylcycloalkenyl” means a group derived from a fused aryl and cycloalkenyl as defined herein by removal of a hydrogen atom from the cycloalkenyl portion. Preferred arylcycloalkenyls are those wherein aryl is phenyl and the cycloalkenyl consists of about 5 to about 6 ring atoms. The arylcycloalkenyl can be optionally substituted by one or more ring system substituents, wherein “ring system substituent” is as defined above. Non-limiting examples of suitable arylcycloalkenyls include 1,2-dihydronaphthalene, indene, and the like. The bond to the parent moiety is through a non-aromatic carbon atom.

“Cycloalkenylaryl” means a group derived from a fused arylcycloalkenyl as defined herein by removal of hydrogen atom from the aryl portion. Non-limiting examples of suitable cycloalkenylaryls are as described herein for a arylcycloalkenyl, except that the bond to the parent moiety is through an aromatic carbon atom.

“Arylcycloalkyl” means a group derived from a fused aryl and cycloalkyl as defined herein by removal of a hydrogen atom from the cycloalkyl portion. Preferred arylcycloalkyls are those wherein aryl is phenyl and the cycloalkyl consists of about 5 to about 6 ring atoms. The arylcycloalkyl can be optionally substituted by one or more ring system substituents, wherein “ring system substituent” is as defined above. Non-limiting examples of suitable arylcycloalkyls include 1,2,3,4-tetrahydronaphthyl, and the like. The bond to the parent moiety is through a non-aromatic carbon atom.

“Cycloalkylaryl” means a group derived from a fused arylcycloalkyl as defined herein by removal of a hydrogen atom from the aryl portion. Non-limiting examples of suitable cycloalkylaryls are as described herein for an arylcycloalkyl group, except that the bond to the parent moiety is through an aromatic carbon atom.

“Heteroarylcycloalkyl” means a group derived from a fused heteroaryl and cycloalkyl as defined herein by removal of a hydrogen atom from the cycloalkyl portion. Preferred heteroarylcycloalkyls are those wherein the heteroaryl thereof consists of about 5 to about 6 ring atoms and the cycloalkyl consists of about 5 to about 6 ring atoms. The prefix aza, oxa or thia before heteroaryl means that at least a nitrogen, oxygen or sulfur atom is present respectively as a ring atom. The heteroarylcycloalkyl can be optionally substituted by one or more ring system substituents, wherein “ring system substituent” is as defined above. The nitrogen atom of the heteroaryl portion of the heteroarylcycloalkyl can be optionally oxidized to the corresponding N-oxide. Non-limiting examples of suitable heteroarylcycloalkyls include 5,6,7,8-tetrahydroquinolinyl, 5,6,7,8-tetrahydroisoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydrobenzoxazolyl, 1H-4-oxa-1,5-diazanaphthalen-2-onyl, 1,3-dihydroimidizole-[4,5]-pyridin-2-onyl, and the like. The bond to the parent moiety is through a non-aromatic carbon atom.

“Cycloalkylheteroaryl” means a group derived from a fused beteroarylcycloalkyl as defined herein by removal of a hydrogen atom from the heteroaryl portion. Non-limiting examples of suitable cycloalkylheteroaryls are as described herein for heteroarylcycloalkyl, except that the bond to the parent moiety is through an aromatic carbon atom.

“Aralkenyl” means an aryl-alkenyl- group in which the aryl and alkenyl are as previously described. Preferred aralkenyls contain a lower alkenyl group. Non-limiting examples of suitable aralkenyl groups include 2-phenethenyl and 2-naphthylethenyl. The bond to the parent moiety is through the alkenyl.

“Aralkynyl” means an aryl-alkynyl- group in which the aryl and alkynyl are as previously described. Preferred aralkynyls contain a lower alkynyl group. The bond to the parent moiety is through the alkynyl. Non-limiting examples of suitable aralkynyl groups include phenacetylenyl and naphthylacetylenyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, 2-(furan-3-yl)ethyl and quinolin-3-ylmethyl. The bond to the parent moiety is through the alkyl.

“Heteroaralkenyl” means an heteroaryl-alkenyl- group in which the heteroaryl and alkenyl are as previously described. Preferred heteroaralkenyls contain a lower alkenyl group. Non-limiting examples of suitable heteroaralkenyl groups include 2-(pyrid-3-yl)ethenyl and 2-(quinolin-3-yl)ethenyl. The bond to the parent moiety is through the alkenyl.

“Heteroaralkynyl” means an heteroaryl-alkynyl- group in which the heteroaryl and alkynyl are as previously described. Preferred heteroaralkynyls contain a lower alkynyl group. Non-limiting examples of suitable heteroaralkynyl groups include pyrid-3-ylacetylenyl and quinolin-3-ylacetylenyl. The bond to the parent moiety is through the alkynyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)-, alkyl-C(O)-, alkenyl-C(O)-, Alkynyl-C(O)-, cycloalkyl-C(O)-, cycloalkenyl-C(O)-, or cycloalkynyl-C(O)-group in which the various groups are as previously described. The bond to the parent moiety is through the carbonyl. Preferred acyls contain a lower alkyl. Non-limiting examples of suitable acyl groups include formyl, acetyl, propanoyl, 2-methylpropanoyl, butanoyl and cyclohexanoyl.

“Aroyl” means an aryl-C(O)-group in which the aryl group is as previously described. The bond to the parent moiety is through the carbonyl. Non-limiting examples of suitable groups include benzoyl and 1- and 2-naphthoyl.

“Heteroaroyl” means a heteroaryl-C(O)-group in which the heteroaryl group is as previously described. Non-limiting examples of suitable groups include nicotinoyl and pyrrol-2-ylcarbonyl. The bond to the parent moiety is through the carbonyl.

“Alkoxy” means an alkyl-O-group in which the alkyl group is as previously described. Non-limiting examples of suitable alkoxy groups include methoxy, ethoxy, n-propoxy, isopropoxy, n-butoxy and heptoxy. The bond to the parent moiety is through the ether oxygen.

“Aryloxy” means an aryl-O-group in which the aryl group is as previously described. Non-limiting examples of suitable aryloxy groups include phenoxy and naphthoxy. The bond to the parent moiety is through the ether oxygen.

“Aralkyloxy” means an aralkyl-O-group in which the aralkyl groups is as previously described. Non-limiting examples of suitable aralkyloxy groups include benzyloxy and 1- or 2-naphthalenemethoxy. The bond to the parent moiety is through the ether oxygen.

“Alkylamino” means an —NH₂ or —NH₃⁺ group in which one or more of the hydrogen atoms on the nitrogen is replaced by an alkyl group as defined above.

“Arylamino” means an —NH₂ or —NH₃⁺ group in which one or more of the hydrogen atoms on the nitrogen is replaced by an aryl group as defined above.

“Alkylthio” means an alkyl-S-group in which the alkyl group is as previously described. Non-limiting examples of suitable alkylthio groups include methylthio, ethylthio, i-propylthio and heptylthio. The bond to the parent moiety is through the sulfur.

“Arylthio” means an aryl-S-group in which the aryl group is as previously described. Non-limiting examples of suitable arylthio groups include phenylthio and naphthylthio. The bond to the parent moiety is through the sulfur.

“Aralkylthio” means an aralkyl-S-group in which the aralkyl group is as previously described. Non-limiting example of a suitable aralkylthio group is benzylthio. The bond to the parent moiety is through the sulfur.

“Alkoxycarbonyl” means an alkyl-O—CO-group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)-group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)-group. Non-limiting example of a suitable aralkoxycarbonyl group is benzyloxycarbonyl. The bond to the parent moiety is through the carbonyl.

“Alkylsulfonyl” means an alkyl-S(O₂)-group. Preferred groups are those in which the alkyl group is lower alkyl. The bond to the parent moiety is through the sulfonyl.

“Alkylsulfinyl” means an alkyl-S(O)-group. Preferred groups are those in which the alkyl group is lower alkyl. The bond to the parent moiety is through the sulfinyl.

“Arylsulfonyl” means an aryl-S(O₂)-group. The bond to the parent moiety is through the sulfonyl.

“Arylsulfinyl” means an aryl-S(O)-group. The bond to the parent moiety is through the sulfinyl.

The term “cycloalkylene” refers to substitution on the same carbon atom in an alkylene group with a cyclic group. Nonlimiting examples include

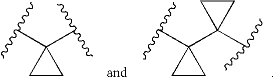

The term “optionally substituted” means optional substitution with the specified groups, radicals or moieties, in available position or positions.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art.

As used herein, the term “composition” is intended to encompass a product comprising the specified ingredients in the specified amounts, as well as any product which results, directly or indirectly, from combination of the specified ingredients in the specified amounts.

The wavy line [symbol] as a bond generally indicates a mixture of, or either of, the possible isomers, e.g., containing (R)- and (S)-stereochemistry. For example,

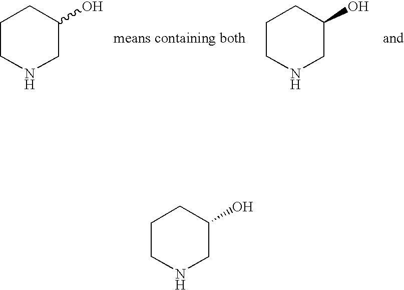

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Effective amount” or “therapeutically effective amount” is meant to describe an amount of compound or a composition of the present invention effective in inhibiting gamma-secretase and thus producing the desired therapeutic effect in a suitable patient.

The compounds of formula I form salts which are also within the scope of this invention. Reference to a compound of formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the formula I may be formed, for example, by reacting a compound of formula I with an amount of acid or base, such as an equivalent amount, in a medium such as one in which the salt precipitates or in an aqueous medium followed by lyophilization.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like. Additionally, acids which are generally considered suitable for the formation of pharmaceutically useful salts from basic pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Compounds of the invention with a carboxylic acid group can form pharmaceutically acceptable esters with an alcohol. Examples of suitable alcohols include methanol and ethanol.

Compounds of formula I, and salts, solvates and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prod rugs of the inventive compounds.

Any formula, compound, moiety or chemical illustration with otherwise unsatisfied valences in the present specification and/or claims herein is assumed to have the requisite hydrogen atom(s) to satisfy the valences.

Those skilled in the art will appreciate that the term “neurodegenerative disease” has its commonly accepted medical meaning and describes diseases and conditions resulting from abnormal function of neurons, including neuronal death and abnormal release of neurotransmitters or neurotoxic substances. In this instance it also includes all diseases resulting from abnormal levels of beta amyloid protein. Examples of such diseases include, but are not limited to, Alzheimer's disease, age-related dementia, cerebral or systemic amyloidosis, hereditary cerebral hemorrhage with amyloidosis, and Down's syndrome.

Lines drawn into the ring systems, such as, for example:

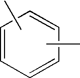

indicate that the indicated line (bond) may be attached to any of the substitutable ring carbon atoms.

As well known in the art, a bond drawn from a particular atom wherein no moiety is depicted at the terminal end of the bond indicates a methyl group bound through that bond to the atom. For example:

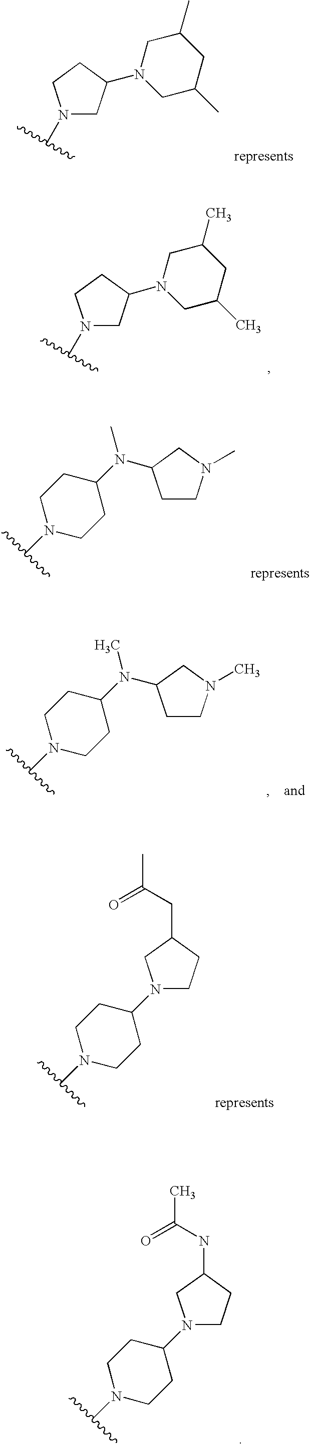

Referring to formula I, examples of Z in the moiety

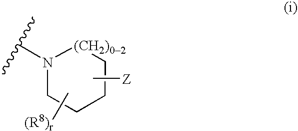

include, but are not limited to:

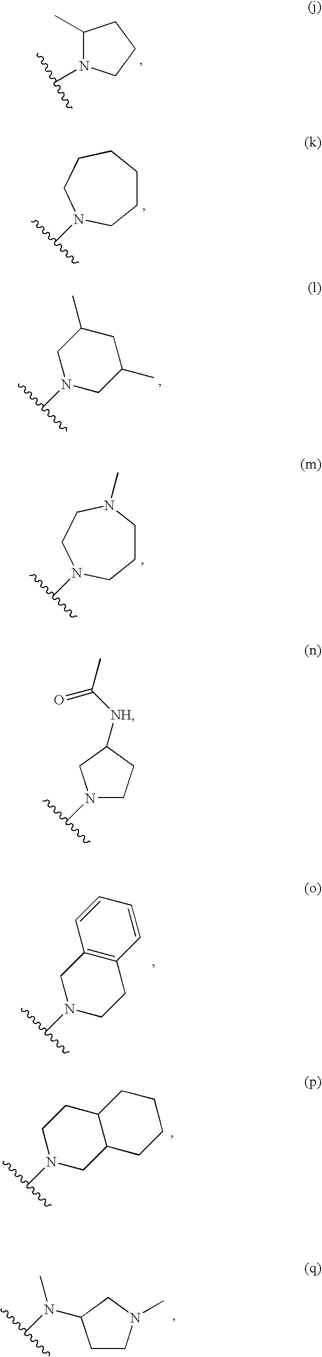

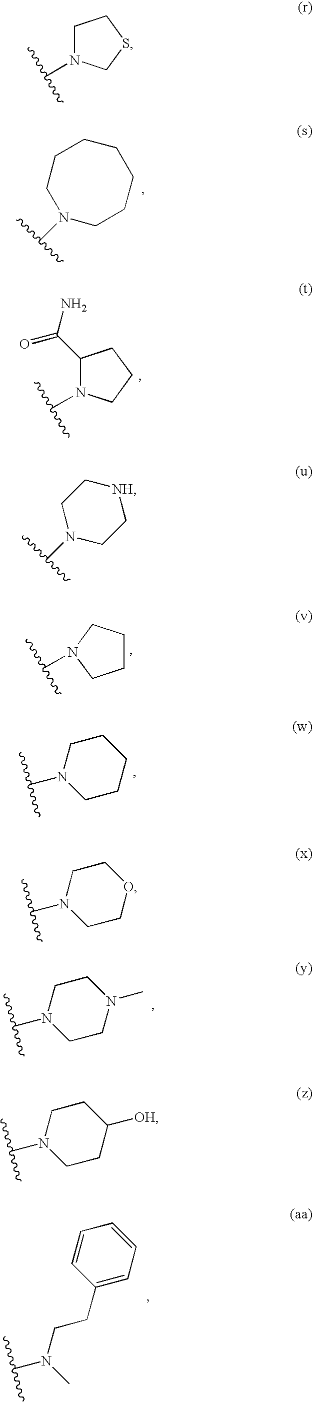

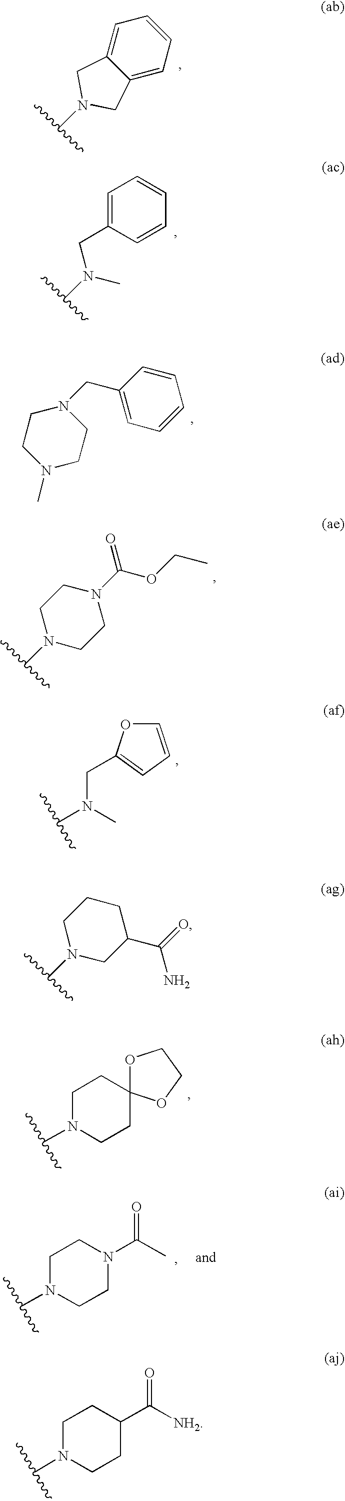

Referring to formula 1, examples of the Y group in —X—C(O)—Y— or —X—CO—Y— include, but are not limited to:

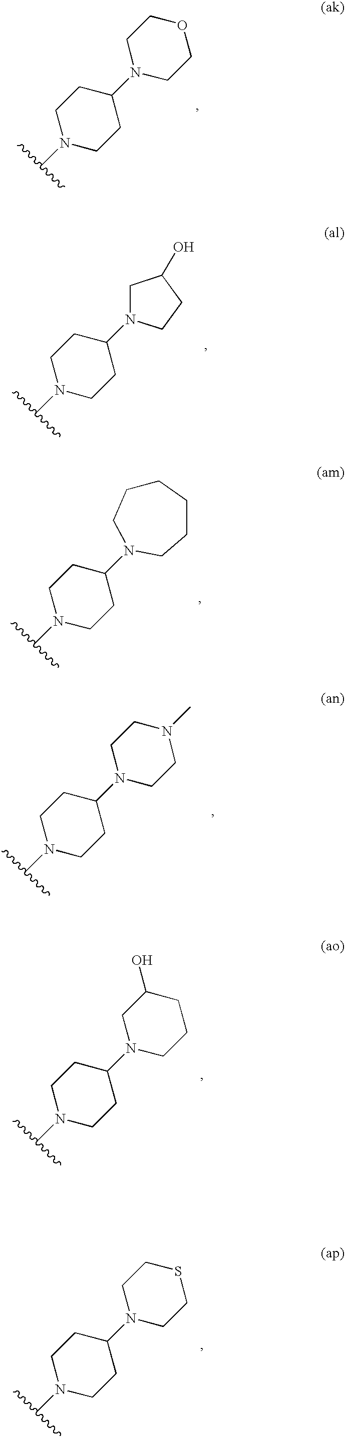

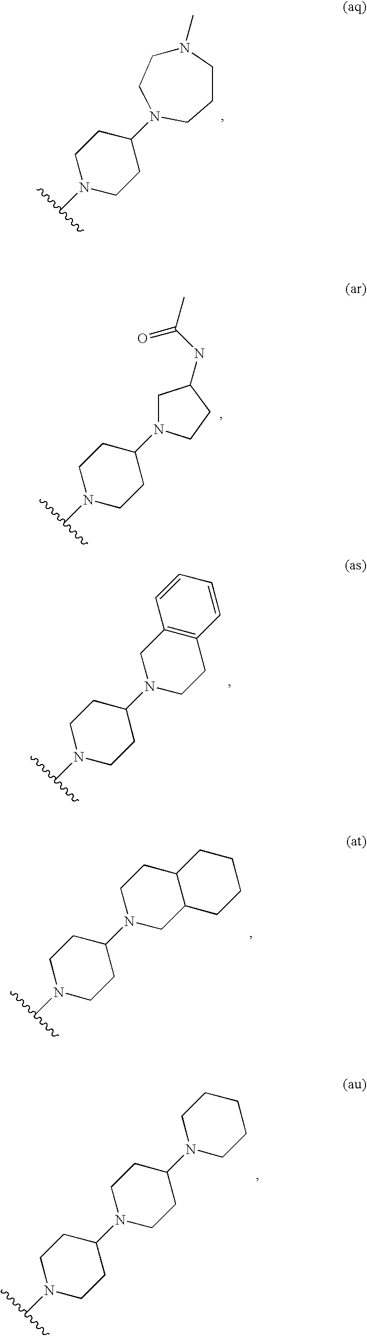

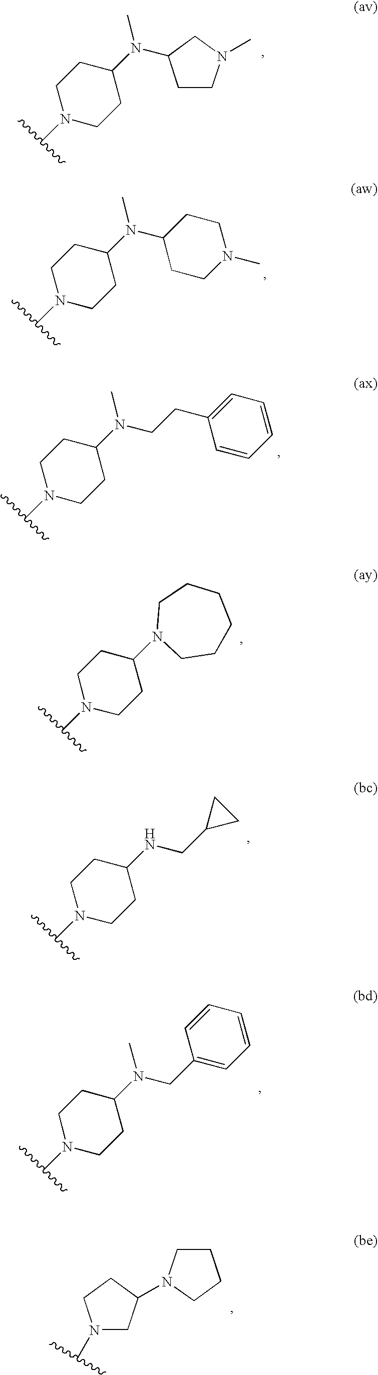

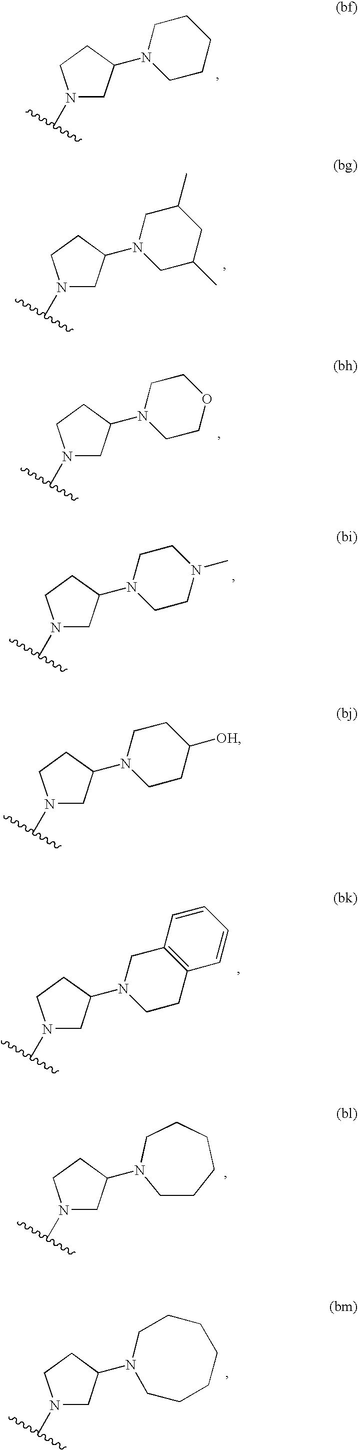

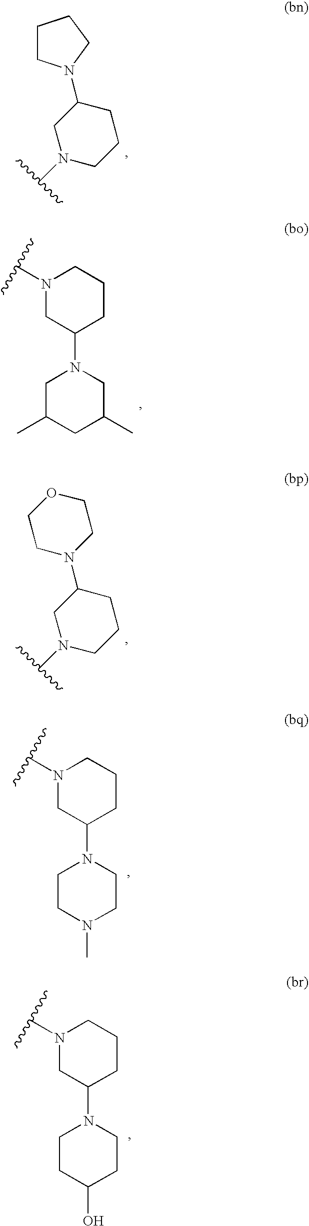

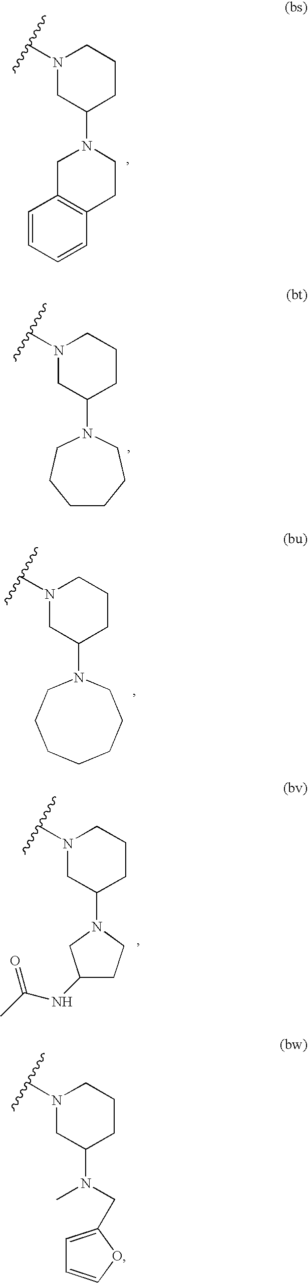

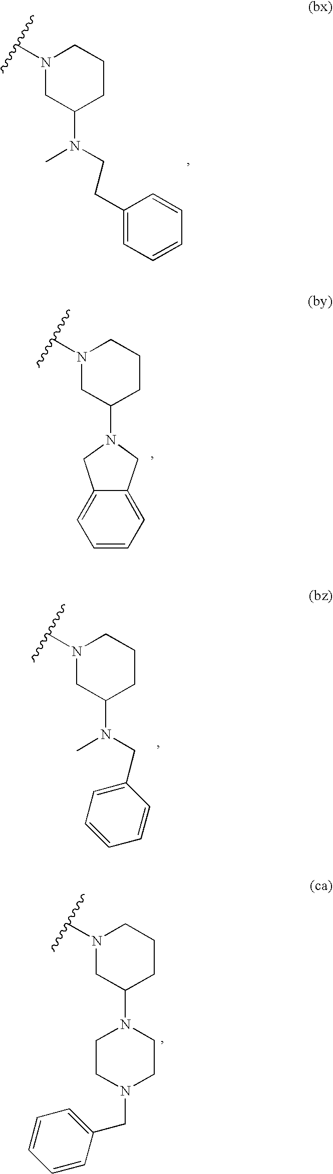

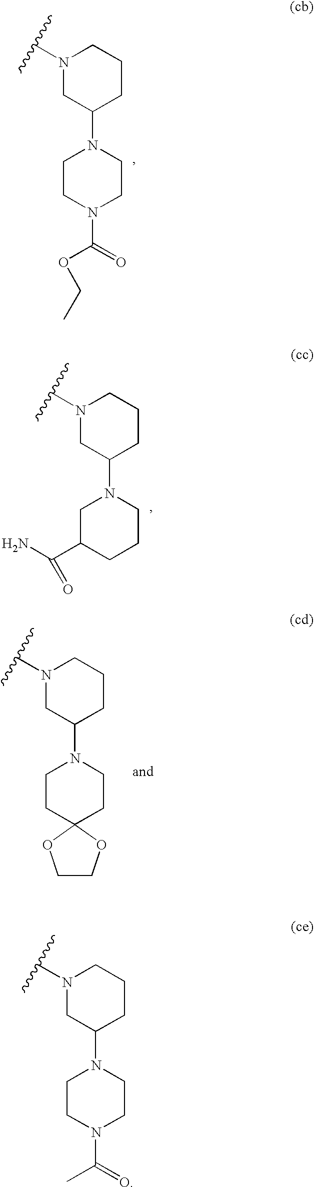

Preferably R¹ is aryl substituted with one or more R⁵ groups, most preferably phenyl substituted with one or more R⁵ groups, and more preferably phenyl substituted with one or more (e.g., 1-3) halo atoms, and still more preferably phenyl substituted with one halo atom, and even still more preferably phenyl substituted with chloro (e.g., p-chlorophenyl).

Preferably n is 0 or 1, o is 0 or 1, and m is 1, 2 or 3, such that m+n+o is 3, and most preferably n and o are independently 0 and m is 3.

Preferably, p is 0 or 1, and most preferably 0.

Preferably, r is 0 or 1, and most preferably 1.

Preferably, s is 0.

Preferably, R² is —XC(O)Y, —(C₁-C₆)alkylene-XC(O)Y, —CH(C₁-C₂alkyl)-X—C(O)—Y (e.g., —CH(CH₃)—X—C(O)—Y), —C(C₁-C₂alkyl)₂-X—C(O)—Y, (spirocyclic-substituted alkyl)-X—C(O)—Y, —CH₂—X—C(O)—NR³—Y, —CH₂—X—C(O)—Y or —CH₂—X—C(O)—NR³—Y, wherein each alkyl is the same or different, —(C₃-C₆)cycloalkylene-XC(O)Y, most preferably —(C₁-₆)alkylene-XC(O)Y or —(C₃-C₆)cycloalkylene-XC(O)Y, more preferably —(C₁-C₆)alkylene-XC(O)Y or —(C₃-C₆)cycloalkylene-XC(O)Y wherein X is —O— or —NH—, still more preferably —(C₁-C₆)alkylene-XC(O)Y or —(C₃-C₆)cycloalkylene-XC(O)Y wherein X is —O—, yet more preferably —CH₂—X—C(O)—Y or

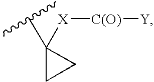

still yet more preferably —CH₂—X—C(O)—Y or

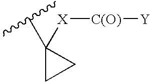

wherein X is —O— or —N(H)—, and even still more preferably —CH₂—X—C(O)—Y or

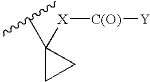

wherein X is —O—.

Preferably, R³ is H.

Preferably, R⁸ is H, —(C₁-C₆)alkyl, or —OH, and most preferably H or methyl.

Preferably, R⁹ is H, —(C₁-C₆)alkyl (e.g., methyl), —(C₁-C₆)alkyl substituted with 1 to 4 —OH groups (e.g., —(CH₂)₂OH), —(C₁-C₆)alkyl-O—(C₁-C₆)alkyl-OH (e.g., 2-(2-hydroxyethoxy)ethyl), (C₃-C₈)cycloalkyl, heteroaryl, or hydroxyalkyl-O-alkyl, and most preferably H, methyl, cyclohexyl, 2-pyridyl, 2-hydroxyethyl or 2-(2-hydroxyethoxy)ethyl;

Preferably, R¹⁰ is H or —(C₁-C₆)alkyl, most preferably H or methyl, more preferably H.

Preferably, R¹¹ is selected from the group consisting of: —(C₁-C₆)alkyl (most preferably methyl or ethyl), (C₃-C₈)-cycloalkyl (most preferably cyclopropyl), aryl (most preferably phenyl), aryl(C₁-C₆)alkyl (most preferably benzyl or —(CH₂)₂phenyl) and —(C₁-C₆)alkoxyalkyl (most preferably —CH₂OCH₃).

Preferably, X is —NH— or —O—, and most preferably —O—.

Preferably Y is —NR⁶R⁷, substituted heterocycloalkyl alkyl, unsubstituted heteroaryl alkyl, unsubstituted aryl alkyl heterocycloalkyl, unsubstituted heterocycloalkyl or unsubstituted cycloalkyl, or Y is selected from the group consisting of:

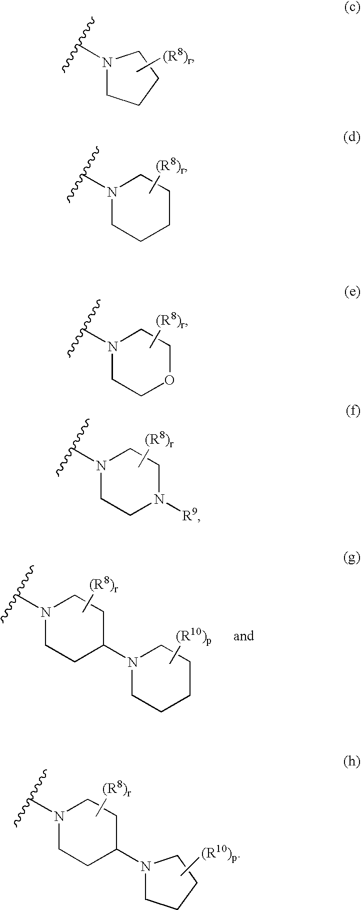

Most preferably, Y is selected from the group consisting of:

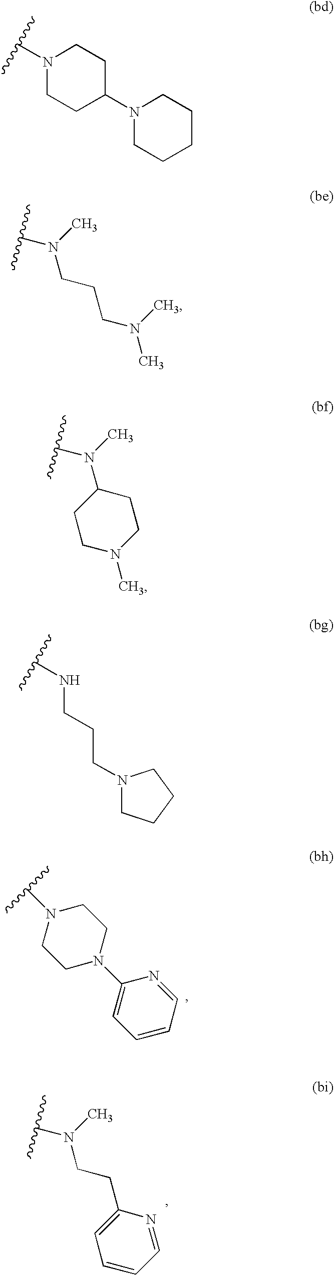

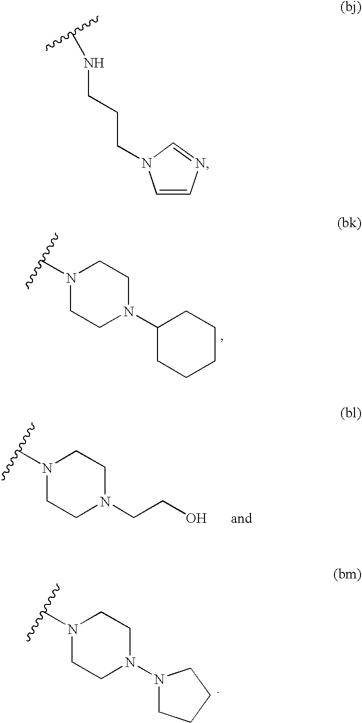

Preferably, R⁶ and R⁷ are independently selected from the group consisting of: H, methyl, ethyl, —(C₃-C₈)cycloalkyl, -aryl(C₁-C₆)alkyl, 4-pyridylmethyl, heterocycloalkyl,

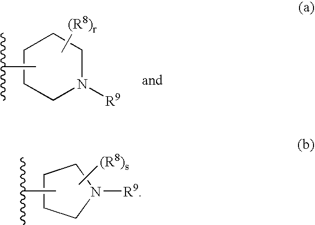

Preferably

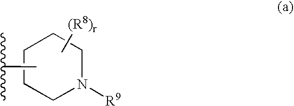

is a group of the formula:

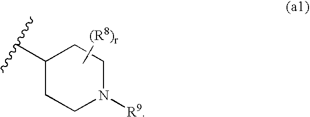

Preferably

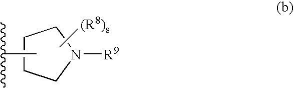

is a group of the formula:

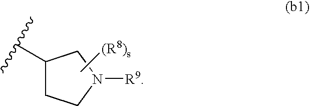

Thus, in one embodiment of the invention:

R¹ is aryl substituted with one or more R⁵ groups, preferably phenyl substituted with one or more R⁵ groups, and most preferably phenyl substituted with one or more halo atoms, and more preferably phenyl substituted with one halo atom, and still more preferably phenyl substituted with chloro (e.g., p-chlorophenyl);

n and o are 0 or 1, and m is 1, 2 or 3, such that m+n+o is 3, and preferably n and o are 0 and m is 3;

p is 0 or 1, and preferably 0;

r is 0 or 1, and preferably 1;

s is 0;

R² is —XC(O)Y, —(C₁-C₆)alkylene-XC(O)Y, —(C₃-C₆)cycloalkylene-XC(O)Y —CH(C₁-C₂alkyl)-X—C(O)—Y (e.g., —CH(CH₃)-X—C(O)—Y), or —C(C₁-C₂alkyl)₂-X—C(O)—Y wherein each alkyl is the same or different, preferably —(C₁-C₆)alkylene-XC(O)Y, or —(C₃-C₆)cycloalkylene-XC(O), most preferably —(C₁ -C₆)alkylene-XC(O)Y or —(C₃-C₆)cycloalkylene-XC(O)Y, wherein X is —O— or —NH—, more preferably —(C₁-C₆)alkylene-XC(O)Y or —(C₃-C₆)cycloalkylene-XC(O)Y, wherein X is —O—, still more preferably —CH₂—X—C(O)—Y or

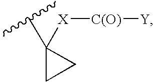

yet still more preferably CH₂—X—C(O)—Y or

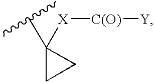

wherein X is —O— or —NH—, and even still more preferably —CH₂—X—C(O)—Y or

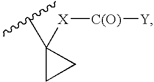

wherein X is —O—;

R³ is H;

R⁸ is H or —(C₁-C₆)alkyl, and preferably H or methyl;

R⁹ is H, —(C₁-C₆)alkyl (e.g., methyl), —(C₁-C₆)alkyl substituted with 1 to 4 —OH groups (e.g., —(CH₂)₂OH), —(C₁-C₆)alkyl-O—(C₁-C₆)alkyl-OH (e.g., 2-(2-hydroxyethoxy)ethyl), (C₃-C₈)cycloalkyl, or heteroaryl, and preferably H, methyl, cyclohexyl, 2-pyridyl, 2-hydroxyethyl or 2-(2-hydroxyethoxy)ethyl;

R₁₀ is H or —(C₁-C₆)alkyl, preferably H or methyl, and most preferably H; and

R¹¹ is selected from the group consisting of: —(C₁-C₆)alkyl (most preferably methyl or ethyl), (C₃-C₈)-cycloalkyl (most preferably cyclopropyl), aryl (most preferably phenyl), aryl(C₁-C₆)alkyl (most preferably benzyl or —(CH₂)₂phenyl) and —(C₁-C₆)alkoxyalkyl (most preferably —CH₂OCH₃); and

the remaining substituents are as defined for formula I.

In another embodiment of the invention:

R¹ is aryl substituted with one or more R⁵ groups, preferably phenyl substituted with one or more R⁵ groups, and most preferably phenyl substituted with one or more halo atoms, and more preferably phenyl substituted with one halo atom, and still more preferably phenyl substituted with chloro (e.g., p-chlorophenyl);

n and o are 0 or 1, and m is 1, 2 or 3, such that m+n+o are 3, and preferably n and o are 0 and m is 3;

p is 0 or 1, and preferably 0;

r is 0 or 1, and preferably 1;

s is 0;

R² is —XC(O)Y, —(C₁-C₆)alkylene-XC(O)Y, —(C₃-C₆)cycloalkylene-XC(O)Y, —CH(C₁-C₂alkyl)-X—C(O)—Y (e.g., —CH(CH₃)—X—C(O)—Y), or —C(C₁-C₂alkyl)₂—X—C(O)—Y wherein each alkyl is the same or different, preferably —(C₁-C₆)alkylene-XC(O)Y or —(C₃-C₆)cycloalkylene-XC(O), and most preferably —CH₂—X—C(O)—Y or

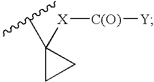

R³ is H;

R⁸ is H or —(C₁-C₆)alkyl, and preferably H or methyl;

R⁹ is H, —(C₁-C₆)alkyl (e.g., methyl), —(C₁-C₆)alkyl substituted with 1 to 4 —OH groups (e.g., —(CH₂)₂OH), —(C₁-C₆)alkyl-O—(C₁-C₆)alkyl-OH (e.g., 2-(2-hydroxyethoxy)ethyl), (C₃-C₈)cycloalkyl, or heteroaryl, and preferably H, methyl, cyclohexyl, 2-pyridyl, 2-hydroxyethyl or 2-(2-hydroxyethoxy)ethyl;

R¹⁰ is H or —(C₁-C₆)alkyl, preferably H or methyl, and most preferably H;

X is —O—;

Y is —NR⁶R⁷; or

Y is selected from the group consisting of:

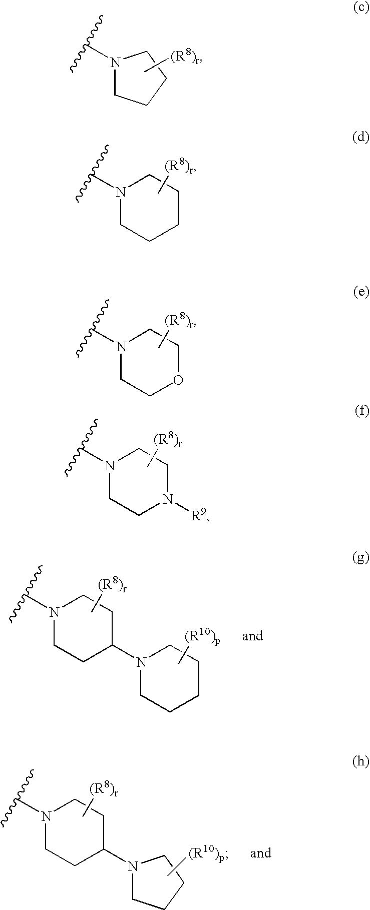

R¹¹ is selected from the group consisting of: —(C₁-C₆)alkyl (most preferably methyl or ethyl), (C₃-C₈)-cycloalkyl (most preferably cyclopropyl) aryl (most preferably phenyl), aryl(C₁-C₆)alkyl (most preferably benzyl or —(CH₂)₂phenyl) and —(C₁-C₆)alkoxyalkyl (most preferably —CH₂OCH₃); and

the remaining substituents are as defined for formula I.

In another embodiment of this invention:

R¹ is aryl substituted with one or more R⁵ groups, preferably phenyl substituted with one or more R⁵ groups, and most preferably phenyl substituted with one or more halo atoms, and more preferably phenyl substituted with one halo atom, and still more preferably phenyl substituted with chloro (e.g., p-chlorophenyl);

n is 0 or 1, o is 0 or 1, and m is 1, 2 or 3, such that m+n+o is 3, and preferably n is 0, o is 0, and m is 3;

p is 0 or 1, and preferably 0;

r is 0 or 1, and preferably 1;

s is 0;

R² is —XC(O)Y, —(C₁-C₆)alkylene-XC(O)Y, —CH(C₁-C₂alkyl)-X—C(O)—Y (e.g., —CH(CH₃)—X—C(O)—Y), or —C(C₁-C₂alkyl)₂-X—C(O)—Y wherein each alkyl is the same or different, preferably —(C₁-C₆)alkylene-XC(O)Y, and most preferably —CH₂—X—C(O)—Y or —(C₃-C₆)cycloalkylene-X—C(O)—Y—;

R³ is H;

R⁸ is H or —(C₁-C₆)alkyl, and preferably H or methyl;

R⁹ is H, —(C₁-C₆)alkyl (e.g., methyl), —(C₁-C₆)alkyl substituted with 1 to 4 —OH groups (e.g., —(CH₂)₂OH), —(C₁-C₆)alkyl-O—(C₁-C₆)alkyl-OH (e.g., 2-(2-hydroxyethoxy)ethyl), (C₃-C₈)cycloalkyl, or heteroaryl, and most preferably H, methyl, cyclohexyl, 2-pyridyl, 2-hydroxyethyl or 2-(2-hydroxyethoxy)ethyl;

R¹⁰ is H or —(C₁-C₆)alkyl, preferably H or methyl, and more preferably H;

X is —O—;

Y is —NR⁶R⁷; or

Y is selected from the group consisting of:

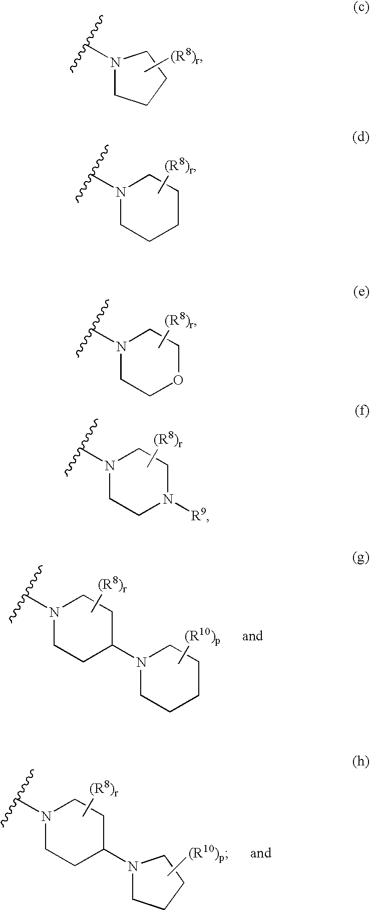

R⁶ and R⁷ are independently selected from the group consisting of: H, methyl, ethyl, —(C₃-C₈)cycloalkyl, -aryl(C₁-C₆)alkyl, 4-pyridylmethyl,

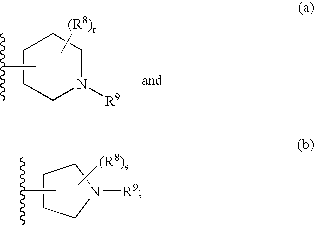

R¹¹ is selected from the group consisting of: —(C₁-C₆)alkyl (preferably methyl or ethyl), (C₃-C₈)-cycloalkyl (preferably cyclopropyl), aryl (preferably phenyl), aryl(C₁-C₆)alkyl (preferably benzyl or —(CH₂)₂phenyl), and —(C₁-C₆)alkoxyalkyl (preferably —CH₂OCH₃); and the remaining substituents are as defined for formula I.

Representative compounds of the invention include but are not limited to the compounds of Examples 1-29, 31-33, 35-48, 50-61, 63-67, 67A-67BS, 68,69, 71-74, 74A, 74B, 74C, 75, 76, 78-83, 85-99,101-159,159A, 159B, 159C, 160, 160A-160AA, 161, 161A-161G, 162, 162A, 162B, 162C, 164, 164A, 164B, 164C, 165-167, 167A, 167B, 167C, 168, 168A, 169, 169A-169D, 170, 170A-170AD, 171-173, 173A-173T, and 174.

Preferred compounds of the invention are the compounds of Examples 7, 61, 67B, 67E, 67N, 67P, 67U, 67AG, 67AT, 67AW, 67AY, 67BA, 67BD, 67BE, 67BG, 67BH, 67BL, 73, 160B, 160K, 161, 161A, 161E, 161 F, 173, 173A, 173B, 173C, 173E, 173G, 173I, 173J, 173K, 173L, 173N. Most preferred compounds are the compounds of Examples 7, 61, 67-B, 67-AT, 67-BG, 73, 161-A, 173, 173-A, 173-C, 173-E, 173-J, 173-N, 173-P, 173-Q, 173-R, 173-S, 173-T AND 173-U.

Compounds of formula I can be prepared by various methods well known to those skilled in the art, and by the methods described below.

Method 1

In Method 1, compounds of formula I having the structure Ia are prepared.

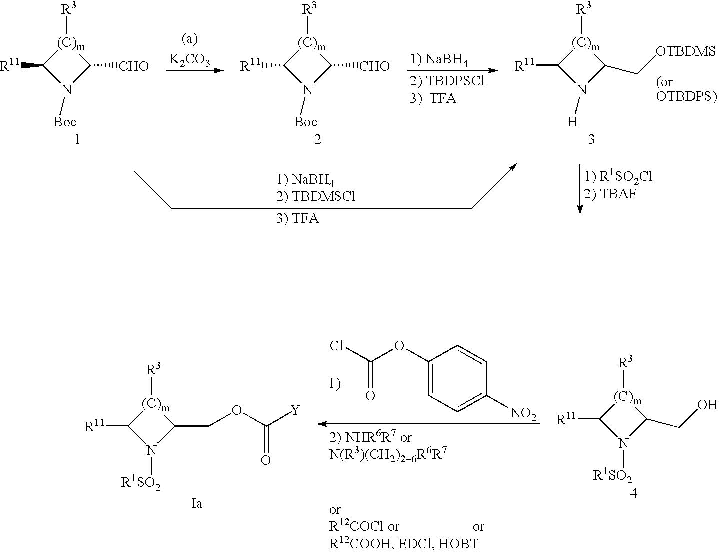

In method 1, R¹² represents the Y substituents defined above in paragraphs (3) to (18) of the definition of Y. When the reagents R¹²COCl or R¹²COOH are used in Method 1, then Y in formula Ia represents R¹². In Method 1, a trans-substituted N-Boc-cyclic amine 2-carboxaldehyde 1 is epimerized to the corresponding cis isomer using a mild base such as potassium carbonate (path a). The cis geometry is retained in all subsequent steps. Alternatively, the epimerization step can be omitted to yield trans products (path b). Aldehyde 2 is reduced using a reducing agent such as sodium borohydride. The alcohol is protected using a typical protecting group such as a t-butyldiphenylsilyl ether, and the boc group is removed under acidic conditions to give 3. The cyclic amine is converted to a sulfonamide by reaction with a sulfonyl halide, and the alcohol protecting group is removed under standard conditions to give 4. Alcohol 4 can be converted to a variety of compounds of type Ia using methods well-known to those skilled in the art. For example, carbamates can be prepared by reaction of 4 with 4-nitrophenylchloroformate followed by reaction of the resulting carbonate with a primary or secondary amine. Alternatively, esters can be prepared by reaction of 4 with either an acid halide of a carboxylic acid in the presence of a suitable coupling reagent such as EDCl and HOBT.

Starting material of formula 1 in Method 1 are known in the art or can be prepared as described below.

Method 2

In Method 2, compounds of formula I having the structure Ib are prepared.

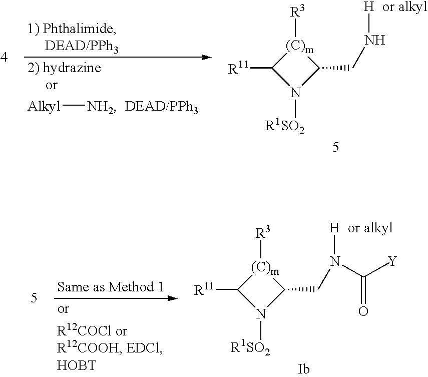

In Method 2, R¹² is as defined in Method 1

In Method 2, alcohol 4 from method 1 converted to the corresponding primary or secondary amine under a variety of conditions, such as by reaction with phthalimide under Mitsunobu conditions followed by treatment with hydrazine or by reaction with a primary amine under Mitsunobu conditions. The resulting amine is converted to ureas or to amides Ib using the same procedures described for carbamates and esters in Method 1.

Methods 3-A and 3-B

In Methods 3-A and 3-B, compounds of formula I having the structure Ic are prepared.

Method 3-A

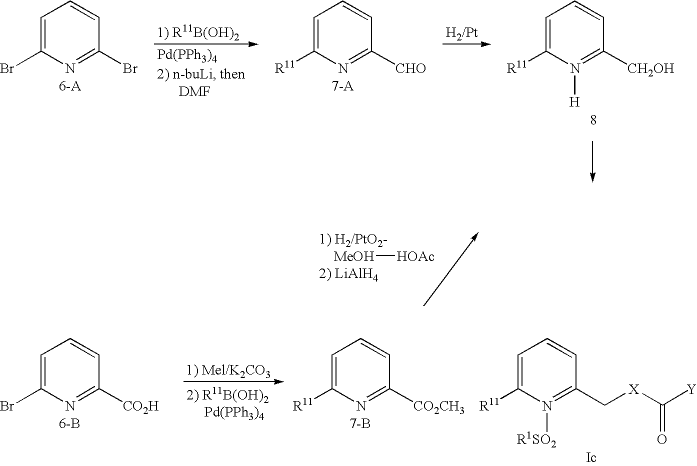

In Method 3-A, 2,6-dibromopyridine is reacted with a boronic acid derivative R¹¹B(OH)₂ (most preferably an aryl or vinyl boronic acid) in the presence of a palladium catalyst. The resulting 6-substituted 2-bromopyridine is formylated by treatment with an alkyl lithium such as n-butyllithium followed by treatment with a formylating agent such as dimethylformamide to give 7-A. This product is hydrogenated to give alcohol 8 (where any unsaturation in R¹¹ may also have been reduced). Alcohol 8 can be converted to compounds of formula Ic using the procedures previously described.

Method 3-B

In Method 3-B, 6-bromopicolinic acid 6-B is converted to its methyl ester under standard conditions followed by reaction with a boronic acid derivative R¹¹B(OH)₂ (most preferably an aryl or vinyl boronic acid) in the presence of a palladium catalyst to give 7-B. This is then hydrogenated using a suitable catalyst such as platinum oxide, preferably in the presence of acetic acid, then reduced with a hydride reagent such as lithium aluminum hydride to give alcohol 8. Alcohol 8 can be converted to compounds of formula Ic using the procedures previously described.

Method 4

In Method 4, compounds of formula I having the structure Id are prepared wherein R¹¹ in 9 and Id represents alkyl having at least two carbons, arylalkyl, or heteroarylalkyl.

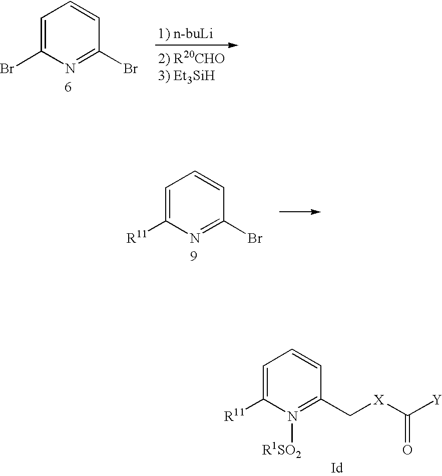

In Method 4, R²⁰ represents alkyl, unsubstituted aryl, substituted aryl, unsubstituted arylalkyl, substituted arylalkyl, unsubstituted heteroaryl, substituted heteroaryl, unsubstituted heteroarylalkyl, or substituted heteroarylalkyl, wherein these groups are as defined for R¹¹ above.

In Method 4, 2,6-dibromopyridine is mono-metallated under a variety of conditions, such as treatment with an alkyllithium at about −78° C. or by treatment with a lithium trialkylmagnesiumate complex at −10 to 0° C. The resulting organometallic derivative is reacted with an aldeyde R²⁰CHO, and the product is deoxygenated under a variety of conditions, such as by treatment with triethylsilane, to give 9. Compound 9 is formylated and the resulting formyl derivative converted compounds of type Id using the procedures previously described.

Method 5

In Method 5, compounds of formula I having the structure Ie are prepared wherein R¹¹ in 10 and Ie represents alkyl having at least three carbons, arylalkyl wherein said alkyl moiety has at least two carbons, or heteroarylalkyl wherein said alkyl moiety has at least two carbons.

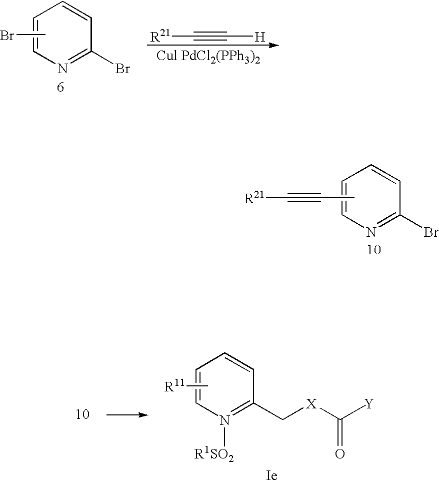

In Method 5, R²¹ represents alkyl, unsubstituted aryl, substituted aryl, unsubstituted arylalkyl, substituted arylalkyl, unsubstituted heteroaryl, substituted heteroaryl, unsubstituted heteroarylalkyl, or substituted heteroarylalkyl, wherein these groups are as defined for R¹¹ above.

In Method 5, 2,6-dibromopyridine is coupled with a mono-substituted alkyne in the presence of a catalyst such as PdCl₂(PPh₃)₄ /CuI. The resulting product is formylated, hydrogenated, and converted to compounds Ie using the procedures previously described.

Method 6

In Method 6, compounds of formula I having the structure Ie are prepared wherein R¹¹ in 12 and If represents alkyl having at least three carbons, arylalkyl wherein said alkyl moiety has at least two carbons, or heteroarylalkyl wherein said alkyl moiety has at least two carbons.

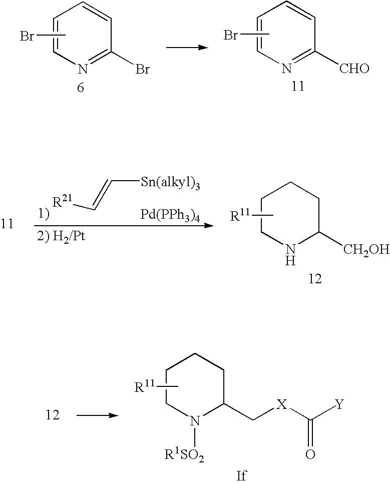

In Method 6, R²¹ represents alkyl, unsubstituted aryl, substituted aryl, unsubstituted arylalkyl, substituted arylalkyl, unsubstituted heteroaryl, substituted heteroaryl, unsubstituted heteroarylalkyl, or substituted heteroarylalkyl, wherein these groups are as defined for R¹¹ above.

In Method 6, 2,6-dibromopyridine is mono-metallated as previously described and the resulting organometallic is reacted with a formylating agent such as DMF to give 11. This compound is reacted with a vinyl tin reagent in the presence of a catalyst such as Pd(PPh₃)₄, and the resulting product is hydrogenated to give 12. Compound 12 is converted to compounds If as previously described.

Method 7

In Method 7, compounds of formula I having the structure Ig are prepared.

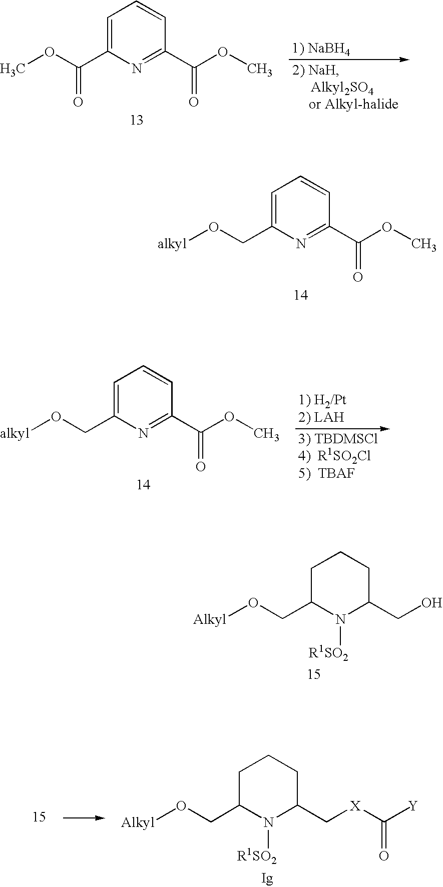

In Method 7, pyridine-2,6-dicarboxylic acid dimethyl ester is reacted with a reducing agent such as sodium borohydride, and the resulting monohydroxymethyl derivative is treated with an alkylating agent such as an alkyl halide or alkylsulfonate to give 14. This is hydrogenated over a catalyst such as platinum oxide, and then reacted with a reducing agent such as lithium aluminum hydride to provide an intermediate cyclic amino alcohol. The alcohol function is protected using a typical protecting group such as a t-butyldimethylsilyl ether, the cyclic amine is converted to a sulfonamide by reaction with a sulfonyl halide, and the alcohol protecting group is removed under standard conditions to give 15. Compound 15 is converted to compounds of type Ig using the methods previously described.

Method 8

In Method 8, compounds of formula I having the structure Ih are prepared.

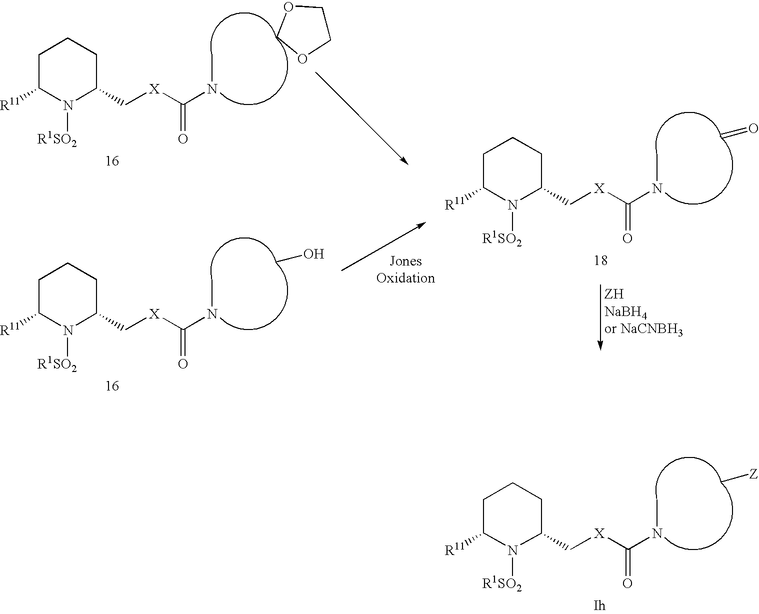

In Method 8, ketal 16 or alcohol 17 are prepared using the procedures described in Method 1 and Method 2. These are converted to the corresponding ketone by either acid hydrolysis of 16 or by oxidation of 17. The ketone is converted to compounds of type Ih by reaction with a primary or secondary amine in the presence of a reducing agent such as sodium borohyride, sodium cyanoborohydride, sodium triacetoxyborohydride, or polymer-bound derivatives thereof.

Method 9

In Method 9, compounds of formula I having the structure Ii and Ij are prepared.

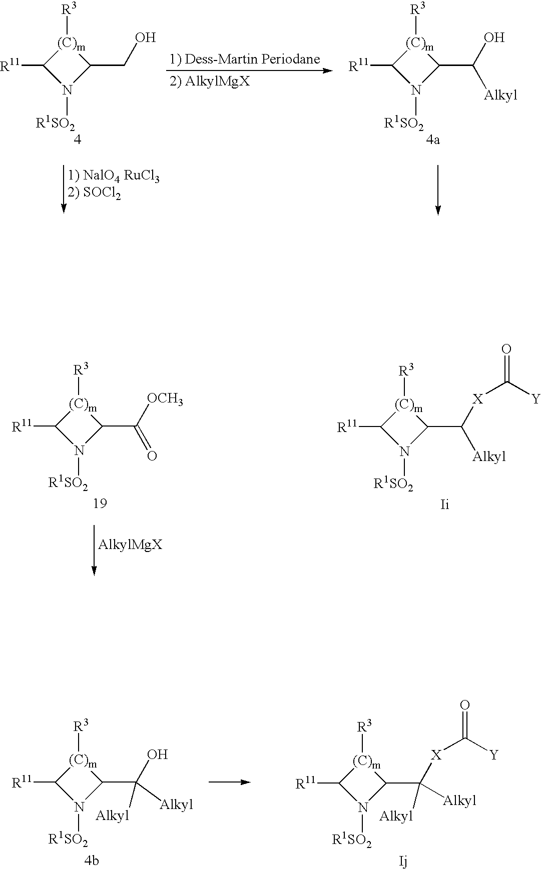

In Method 9, intermediate 4 prepared via any of the methods previously described can be oxidized to an aldehyde using a variety of well-known reagents such as Dess-Martin Periodane. The aldehyde is then treated with an alkylmetal reagent such as a Grignard reagent, an alkyllithium reagent, or an alkylzinc reagent to give alcohol 4a. Intermediate 4a can be converted to compounds of type Ii using the procedures described in Methods 1 through 8. Alternatively, 4 can be converted to ester 19 and then treated with a Grignard reagent to give 4b. This is converted to compounds of type Ij as previously described.

Compounds of type 1k are prepared according to Method 10.

Method 10:

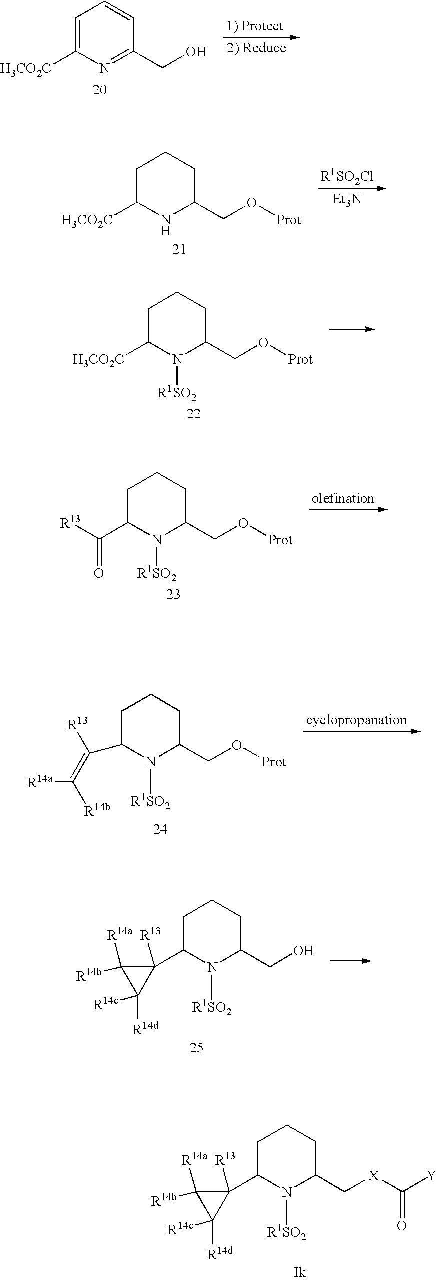

Ester 20 is protected with a suitable protecting group (Prot) such as t-butyldimethylsilyl ether, and the pyridine is reduced by well-known methods such as by treatment with hydrogen gas in the presence of a catalyst such as platinum oxide in a solvent such as ethanol or ether, to give piperidine 21. This is sulfonylated by treatment with a sulfonyl halide in the presence of a base such as triethylamine to give 22. Using well-known methods, the ester of 22 can be converted to 23, where R¹³ is H or alkyl. For instance, 22 can be reduced to the corresponding aldehyde (23, R¹³═H) by treatment with DIBAL. The aldehyde can be treated with a Grignard reagent followed by oxidation to give a ketone (23, R¹³≠H). Compound 23 can be converted to olefin 24 using well-known methods such as by treatment with a alkyl phosphonium ylide. Olefin 24 can be converted to cyclopropane 25 by well-known methods, for instance, by treatment with a dihalomethane such as diiodomethane in the presence of dialkylzinc and optionally in the presence of trifluoroacetic acid, by treatment with an alkyl or substituted alkyldiazo compound in the presence of a metal such as rhodium chloride, or by treatment of an alkyl halide or substituted alkyl halide with a base such as potassium hydroxide. In the above example, R^{14a}, R^{14b}, and R^{14c}═H, alkyl, aryl, halo, —OH, —O(alkyl), —NH₂, —N(H)alkyl, N(alkyl)₂, or C(O)Oalkyl. Compound 25 can be converted to compounds of type Ik using the methods previously described.

Compounds of type 1l are prepared as described in method 11.

Method 11:

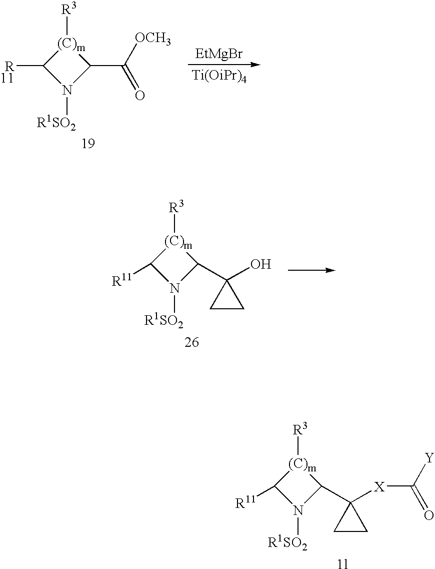

Intermediate 19 of method 9 is treated with ethylmagnesium bromide in the presence of Ti(OiPr)₄ to give cyclopropanol 26, which is converted to compounds of type 1l as previously described.

Compounds of type 1m, wherein R¹¹ is a heteroaryl moiety can be made by several methods as shown below.

Method 12:

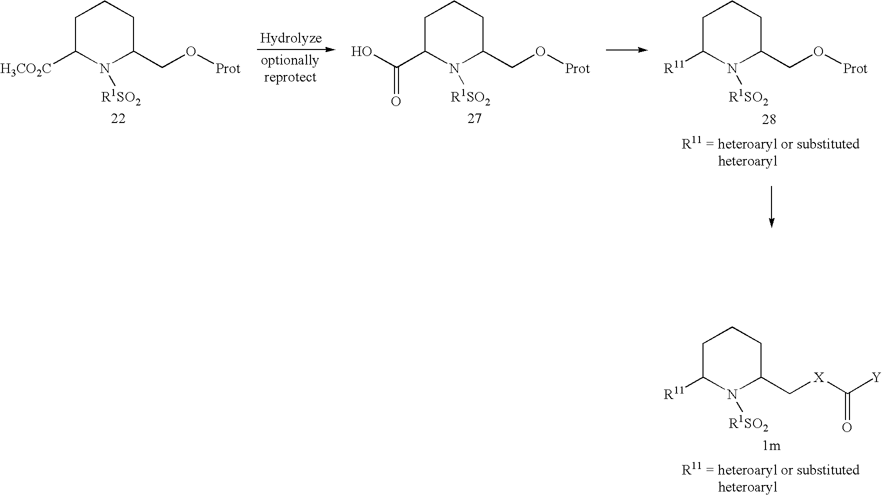

Intermediate 22 from method 10 can be hydrolyzed and, optionally as needed, reprotected to give acid 27. This acid can be transformed to a variety of heteroaryl moieties using methods well-known to those skilled in the art. For instance, coupling with 2-aminoethanol followed by oxidation and dehydrative cyclization according to the method of Morwick et al (Organic Letters 2002, 2665) gives 28 where R¹¹=2-oxazolyl. Compounds of type 28 can be transformed into compounds of type 1m using the methods described earlier.

Method 13:

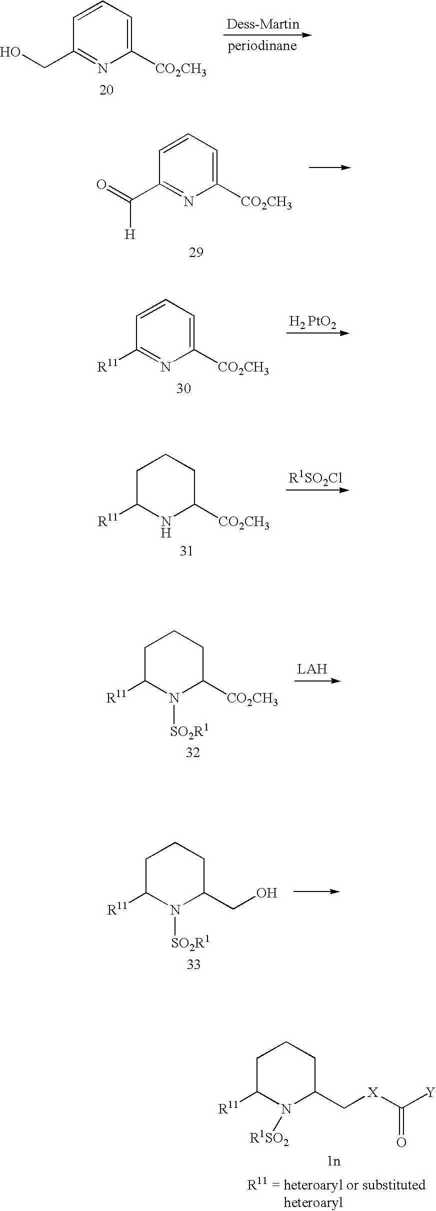

Intermediate 20 from method 10 can be oxidized to aldehyde 29 using, for instance, Dess-Martin periodinane. Aldeyde 29 can be transformed into a variety of intermediates 30 where R¹¹ is heteroaryl using well-known methods. For instance, treatment of 29 with glyoxal and ammonia gives 30 where R¹¹ is 2-imidazolyl. Intermediate 30 can be reduced to piperidine 31 and sulfonylated to give 32 as previously described, and the ester of 32 can be reduced to alcohol 33 using, for instance, lithium aluminum hydride. Intermediate 33 can be transformed to compounds 1n as previously described.

Compounds of this invention of type 1o can be prepared according to method 14:

Method 14:

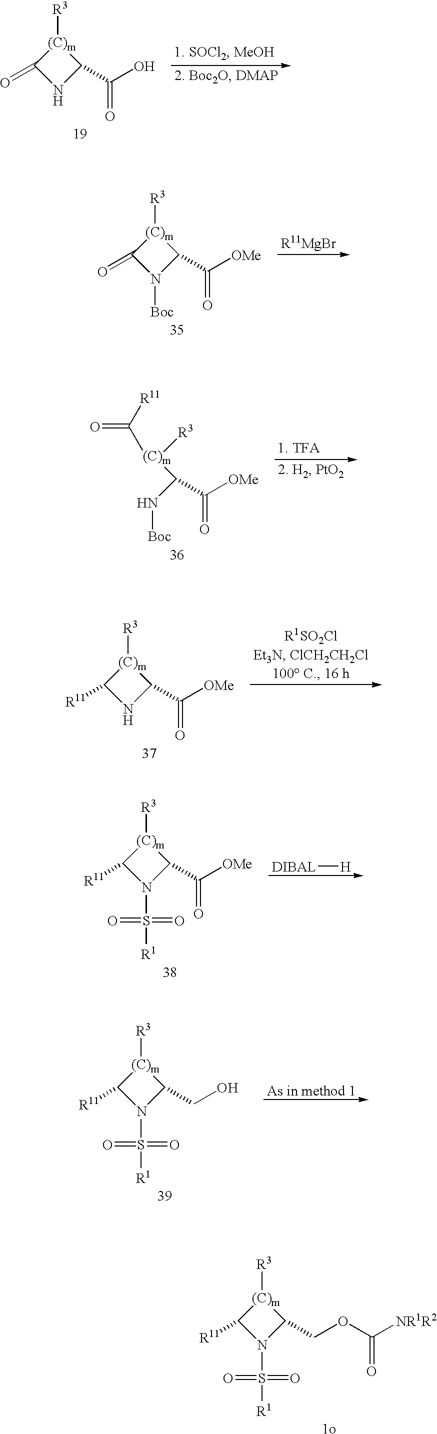

Carboxylactam 34, where m and R³ are as previously defined, is converted to boc-protected ester 35 by standard procedures. This is reacted with an organometallic reagent such as a grignard reagent or organolithium to give ketone 36. The boc group is removed by treatment with an acid such as trifluoroacetic acid and the resulting compound undergoes reductive cyclization in the presence of a suitable reducing agent such as by treatment with hydrogren and a catalyst such as PtO2, to give 37. This is converted to the corresponding sulfonamide by treatment with a sulfonyl halide in the presence of a base such as triethylamine. The ester is reduced to give alcohol 39, which is converted to compounds of type 1o by the methods previously described.

Chiral compounds of this invention can be resolved by chromatography over a chiral stationary phase as described in the examples.

### EXAMPLES

The invention disclosed herein is exemplified by the following examples which should not be construed as limiting the scope of the invention. Alternative mechanistic pathways and analogous structures within the scope of the invention may be apparent to those skilled in the art.

Abbreviations Used:

AcOEt represents: ethyl acetate;

AcOH represents: acetic acid;

DCM represents: dichloromethane;

DEAD represents: diethylazodicarboxylate;

DMAP represents 4-dimethylaminopyridine;

DMF: represents dimethylformamide;

EDCl represents: 1-(3-dimethylaminopropyl)-3-ethylcarbodiimide;

Et₂O represents: diethyl ether;

EtOAc represents: ethyl acetate;

MeOH represents: methanol;

OTBDMS represents: t-butyldimethylsilyloxy (or t-butyldimethylsilyl ether);

OTBDPS represents: t-butyldiphenylsilyloxy (or t-butyldiphenylsilyl ether);

Ph represents: phenyl;

HOBT represents: 1-hydroxybenzotriazole;

TBAF represents: tetrabutylammonium fluoride;

TBDMSCl represents: t-butyldimethylsilyl chloride

TBDPSCl: represents t-butyldiphenylsilylchloride;

TFA: represents trifluroacetic acid;

THF: represents tetrahydrofuran;

TMS represents: trimethylsilane.

Where NMR data are presented, ¹H spectra were obtained on either a Varian VXR-200 (200 MHz, ¹H), Varian Gemini-300 (300 MHz) or XL-400 (400 MHz) and are reported as ppm down field from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 min—10% CH₃CN, 5 min—95% CH₃CN, 7 min—95% CH₃CN, 7.5 min—10% CH₃CN, 9 min—stop. The retention time and observed parent ion are given.

### Example 1

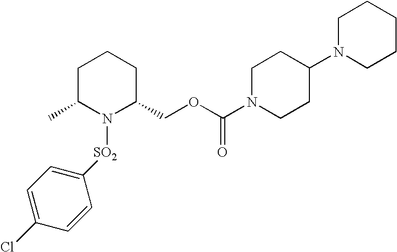

Step 1

Racemic trans 1-(tert-butoxycarbonyl)-2-formyl-6-methyl-piperidine was obtained as described in S. Chackalamannil, R. J. Davies, Y. Wang, T. Asberom, D. Doller, J. Wong, D. Leone and A. T. McPhail, J. Org. Chem. 1999, 64, 1932-1940. A solution of 5.44 g of this aldehyde was stirred in 100 mL of methanol with 6.0 g of K₂CO₃ overnight. Solids were filtered out, and the residue was concentrated. The mixture was redissolved in DCM, washed with water, dried over Na₂SO₄, concentrated and purified chromatographically using 7% ethyl acetate in hexanes as solvent to furnish 3.2 g of product.

Step 2

a) To a solution of 3.21 g (14.1 mmol) of the product of Step 1 in 20.0 mL of THF at 0° C. was added 534 mg (14.1 mmol) of sodium borohydride. The mixture was stirred for 1.5 h, quenched with saturated NaHCO₃, extracted with ether, dried over Na₂SO₄ and freed from solvent in vacuo to give 3.08 g of crude alcohol.

b) The crude alcohol from step 2 was dissolved in 20.0 mL of DMF and treated with 1.83 g (27 mmol) of imidazole and 4.79 g (17.5 mmol) of TBDPSCl. The mixture was stirred overnight, diluted with DCM, washed with water, dried over Na₂SO₄, and solvent was evaporated. The product was purified by chromatography to furnish 4.67 g of TBDPS ether.

c) A solution of 4.67 g of TBDPS ether in 15 mL of DCM was cooled to 0° C. and treated with a mixture containing 30 mL of 99% TFA and 70 mL of DCM. Cooling was removed and the mixture was stirred for 1.5 h. Volatiles were evaporated, the residue was re-evaporated with DCM, re-dissolved in DCM, washed with saturated NaHCO₃, dried over Na₂SO₄, concentrated and passed through a silica gel plug using 5% MeOH in DCM as solvent to yield 3.50 g of product.

Step 3

a) A mixture of 3.50 g (9.53 mmol) of the product of Step 2, 3.02 g of 4-chlorobenzenesulfonyl chloride and 1.92 g (19.06 mmol) of triethylamine in 20.0 mL of DCM was stirred over a period of 48 h. The reaction was washed with saturated NaHCO₃, dried over Na₂SO₄, concentrated and purified by chromatography using 10% ethyl acetate in hexanes as solvent to obtain 4.66 g of sulfonamide.

b) The resulting sulfonamide (4.66 g, 8.61 mmol) was dissolved in 50.0 mL of THF and treated with 17.2 mL (17.2 mmol) of 1M TBAF/THF solution. The mixture was stirred over 1.5 h, poured into water, extracted with ethyl acetate and DCM. The combined organic phases were dried over Na₂SO₄, concentrated and purified by chromatography using gradient of 10-30% ethyl acetate in hexanes as solvent to furnish 2.39 g of product.

Step 4

a) To a mixture of 712 mg (2.3 mmol) of the product of step 3, and 370 mg (4.6 mmol) of pyridine in 10 mL of DCM at 0° C. was added a solution of 4-nitrophenylchlorocarbonate in 5 mL of DCM. The mixture was stirred overnight at ambient temperature, treated with an additional 0.17 mL of pyridine and 100 mg of 4-nitrophenylchlorocarbonate and stirred for additional 5 h. The mixture was diluted with DCM, washed with water, dried over Na₂SO₄, purified by chromatography using 20% ethyl acetate in hexanes as solvent to furnish 860 mg of 4-nitrophenylcarbonate.

b) To a solution of 20 mg of the above product in 0.5 mL of DMF was added 20 mg of 4-(1-piperidino)piperidine. The mixture was allowed to stand overnight, diluted with DCM, washed with 1M NaOH, dried over Na₂SO₄ and purified by prep. TLC (5% MeOH/DCM) to furnish 17 mg of the desired product. 1H NMR (CDCl₃, 300 MHz) δ 7.75 (2H, d, J=8.5 Hz), 7.45 (2H, d, J=8.5 Hz), 4.33-4.20 (4H, m), 4.11-4.00 (2H, m), 2.74 (2H, wide), 2.48-2.34 (5H, ser.m.), 1.80-1.22 (16H, ser.m.), 1.30 (3H, d, J=7.1 Hz); MS (ES) m/e 498.1 (M+H)⁺.

Following procedures similar to those in Example 1, the compounds in Table 1 were prepared.

TABLE 1

| EX. | | |
|---|---|---|
| No. | COMPOUND | Mass Spec |
| 2 | 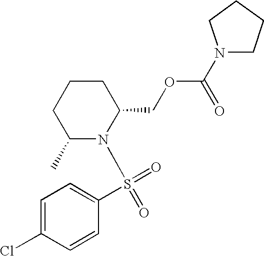 | 401.1 |
| 3 | 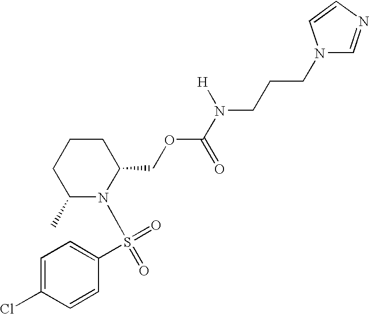 | 455.1 |
| 4 | 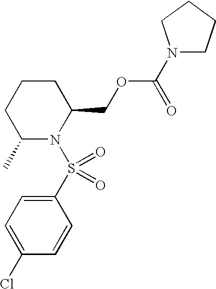 | 401.1 |
| 5 | 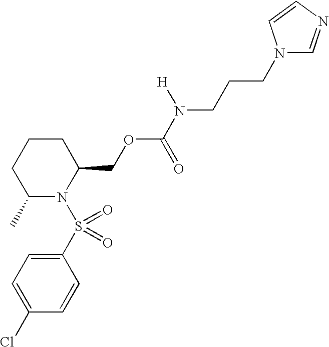 | 455.1 |
| 6 | 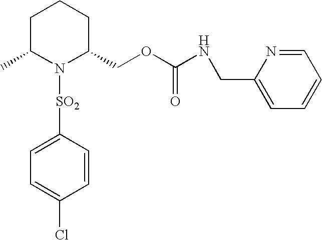 | 438.1 |
| 7 | 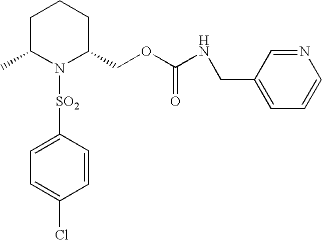 | 438.1 |
| 8 | 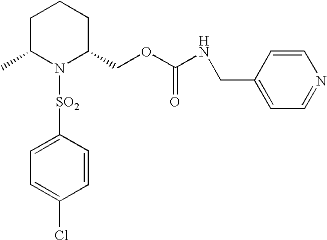 | 438.1 |
| 9 | 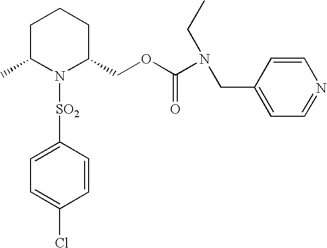 | 466.1 |
| 10 | 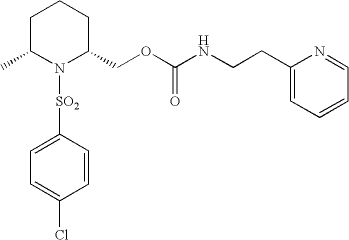 | 452.1 |
| 11 | 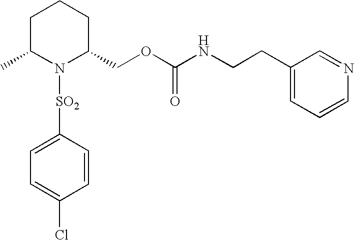 | 452.1 |
| 12 | 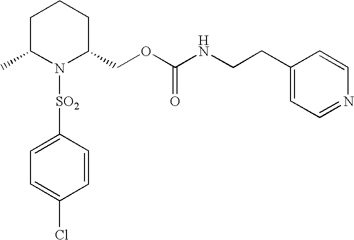 | 452.1 |
| 13 | 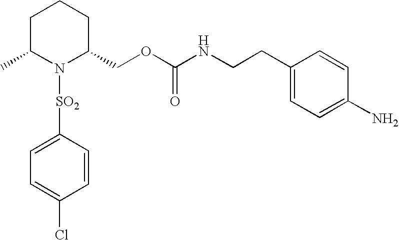 | 466.1 |
| 14 | 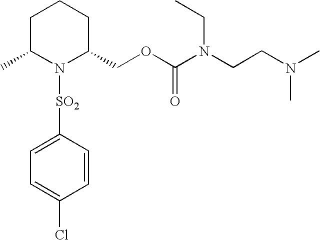 | 446.1 |
| 15 | 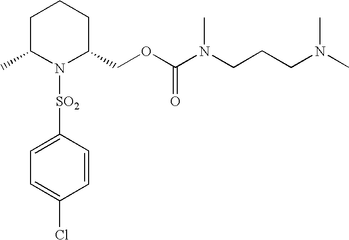 | 446.1 |
| 16 | 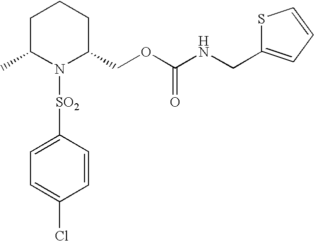 | 443.1 |
| 17 | 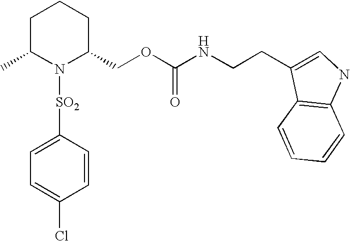 | 490.1 |
| 18 | 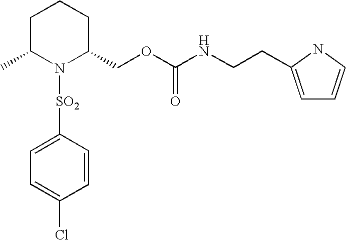 | 441.1 |
| 19 | 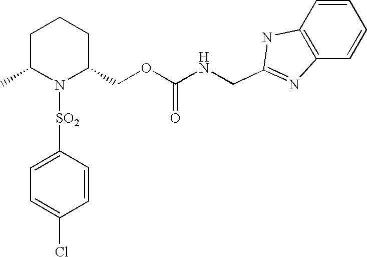 | 477.1 |
| 20 | 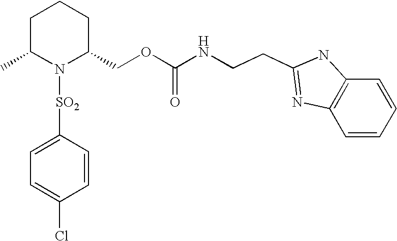 | 491.1 |
| 21 | 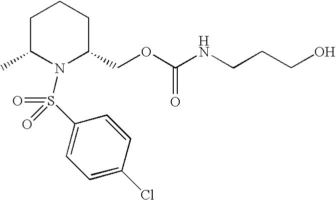 | 405.1 |
| 22 | 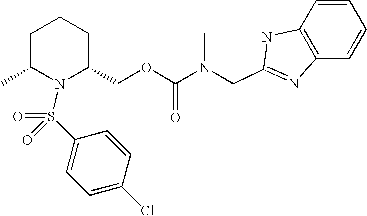 | 491.1 |
| 23 | 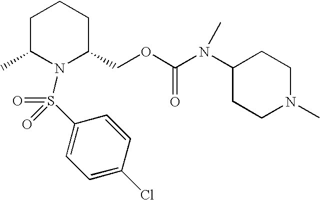 | 458.1 |
| 24 | 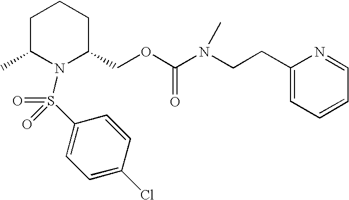 | 466.1 |
| 25 | 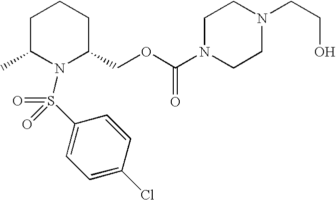 | 460.1 |
| 26 | 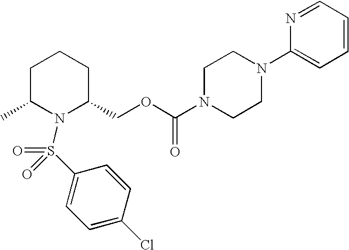 | 493.1 |
| 27 | 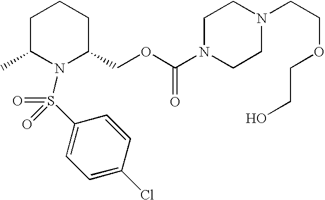 | 504.1 |
| 28 | 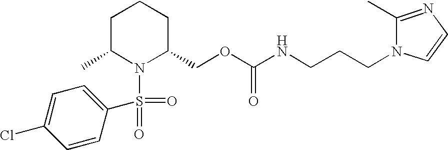 | 469.1 |
| 29 | 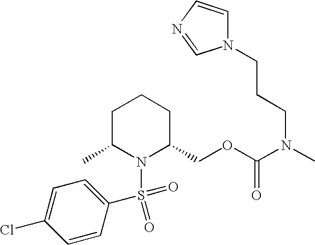 | 469.1 |

### Example 31

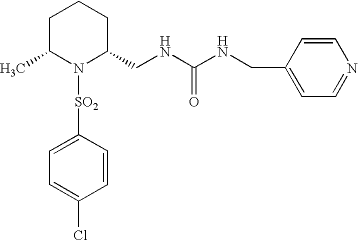

Step 1

a) To a mixture of the product of Example 1, step 3 (425 mg, 1.40 mmol), 308 mg (2.09 mmol) of phthalimide, and 917 mg (3.49 mmol) of triphenylphosphine, was added with stirring 609 mg (3.49 mmol) of DEAD. The mixture was stirred overnight, concentrated in vacuo and purified by column chromatography using 20% ethyl acetate in hexanes. The resulting material was dissolved in 15.0 ml of 1:1 mixture of methanol and DCM and treated with 2 mL of hydrazine. The mixture was stirred over 48 h, partitioned between 1M NaOH solution and DCM, organic phase was washed with 1M NaOH solution to furnish 475 mg of amine.

Step 2

The product of step 1 was transformed to the desired product as described in Example 1, Step 4, using 4-aminomethylpyridine as the amine. 1H NMR (CDCl3 300 MHz) δ 8.56 (2H, d, J=5.5 Hz), 7.71 (2H, d, J=8.2 Hz), 7.48 (2H, d, J=8.2 Hz), 7.29 (2H, d, J=5.5 Hz), 5.14 (2H, m), 4.45 (2H, d, J=6.0 Hz), 4.13 (1H, m), 3.97 (1H, m), 3.53 (1H, m), 3.33 (1H, m), 1.85-1.19 (6H, ser.m.), 1.33 (3H, d, J=7.1 Hz); MS (ES) m/e 437.1 (M+H)⁺.

Following procedures similar to those in Example 31, the compounds in Table 2 were prepared.

TABLE 2

| EX. | | |
|---|---|---|
| No. | COMPOUND | Mass Spec |
| 32 | 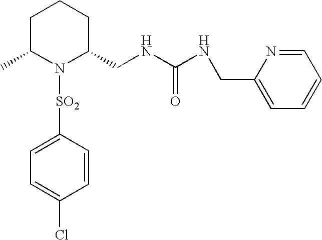 | 437.1 |
| 33 | 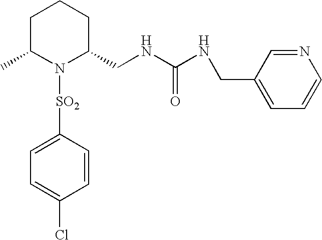 | 437.1 |
| 35 | 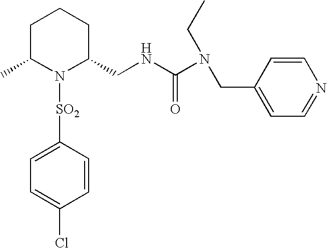 | 465.1 |
| 36 | 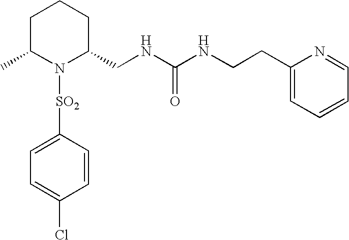 | 451.1 |
| 37 | 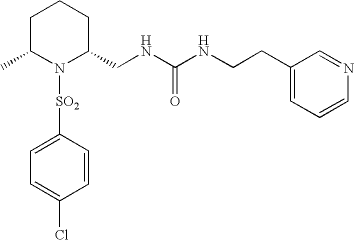 | 451.1 |
| 38 | 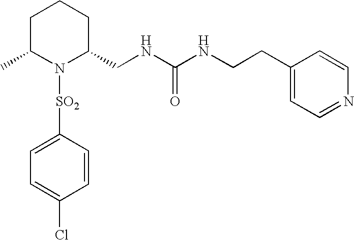 | 451.1 |
| 39 | 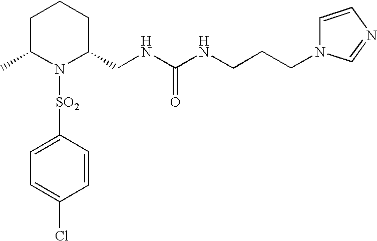 | 454.1 |
| 40 | 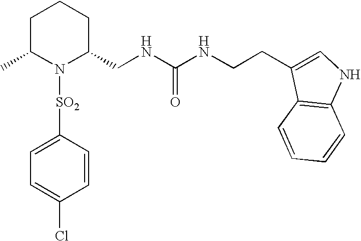 | 489.1 |
| 41 | 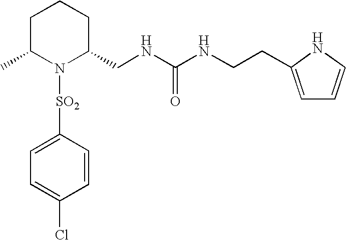 | 440.1 |
| 42 | 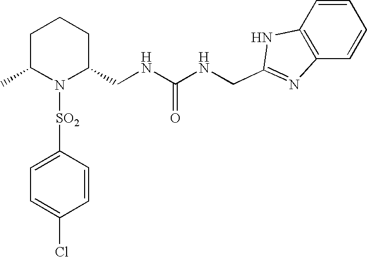 | 476.1 |
| 43 | 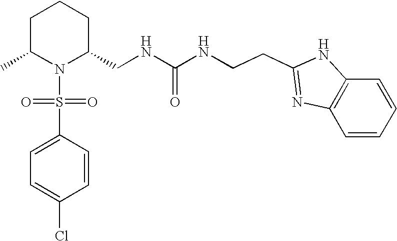 | 490.1 |

### Example 44

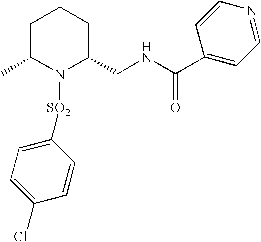

The product of Example 31 step 1 was converted to the title compound by reaction with isonicotinic acid using EDCl and HOBT as coupling reagents, according to the method known in the art. 1H NMR (CDCl3 300 MHz) δ 8.75 (2H, d, J=5.8 Hz), 7.78-7.74 (4H, m), 7.50 (2H, d, J=8.7 Hz), 4.27-4.13 (2H, ser.m), 3.89 (1H, m), 3.39 (1H, dt, J=13.0, 4.3 Hz), 1.81-1.22 (7H, ser.m), 1.35 (3H, d, J=7.3 Hz), MS (ES) m/e 408.1 (M+H)⁺.

Following procedures similar to those in Example 44, the compounds in Table 3 were prepared.

TABLE 3

| EX. | | |
|---|---|---|
| No. | COMPOUND | Mass Spec |
| 45 | 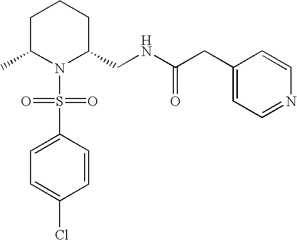 | 422.1 |
| 46 | 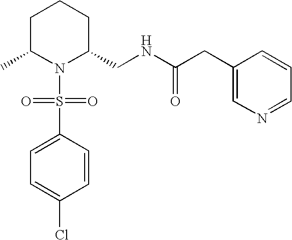 | 422.1 |
| 47 | 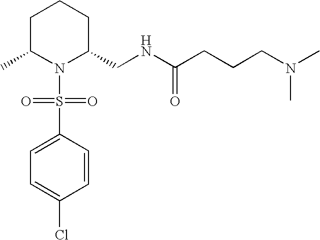 | 416.1 |
| 48 | 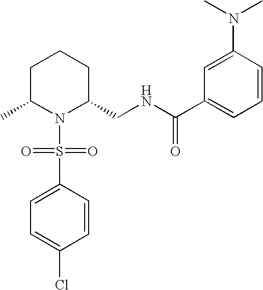 | 450.1 |
| 50 | 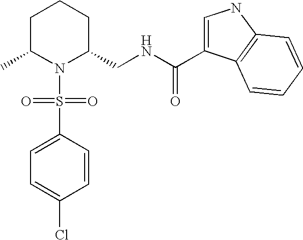 | 446.1 |
| 51 | 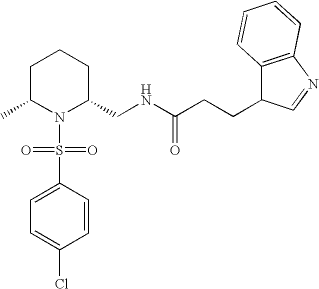 | 474.1 |
| 52 | 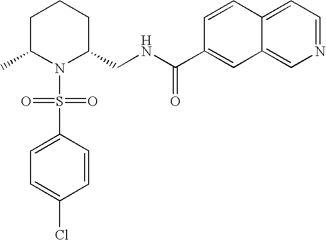 | 458.1 |

### Example 53

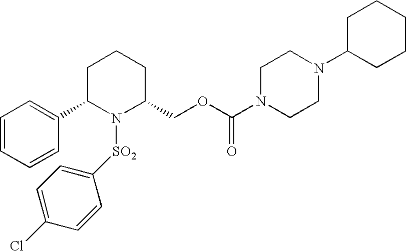

Preparation A: cis (6-Phenyl-piperidin-2-yl)-methanol:

Step 1

(a) To a mixture of 600 mg (2.5 mmol) of 2,6-dibromopyridine in 15 mL of toluene was added a mixture of 150 mg (1.27 mmol) of phenylboronic acid in 5 mL of methanol, 86 mg (0.075 mmol) of Pd(PPh₃)₄ and 15 mL of 2 m Na₂CO₃. The mixture was refluxed overnight, cooled, extracted with ethyl acetate, dried and 2-bromo-6-phenylpyridine isolated chromatographically from unreacted 2,6-dibromopyridine and 2,6-diphenylpyridine.

(b) To a solution of 7.2 g (31.03 mmol) of 2-bromo-6-phenylpyridine in 50 mL of THF at −78° C. was added drop-wise 13.5 mL (31 mmol) of 2.3 M n-BuLi in hexanes followed by 10 mL of DMF. The mixture was stirred in the cold for 30 min, quenched with saturated NaHCO₃, extracted with ethyl acetate, dried, concentrated, and purified by chromatography using a gradient of 3-5% of ethyl acetate in hexanes to provide 2.02 g of product

Step 2

To a solution of 2 g of the product of step 1 in 20 mL of MeOH was added 5 mL of AcOH and 300 mg of PtO₂. The mixture was hydrogenated under a balloon. The progress of the reaction was followed by taking NMR spectra of worked-up portions. After overnight stirring another portion of 300 mg of PtO₂ was added and hydrogenation continued for additional 24 h. Catalyst was filtered out, volatiles evaporated, residue re-dissolved in DCM and washed with 1M NaOH solution, saturated NaHCO₃, dried, and evaporated. Column chromatography yielded 1.30 g of cis (6-phenyl-piperidin-2-yl)-methanol and 200 mg of cis (6-cyclohexyl-piperidin-2-yl)-methanol.

Preparation B: Alternate Synthesis of cis (6-phenyl-piperidin-2-yl)-methanol:

Step 1

Treat 6-bromopicolinic acid (1.99 g) in DMF (10 mL) with potassium carbonate (1.40 g) and then methyl iodide (4 mL) at room temperature for 20 h. Dilute the reaction mixture with dichloromethane (60 mL) and filter. Extract the filtrate with brine (twice), dry (MgSO₄), and concentrate in vacuo to give methyl 6-Bromopicolinate as a pale yellow solid (1.75 g).

Step 2

Heat methyl 6-bromopicolinate (0.75 g), phenylboronic acid (0.61 g), tetrakis(triphenylphosphine)palladium (0.19 g) and potassium carbonate (0.75 g) in toluene (20 mL) and methanol (4.5 mL) under reflux for 1 hr. Cool the reaction mixture, dilute with dichloromethane, and filter. Wash the filtrate with water. Concentrate the dried (K₂CO₃) organic solution in vacuo to give an amber residue (0.81 g). Chromatograph this residue on silica gel plates (8, 1000□) using hexane:ethyl acetate 3:1 as eluant to give methyl 6-phenylpicolinate as a colorless oil (0.55 g).

Step 3

Under a hydrogen atmosphere, stir methyl 6-phenylpicolinate (0.55 g) in MeOH (30 mL) and glacial acetic acid (15 mL) in the presence of platinum oxide (0.150 g) for 5 hr. Purge the reaction mixture with nitrogen. Filter and then concentrate the reaction mixture in vacuo to give a yellow oil (0.77 g). Chromatograph this oil on silica gel plates (8, 1000□) using hexane:ethyl acetate 3:1 eluant to give methyl 6-phenylpipecolinate as a colorless oil (0.23 g).

Step 4

Treat methyl 6-phenylipecolinate (0.23 g) in THF (15 mL) with 1M lithium aluminum hydride in ether (10 mL) at room temperature for 2 h. Quench the reaction mixture with EtOAc, the add MgSO₄ and filter. Concentrate the filtrate to give a residue. Chromatograph the residue on silica gel plates (2, 1000 m) using EtOAc:hexane 1:1 as eluant to give (6-phenyl-piperidin-2-yl)-methanol as a white solid (0.06 g).

Preparation C:

Step 1

(a) At 0° C., to a solution of 1.29 g (6.77 mmol) of cis (6-phenyl-piperidin-2-yl)-methanol from Preparation A or Preparation B in 20.0 mL of DCM was added 1.90 mL (13.6 mmol) of triethylamine and 1.84 mL (10.1 mL) of trimethylsilyl trifluoromethanesulfonate. The mixture was stirred for 1 h at ambient temperature, washed with saturated NaHCO₃, dried over Na₂SO₄ and volatiles were evaporated.

(b) The residue was re-dissolved in DCM, treated with 1.90 mL (13.5 mmol) of triethylamine and 2.11 g (10.0 mmol) of 4-chlorobenzenesulfonylchloride. The mixture was stirred for 24 h, washed with 1M HCl, saturated NaHCO₃, and concentrated.

(c) To insure cleavage of TMS ether, the material was dissolved in methanol (5 mL), treated with 1 mL of 1 M HCl, stirred for 30 min, and concentrated. The residue was chromatographed using 10-20% ethyl acetate in hexanes to furnish 1.45 g of 1-(4-Chloro-benzenesulfonyl)-6-phenyl-piperidin-2-yl]-methanol.

Step 2

The product of Step 1 was converted to the title compound according to Step 4 of Example 1, using N-cyclohexylpiperazine at the last stage as the amine. 1H NMR (CDCl₃ 300 MHz) δ 7.86 (2H, d, J=8.2 Hz), 7.57-7.49 (4H, m), 7.36-7.24 (3H, m), 5.24 (1H, d, J=4.9 Hz), 4.34 (1H, q, J=6.2 Hz), 3.68 (1H, dd, J=11.0, 6.5 Hz), 3.58-3.40 (5H, ser.m.), 2.55 (4H, m), 2.37-2.24 (2H, ser.m.), 1.90-1.58 (6H, ser.m.), 1.53-1.36 (3H, ser.m.), 1.30-1.13 (6H, ser.m.); MS (ES) m/e 560.1 (M+H)⁺.

Following procedures similar to those in Example 53, the compounds in Table 4 were prepared. Cis-(6-cyclohexyl-piperidin-2-yl)-methanol, obtained in Preparation A, step 2, was used in Examples 63-66.

TABLE 4

| EX. | | |
|---|---|---|
| No. | COMPOUND | Mass Spec |
| 54 | 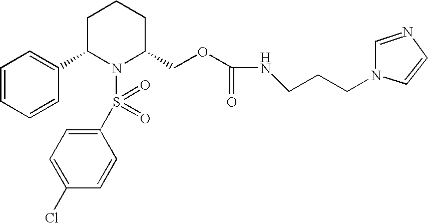 | 517.1 |
| 55 | 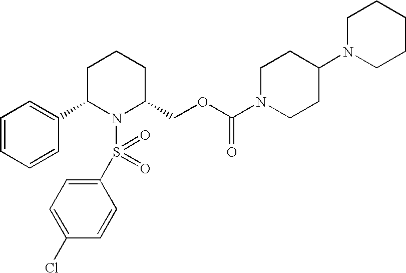 | 560.1 |
| 56 | 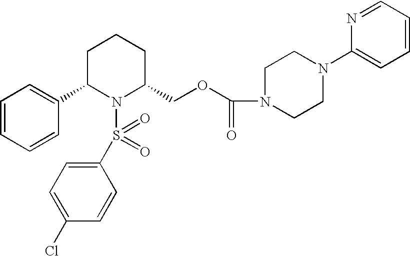 | 555.1 |
| 57 | 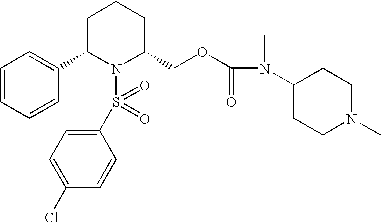 | 520.1 |
| 58 | 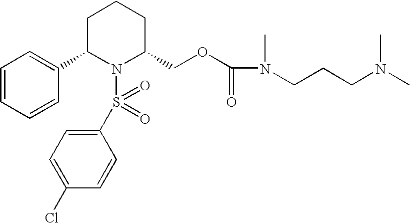 | 508.1 |
| 59 | 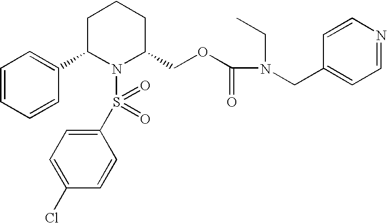 | 528.1 |
| 60 | 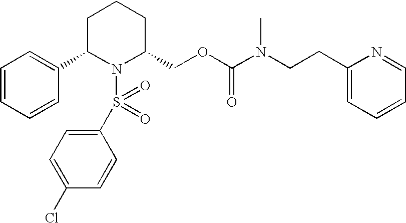 | 528.1 |
| 61 | 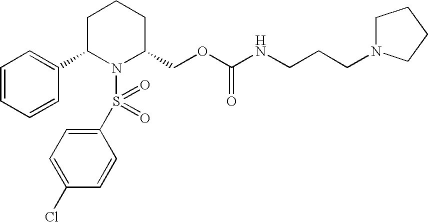 | 520.1 |
| 63 | 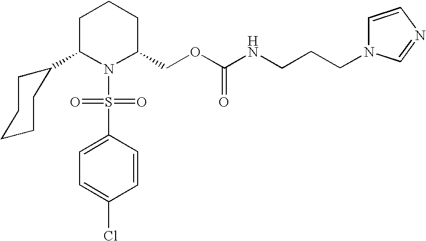 | 523.1 |
| 64 | 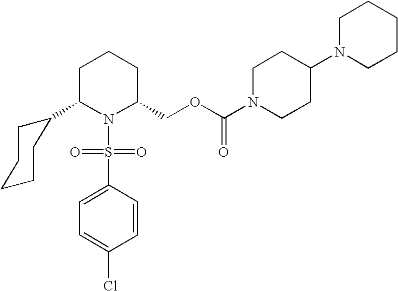 | 566.1 |
| 65 | 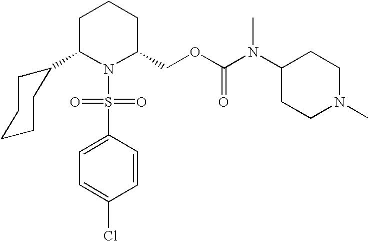 | 526.1 |
| 66 | 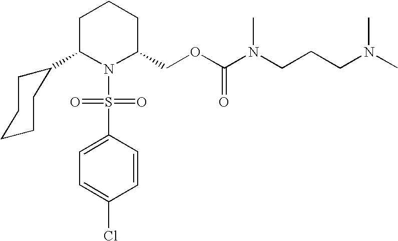 | 514.1 |
| 67 | 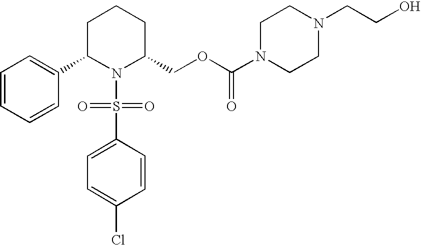 | 522.1 |

NMR data are given in Table 5 below for compounds in Table 4:

TABLE 5

| EX. | | |
|---|---|---|
| No. | COMPOUND | NMR |
| 54 | 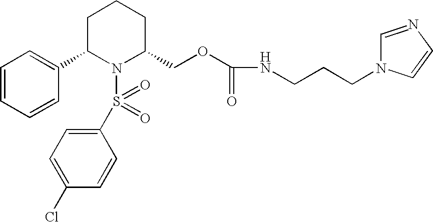 | 1H NMR (CDCl3 300 MHz) δ 7.84(2H, d, J=8.8 Hz), 7.58-7.51(5H, ser.m.), 7.37-7.24(3H, ser.m.), 7.07 (1H, br), 6.96(1H, br), 5.22 (1H, d, J=5.5Hz), 4.91(1H, m), 4.33(1H, m), 4.03(2H, t, J=7.0Hz), 3.75(1H, dd, J=6.0, 11.5Hz), |
| # 3.42(1H, dd, J=6.0, 11.5Hz), 3.27-3.17(1H, m), 3.13-3.05(1H, m), 2.34(1H, d, J=14.8Hz), 1.88(2H, m), 1.68-1.19(5H, ser.m.) | | |
| 55 | 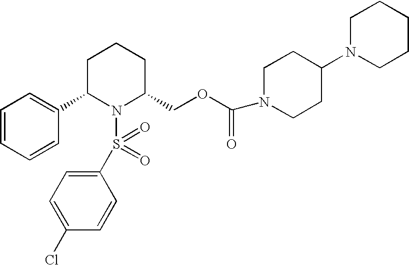 | 1H NMR (CDCl3 300MHz) δ 7.85(2H, d, J=8.8Hz), 7.57-7.50(4H, ser.m.), 7.36-7.23(3H, ser.m.), 5.24(1H, d, J=4.5Hz), 4.38-4.13(3H, ser.m.), 3.70(1H, dd, J=6.0, 11.0Hz), 3.47(1H, s), 3.42 (1H, dd, J=9.0, 11.0Hz), 2.73(1H, br), |
| # 2.53-2.30(5H, ser.m.), 1.94-1.17(16H, ser.m.) | | |
| 57 | 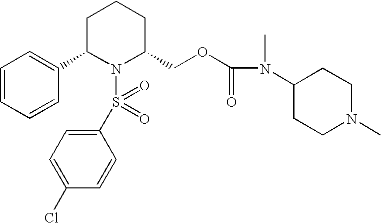 | 1H NMR (CDCl3 300MHz) δ 7.84(2H, d, J=8Hz), 7.59-7.50(4H, m), 7.34-7.26(3H, ser.m.), 5.23(0.5H, br), 5.12 (0.5H, br), 4.59(0.5H, br), 4.47-4.32(1H, m), 4.11 (0.5H, br), 3.71(1H, d, J=10.2Hz), 3.43(2H, t, J=10.5Hz), 3.24(1H, br) |
| 58 | 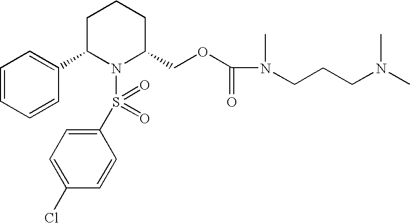 | 1H NMR (CDCl3 300 MHz) δ 7.83(2H, d, J=8Hz), 7.63-7.53(4H, ser.m.), 7.38-7.27(3H, ser.m.), 5.18(1H, m), 4.44(1H, m), 3.86-3.62 3H, ser.m.), 3.56-3.30(2H, ser.m.), 3.00(3H, s), 2.78 (3H, s), 2.73(3H, s) |
| 61 | 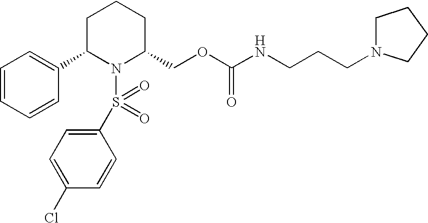 | 1H NMR (CDCl3 300 MHz) δ 7.84(2H, d, J=8.7Hz), 7.56-7.51(4H, ser.m), 7.38 (2H, t, J=7.3Hz), 7.29(1H, d, J=7.3Hz), 5.66(1H, m), 5.20 (1H, d, J=5.1Hz), 4.35 (1H, m), 3.70(1H, dd, J=1 1.2, 7.0Hz), 3.45-3.0 (3H, ser.m), |
| # 3.27-2.96(5H, ser.m), 2.30(1H, d, J=1 4.0 Hz), 2.04-1.2(12H, ser.m) | | |
| 64 | 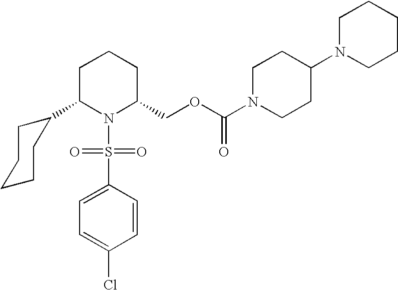 | 1H NMR (CDCl3 300MHz) δ 7.78(2H, d, J=8.5Hz), 7.47(2H, d, J=8.5Hz), 4.29-4.08(5H, ser.m.), 3.68(1H, m), 2.76(2H, m), 2.5-2.35 (5H, ser.m.), 2.10(1H, d, J=12.6Hz), 1.85-0.77(26H, ser.m.) |
| 67 | 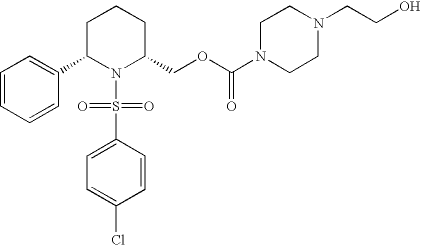 | 1H NMR (CDCl3 300MHz) δ7.85(2H, d), 7.57-7.50(4H, ser.m.), 7.37-7.24(3H, ser.m.), 5.24(1H, d, J=4.5 Hz), 4.35(1H, m), 3.72(1H, dd, J=11.0, 6.0Hz), 3.64 (2H, t, J=5.2Hz), 3.60-3.45 (4H, ser.m.), 3.43(1H, dd, J=11.0, 9.3Hz), |
| # 2.61-2.46 (6H, ser.m.), 2.35(1H, d, J=14.3Hz), 1.73-1.18(6H, ser.m.) | | |

Also prepared were the following compounds:

TABLE 5-A

| Compound | | Retention | |
|---|---|---|---|
| No. | Structure | Time (minutes) | Observed Mass |
| 67-A | 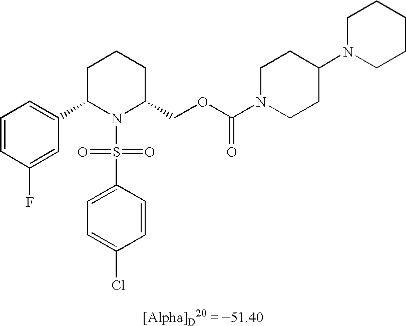 | 5.38 | 578.1 |
| 67-B | 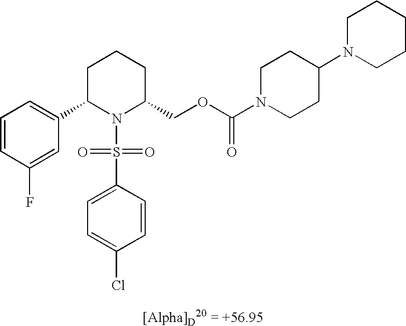 | 5.38 | 578.1 |
| 67-C | 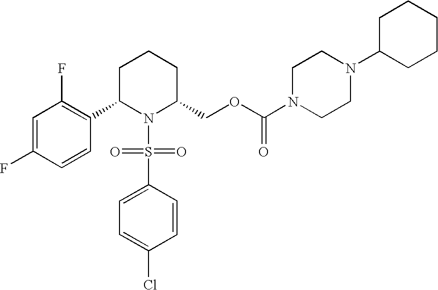 | 5.52 | 596.1 |
| 67-D | 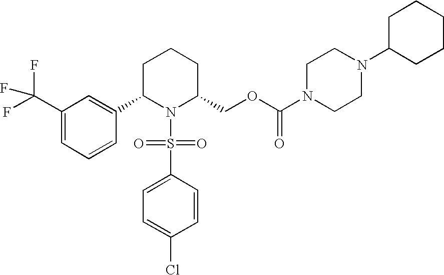 | 5.68 | 628.1 |
| 67-E | 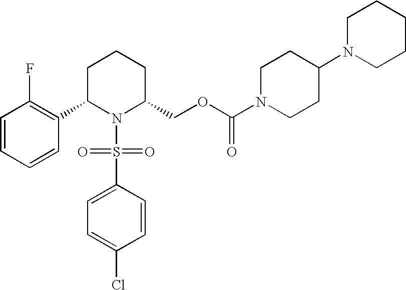 | 5.42 | 578.1 |
| 67-F | 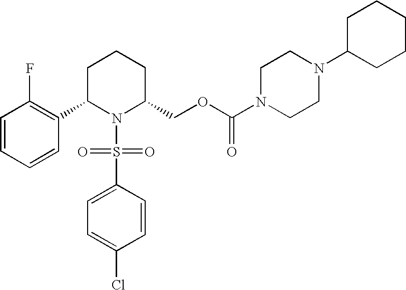 | 5.48 | 578.1 |
| 67-G | 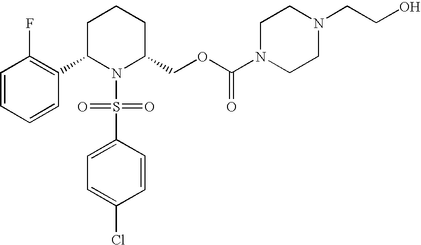 | 4.83 | 540.1 |
| 67-H | 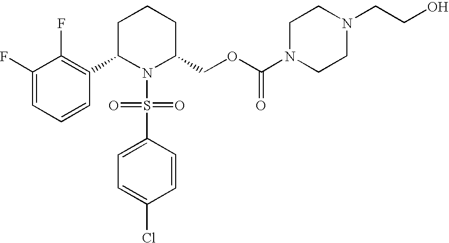 | 4.75 | 558.1 |
| 67-I | 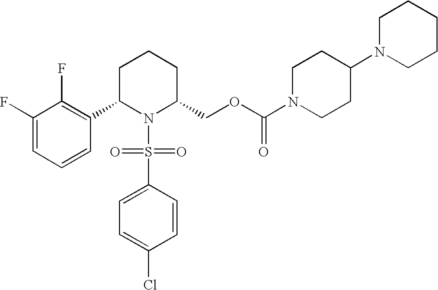 | 5.42 | 596.1 |
| 67-J | 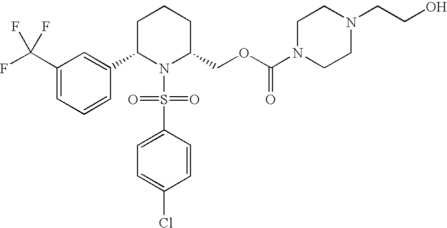 | 5.18 | 590.1 |
| 67-K | 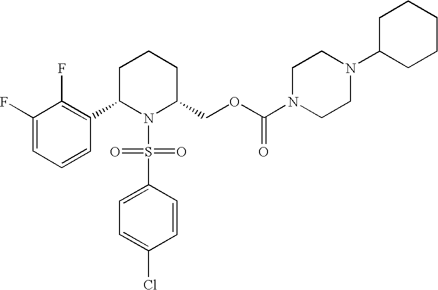 | 5.48 | 596.1 |
| 67-L | 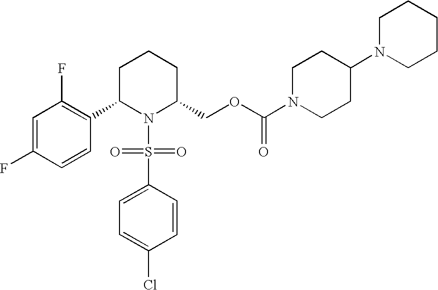 | 5.62 | 596.1 |
| 67-M | 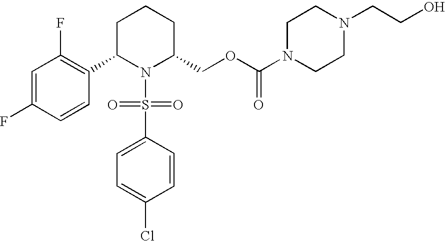 | 4.85 | 558.1 |
| 67-N | 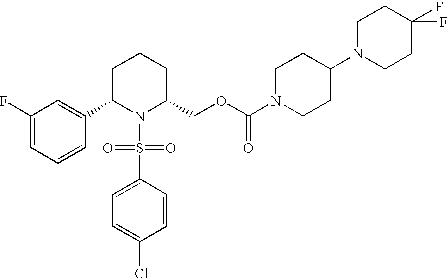 | 5.51 | 614.3 |
| 67-O | 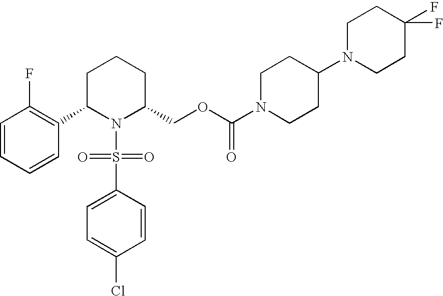 | 5.48 | 614.3 |
| 67-P | 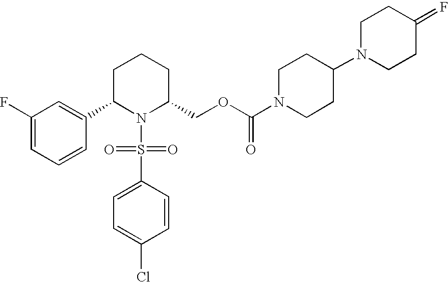 | 5.55 | 590.1 |
| 67-Q | 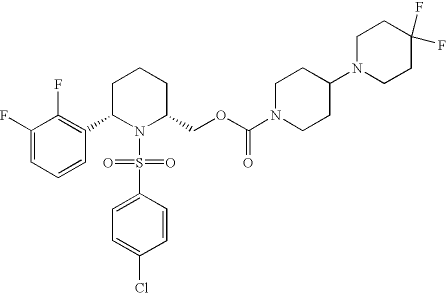 | 5.48 | 632.1 |
| 67-R | 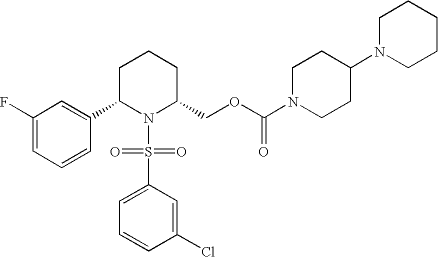 | 5.82 | 578.1 |
| 67-S | 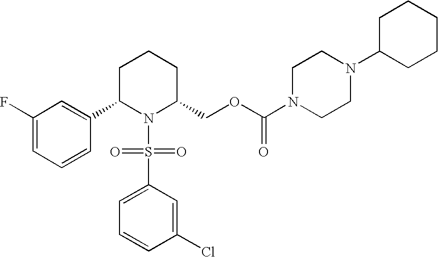 | 5.85 | 578.1 |
| 67-T | 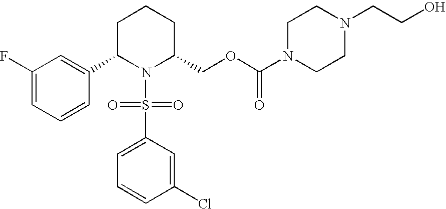 | 5.35 | 540.1 |
| 67-U | 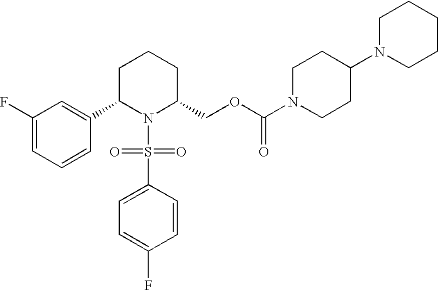 | 5.65 | 562.1 |
| 67-V | 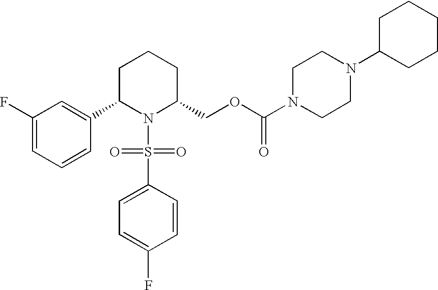 | 5.68 | 562.1 |
| 67-W | 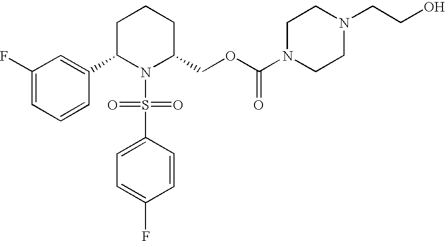 | 5.18 | 524.1 |
| 67-X | 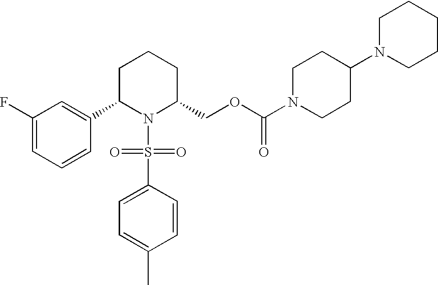 | 5.08 | 558.3 |
| 67-Y | 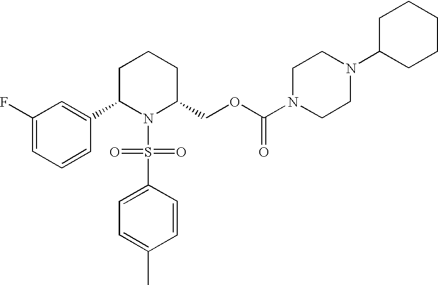 | 5.18 | 558.3 |
| 67-Z | 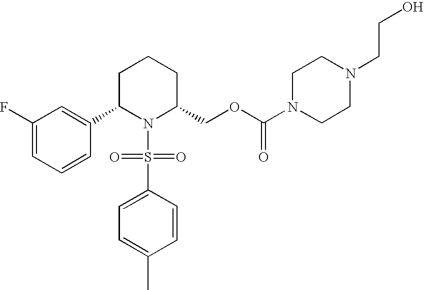 | 4.38 | 520.3 |
| 67-AA | 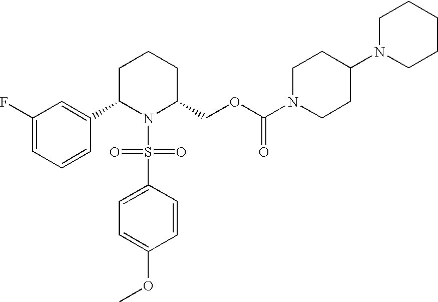 | 5.32 | 574.1 |
| 67-AB | 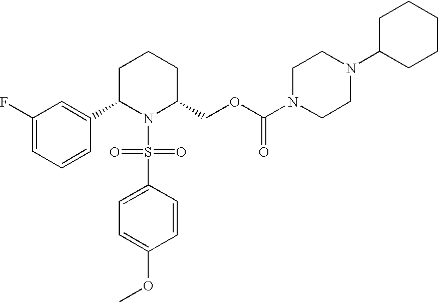 | 5.55 | 574.1 |
| 67-AC | 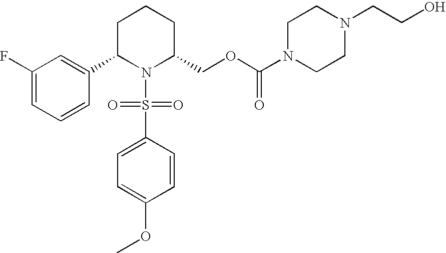 | 4.68 | 536.1 |
| 67-AD | 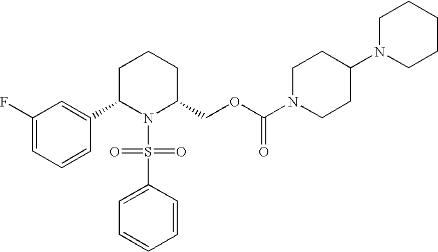 | 5.25 | 544.1 |
| 67-AE | 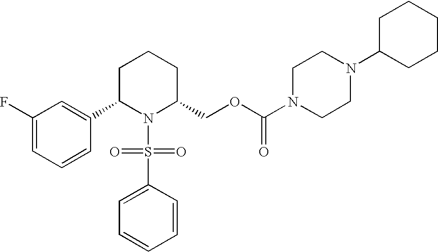 | 5.55 | 544.1 |
| 67-AF | 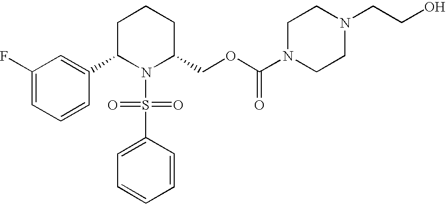 | 4.61 | 506.1 |
| 67-AG | 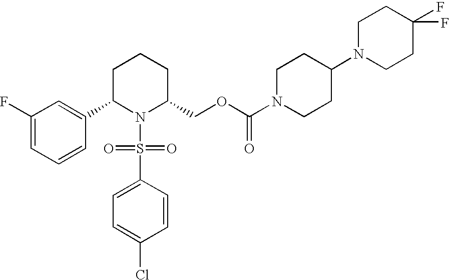 | 5.65 | 608.1 |
| 67-AH | 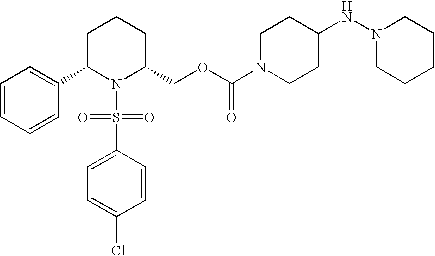 | 5.38 | 575.1 |
| 67-AI | 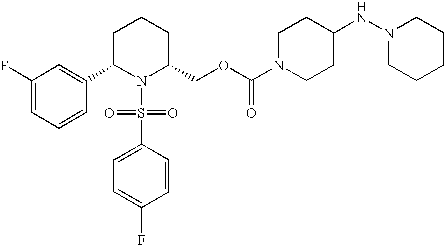 | 5.25 | 577.1 |
| 67-AJ | 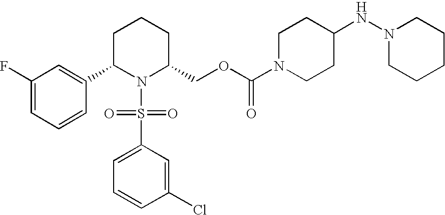 | 5.38 | 593.1 |
| 67-AK | 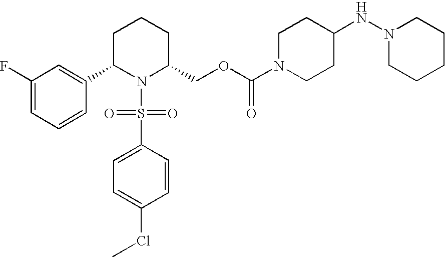 | 5.22 | 589.1 |
| 67-AL | 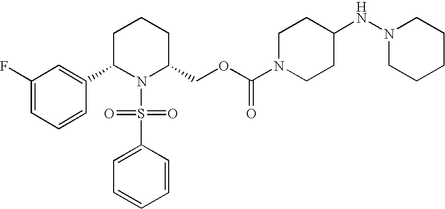 | 5.15 | 559.1 |
| 67-AM | 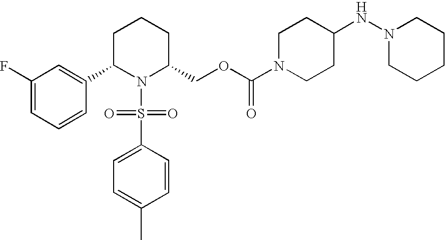 | 5.35 | 573.1 |
| 67-AN | 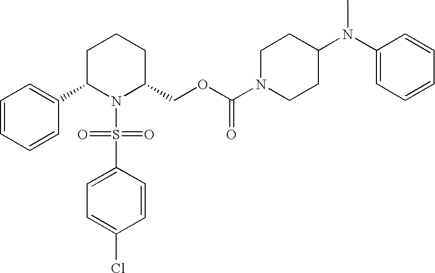 | 5.01 | 582.3 |
| 67-AO | 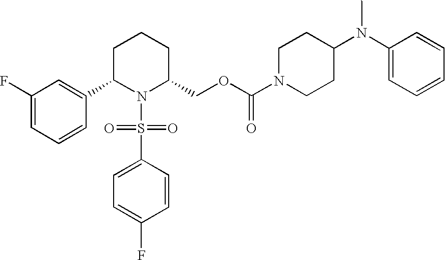 | 4.85 | 584.3 |
| 67-AP | 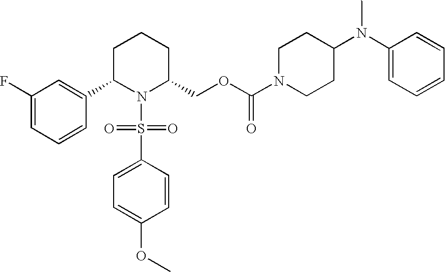 | 4.85 | 596.3 |
| 67-AQ | 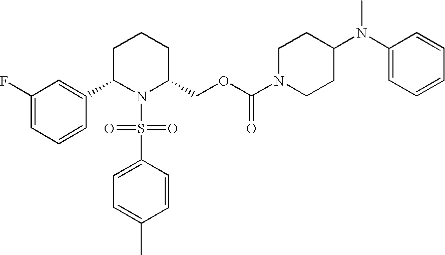 | 5.01 | 580.3 |
| 67-AR | 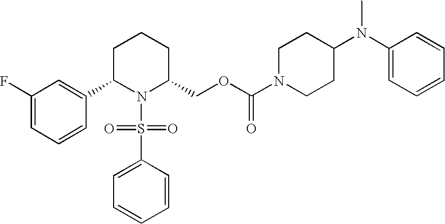 | 4.78 | 566.3 |
| 67-AS 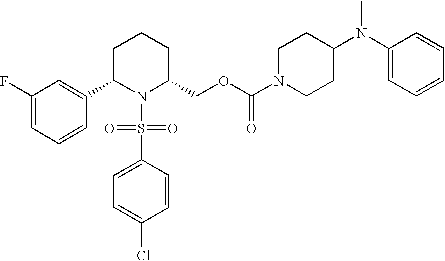 | 5.52 | 600.1 | |
| 67-AT | 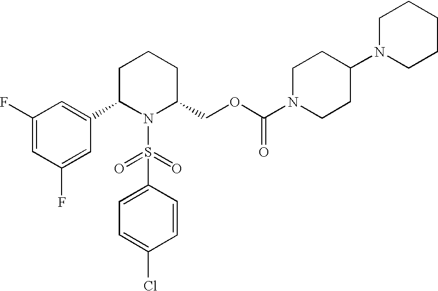 | 5.52 | 596.1 |
| 67-AV | 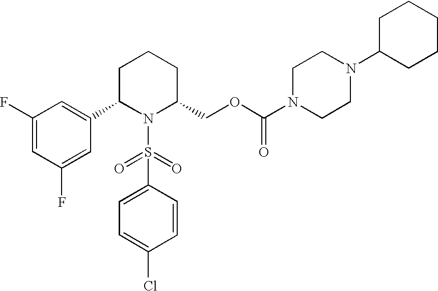 | 5.52 | 596.1 |
| 67-AW | 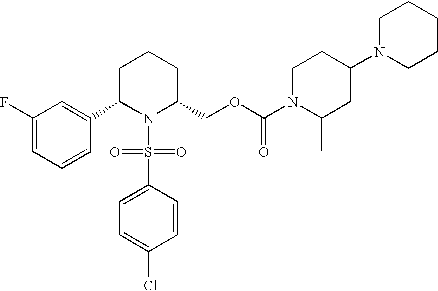 | 5.85 | 578.1 |
| 67-AX | 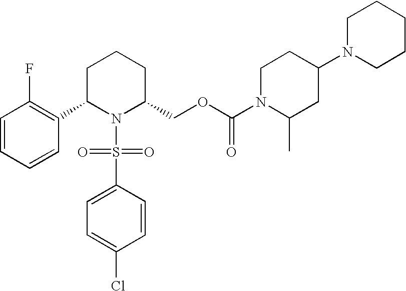 | 5.85 | 578.1 |
| 67-AY | 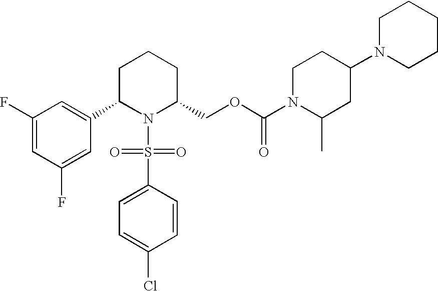 | 5.82 | 596.1 |
| 67-AZ | 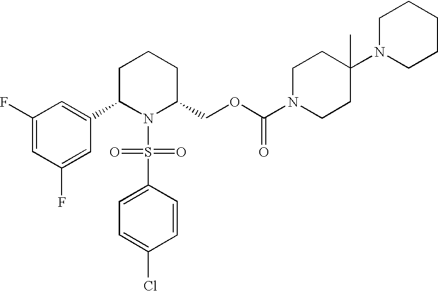 | 545 | 610.1 |
| 67-BA | 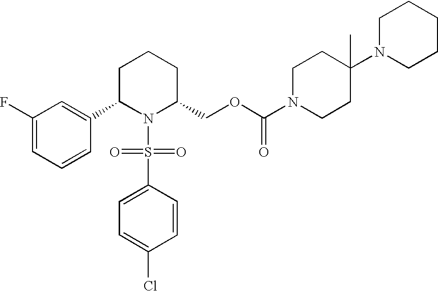 | 5.92 | 592.1 |
| 67-BB | 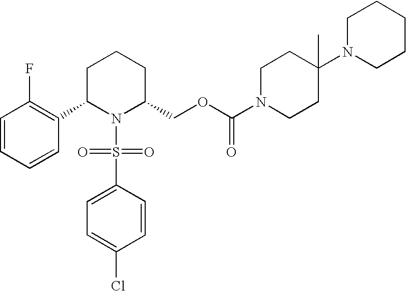 | 5.88 | 592.1 |
| 67-BC | 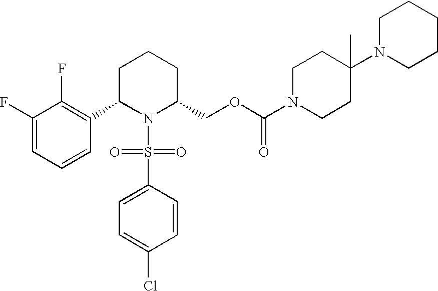 | 5.92 | 610.1 |
| 67-BD | 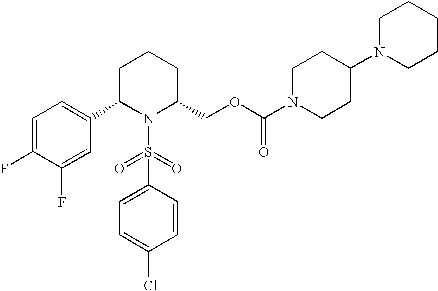 | 5.72 | 596.1 |
| 67-BE | 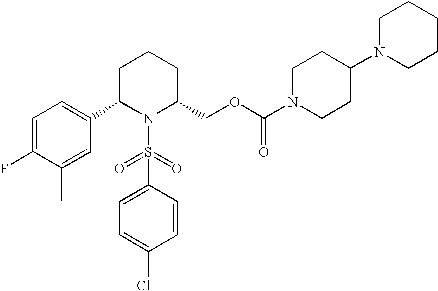 | 5.92 | 592.1 |
| 67-BF | 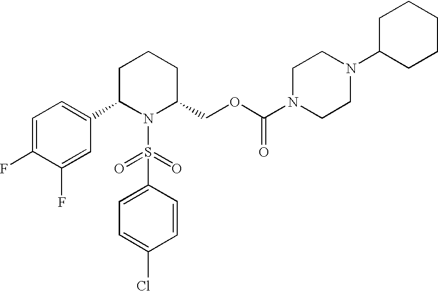 | 5.78 | 596.1 |
| 67-BG | 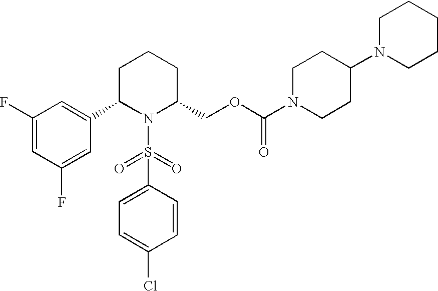 | 5.42 | 596.1 |
| 67-BH | 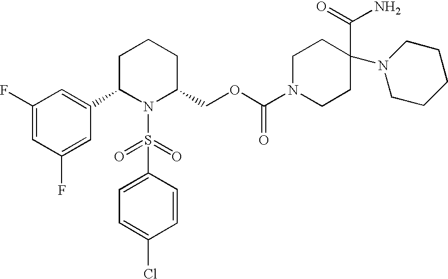 | 5.35 | 639.0 |
| 67-BI | 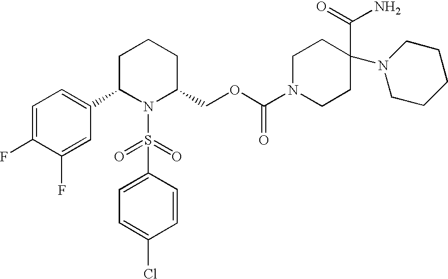 | 5.15 | 639.2 |
| 67-BJ | 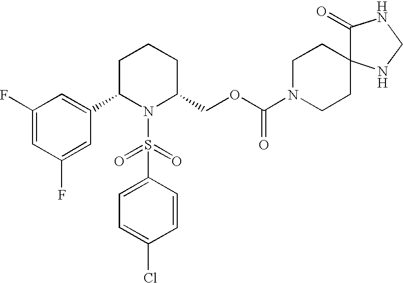 | 4.65 | 583.1 |
| 67-BK | 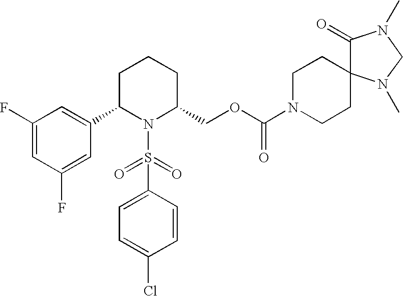 | 5.22 | 611.1 |
| 67-BL | 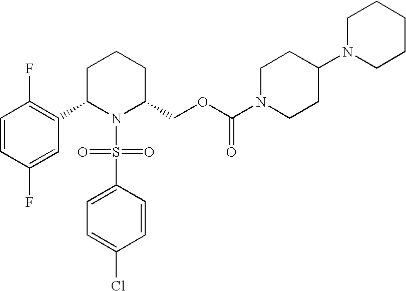 | 5.00 | 596.1 |
| 67-BM | 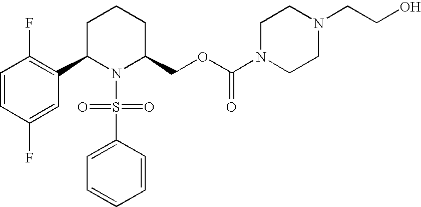 | 4.50 | 558.1 |
| 67-BN | 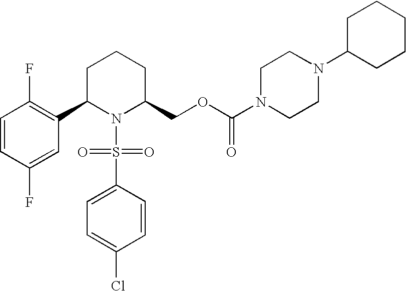 | 5.30 | 596.1 |
| 67-BO | 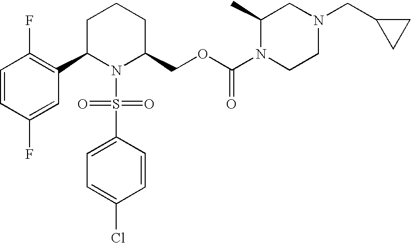 | 5.00 | 582.1 |
| 67-BP | 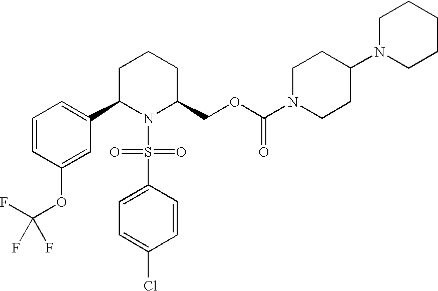 | 5.50 | 644.2 |
| 67-BQ | 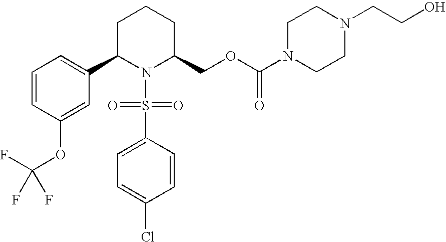 | 5.00 | 606.1 |
| 67-BR | 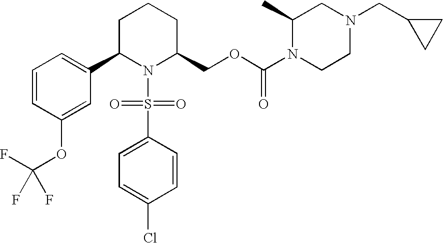 | 5.30 | 631.1 |
| 67-BS | 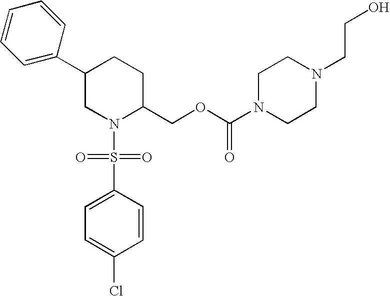 | 4.85 | 522.1 |

### Example 68

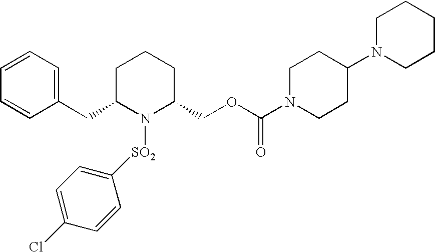

Step 1

(a) A solution of 1.00 g (4.29 mmol) of 2,6-dibromopyridine in a mixture of 20 mL of ether and 20 mL of THF was cooled to −78° C. (becomes turbid due to partial precipitation). To this was added drop-wise 1.86 mL (4.29 mmol) of 2.3 M BuLi, and the reaction was stirred for 5 min.

(b) Benzaldehyde (456 mg, 4.3 mmol) was added drop-wise to the above mixture, and the reaction was stirred in the cold for 15 min, quenched with saturated NaHCO₃, extracted with ethyl acetate, dried, concentrated. The residue was purified by chromatography using a gradient of 10-30% of ethyl acetate in hexane as solvent to give 0.85 g of oily product.

(c) A mixture of the above product, 5 ml of triethylsilane, 5 mL of TFA and 5 mL of DCM was heated at reflux over a period of 36 h. After evaporating most of the volatiles, the residue was redissolved in DCM, washed with 1 M NaOH, dried, concentrated, and purified by chromatography using 5% ethyl acetate in hexanes. Obtained 0.55 g of the product.

Step 2

The product of step 1 was converted to the target compound using conditions described in Example 53, Preparations A and C. 1H NMR (CDCl3 300 MHz) δ 7.75 (2H, d, J=8.8 Hz), 7.44 (2H, d, J=8.8 Hz), 7.33-7.19 (5H, ser.m.), 4.42-4.22 (4H, ser.m.), 4.14 (1H, m), 3.98 (1H, m), 3.09 (1H, dd, J=12.0, 2.7 Hz), 2.90 (1H, t, J=12.0 Hz), 2.78 (2H, br), 2.51-2.37 (5H, ser.m.), 1.84-1.27 (16H, ser.m.); MS (ES) m/e 574.1 (M+H)⁺.

Following procedures similar to Example 68 the compounds in Table 6 were prepared.

TABLE 6

| EX | | Mass |
|---|---|---|
| No. | COMPOUND | Spec |
| 69 | 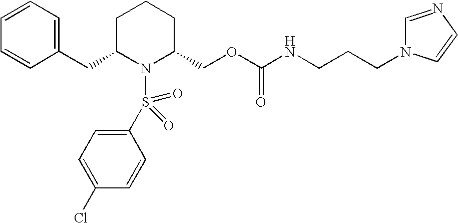 | 531.1 |
| 71 | 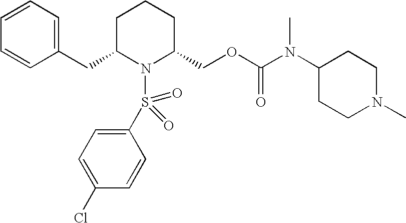 | 534.1 |
| 72 | 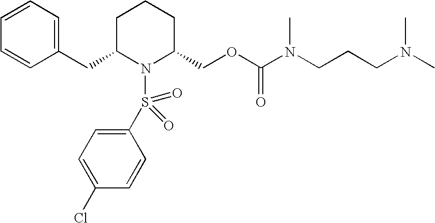 | 522.1 |
| 73 | 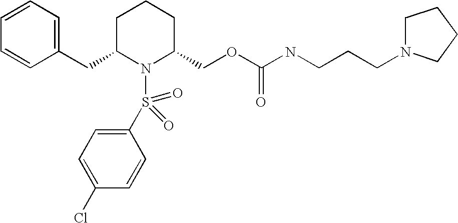 | 534.1 |
| 74 | 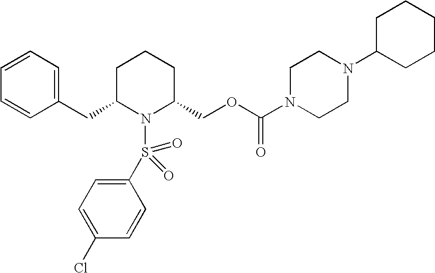 | 574.1 |

Also prepared were the following compounds:

TABLE 6-A

| | | RETENTION | |
|---|---|---|---|
| EXAMPLE | | TIME | OBSERVED |
| NO. | STRUCTURE | (minutes) | MASS |
| 74-A | 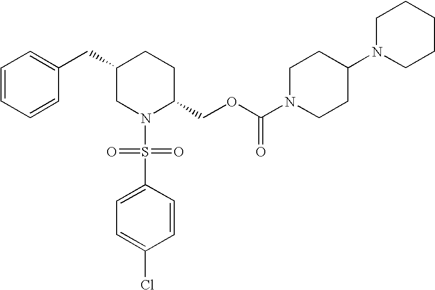 | 5.35 | 574.3 |
| 74-B | 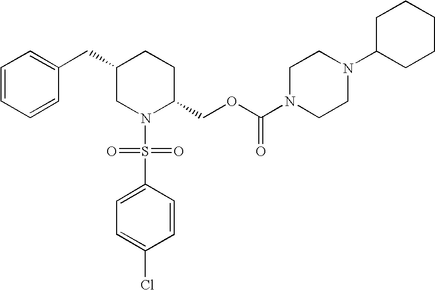 | 5.38 | 574.3 |
| 74-C | 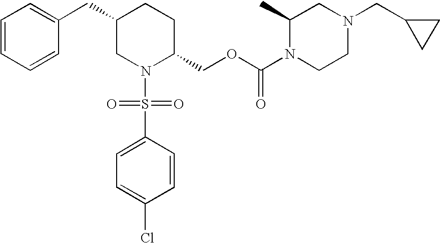 | 5.05 | 560.3 |

### Example 75

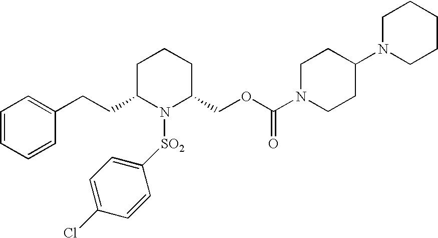

Step 1

To a solution of 5.0 g (21.4 mmol) of 2,6-dibromopyridine in 50.0 mL of DCM was added 5.6 mL (40 mmol) of triethylamine, 701 mg (1 mmol) of Pd(PPh₃)₄Cl₂, 95 mg (0.5 mmol) of CuI, and a mixture of phenylacetylene in 20.0 mL of DCM. The dark mixture was stirred overnight, washed with concentrated ammonium hydroxide, dried, concentrated, and chromatographed. Fractions containing the desired product of mono-substitution of bromine were identified by MS (m/z=258.1), yield 2.41 g.

Step 2

The product of step 1 was converted to the target compound using conditions described in Example 53, Preparations A and C. ¹H NMR (CDCl3 300 MHz) δ 7.73 (2H, d, J=8.8 Hz), 7.45 (2H, d, J=8.8 Hz), 7.31-7.16 (5H, ser.m.), 4.30 (4H, m), 4.13 (1H, m), 3.97 (1H, m), 2.73 (4H, m), 2.42 (5H, m), 2.04 (1H, m), 1.78-1.15 (17H, ser.m.); MS (ES) m/e 588.1 (M+H)⁺.

Following procedures similar to those of Example 75 the compounds in Table 7 were prepared.

TABLE 7

| EX | | Mass |
|---|---|---|
| No. | COMPOUND | Spec |
| 76 | 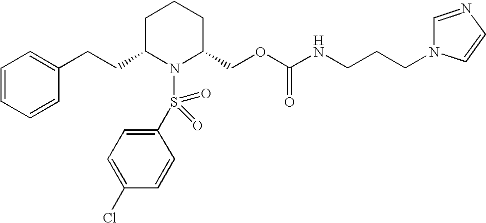 | 545.1 |
| 78 | 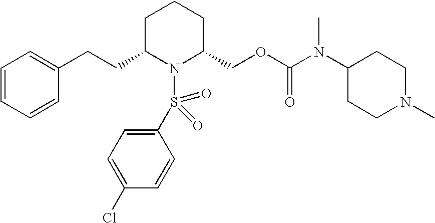 | 548.1 |
| 79 | 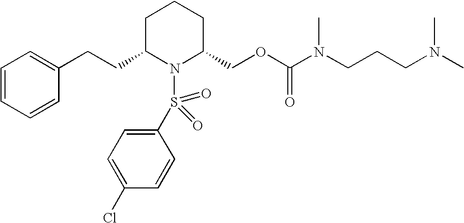 | 536.1 |
| 80 | 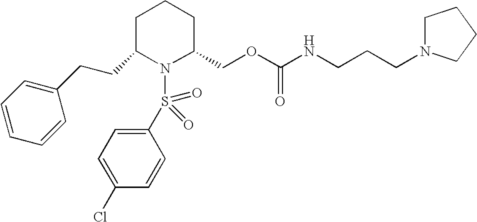 | 548.1 |
| 81 | 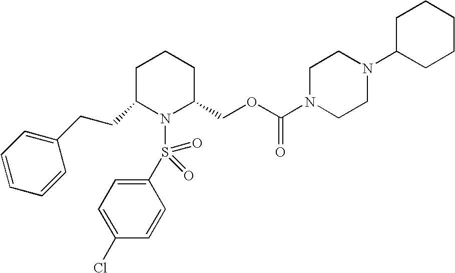 | 588.1 |

### Example 82

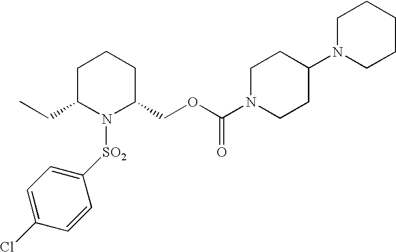

Step 1

To a solution of 5.0 g (21.2 mmol) of 2,6-dibromopyridine in THF at −78° C. was added 9.2 mL (21 mmol) of 2.3 M solution of n-BuLi in hexanes, followed by 2.3 mL (30 mmol) of DMF. The mixture was stirred for 45 min in the cold, quenched with saturated NaHCO₃, extracted with ethyl acetate and the product purified by column chromatography (3% ethyl acetate in hexanes) to furnish 1.13 g of 2-bromo-6-formylpyridine.

Step 2

(a) A mixture containing 750 mg (4.05 mmol) of product of step 1, 1.41 g (4.46 mmol) of vinyltributyltin, 231 mg (0.2 mmol) of Pd(PPh₃)₄, and 5.0 mL of DMF was heated for 12 h at 90° C. The volatiles were evaporated, and the residue purified by chromatography (3-5% ethyl acetate in hexanes) to furnish 360 mg of 2-formyl-6-vilylpyridine.

(b) The above product was hydrogenated at 50 psi over catalytic PtO₂ using 1:3 mixture of AcOH and MeOH as solvent to furnish 87 mg of reduced product

Step 3

The product of step 2 was converted to the target compound using conditions described in Example 53, Preparations C. ¹H NMR (CDCl3 300 MHz) δ 7.77 (2H, d, J=8 Hz), 7.47 (2H, d, J=8 Hz), 4.29-4.22 (4H, ser.m.), 4.05 (1H, m), 3.79 (1H, d, 2.77 (2H, br), 2.50-2.37 (5H, ser.m.), 1.83-1.70 (6H, ser.m.), 1.62-1.10 (12H, ser.m.), 0.96 (3H, t, J=7.3 Hz); MS (ES) m/e 512.1 (M+H)⁺.

Following procedures similar to those of Example 82 the compounds in Table 8 were prepared.

TABLE 8

| EX | | Mass |
|---|---|---|
| No. | COMPOUND | Spec |
| 83 | 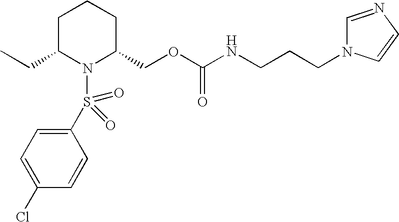 | 469.1 |
| 85 | 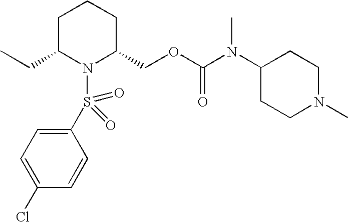 | 472.1 |
| 86 | 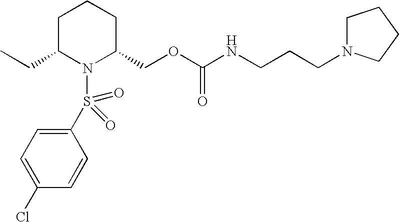 | 472.1 |
| 87 | 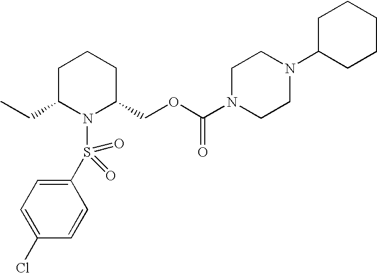 | 512.1 |

### Example 88

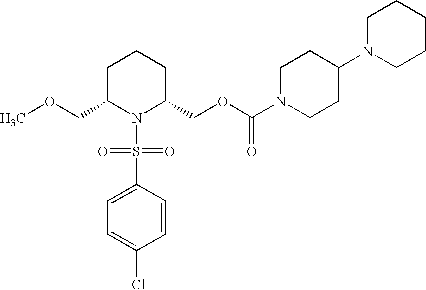

Step 1

To a solution of 2,6-pyridinedicarboxylate methyl ester (19.52 g; 100 mmol) in ice-cooled anhydrous methanol (300 ml) is added sodium borohydride (3.03 g; 80 mmol) portion-wise then the reaction is stirred 30 min at room temperature. Another 1.0 g of sodium borohydride is added to the mixture and the reaction is stirred an additional 30 minutes. After concentration, the crude is diluted with water and CH₂Cl₂ and extracted with CH₂Cl₂. Combined organic layers are dried over Na₂SO₄, concentrated, and the residue is subjected to flash-chromatography over silica gel (eluting CH₂Cl₂/MeOH 95:5) to give 11.09 g (66%) of alcohol, as a white solid.

Step 2

To a solution of alcohol (9.00 g; 53.8 mmol) in anhydrous THF (200 mL) at 0 C is added NaH 60% in mineral oil (2.60 g; 64.6 mmol) followed by dimethylsulfate (6.60 ml; 70 mmol) and the reaction is stirred 2 h at 35 C. After concentration, the crude is diluted with water and extracted with CH₂Cl₂. Combined organic layers are dried over Na2SO4, concentrated, and the residue is subjected to flash-chromatography over silica gel (eluting CH₂Cl₂/MeOH 95:5). The purified product is dissolved in CH₂Cl₂/MeOH, treated with an excess of 1 N HCl in Et2O and concentrated to provide 11.5 g (98%) of pyridine intermediate, as a hydrochloride salt.

Step 3

A mixture of pyridine intermediate (11.50 g; 52.8 mmol) and platinum (IV) oxide (1 g) in ethanol is hydrogenated 16 h at 40 psi, filtered over Celite and concentrated to provide 11.60 g of crude piperidine amine, as a white solid.

Step 4

To a suspension of piperidine amine (11.60 g; 52.1 mmol) in anhydrous THF (50 ml) at 0 C is slowly added lithium aluminum hydride 1 N in THF (200 ml; 200 mmol), then the reaction is allowed to warm to room temperature and stirred an additional 1 h. The reaction is quenched with an excess of AcOEt, diluted with 0.5 N aqueous NaOH solution, and extracted with AcOEt and CH₂Cl₂. Combined organic layers are dried over Na₂SO₄ and concentrated to provide 8.3 g of crude piperidine alcohol, as an oil.

Step 5

A solution of piperidine alcohol (8.3 g; 52.1 mmol), tert-butyldimethylsilyl chloride (8.6 g; 57.3 mmol) and triethylamine (8.7 ml; 62.5 mmol) in anhydrous 1,2-dichloroethane (100 ml) is stirred 16 h at 60 C. The reaction mixture is diluted with 0.5 N aqueous NaOH solution and extracted with CH₂Cl₂. Combined organic layers are dried over Na₂SO₄, concentrated, and the residue is subjected to flash-chromatography over silica gel (eluting CH₂Cl₂/AcOEt 95:5 to 70:30) to provide 5.0 g (35%) of O-protected piperidine, as an oil.

Step 6

A solution of O-protected piperidine (2.50 g; 9.14 mmol), 4-chlorobenzenesulfonyl chloride (2.90 g; 13.7 mmol) and triethylamine (1.53 ml; 11 mmol) in anhydrous 1,2-dichloroethane (25 ml) is stirred 3 h at 60 C then overnight at room temperature. The reaction mixture is diluted with 0.5 N aqueous NaOH solution and extracted with CH2Cl2. Combined organic layers are dried over Na₂SO₄, concentrated, and the residue is subjected to flash-chromatography over silica gel (eluting CH₂Cl₂) to provide 3.72 g (90%) of O-protected sulfonamide, as an oil.

Step 7

To a solution of O-protected sulfonamide (3.70 g; 8.3 mmol) in anhydrous THF (50 ml) is added TBAF 1 N in THF (16.6 ml; 16.6 mmol) and the reaction is stirred overnight at room temperature. After concentration, the crude is diluted with 5% NaHCO₃ aqueous solution and extracted with CH₂Cl₂. Combined organic layers are dried over Na₂SO₄, concentrated, and the residue is subjected to flash-chromatography over silica gel (eluting CH2Cl2) to give 2.50 g (93%) of sulfonamide alcohol, as an oil: ¹H-NMR (300 MHz, CDCl₃) δ 7.79 (d, J=8.8 Hz, 2H), 7.47 (d, J=8.8 Hz, 2H), 4.24 (m, 1H), 4.09 (m, 1H), 3.40-3.70 (m, 4H), 3.37 (s, 3H), 1.40-1.70 (m, 3H), 1.20-1.40 (m, 3H); HRMS (MH⁺) 334.0883.

Step 8

To a solution of sulfonamide alcohol (2.50 g; 7.50 mmol) and p-nitrophenyl chloroformate (1.70 g; 8.25 mmol) in anhydrous THF (30 ml) is slowly added triethylamine (1.20 ml; 8.25 mmol) and the reaction is stirred overnight at room temperature. After concentration, the residue is subjected to flash-chromatography over silica gel (eluting Hexanes/AcOEt 90:10) to give 3.70 g (99%) of sulfonamide p-nitrophenylcarbonate, as a foam.

Step 9

A solution of sulfonamide p-nitrophenylcarbonate (50 mg; 0.10 mmol) and 4-piperidinopiperidine (84 mg; 0.50 mmol) in 1,2-dichloroethane (1 ml) is stirred overnight at room temperature. The reaction mixture is diluted with 0.5 N aqueous NaOH solution and CH₂Cl₂ and the organic layer is directly subjected to preparative chromatography over silica gel (eluting CH2Cl2) then treated with dry 1 N HCl in Et₂O to provide 7 mg of product: ¹H-NMR (300 MHz, CDCl₃) δ 7.76 (d, J=8.8 Hz, 2H), 7.46 (d, J=8.8 Hz, 2H), 4.15-4.35 (m, 4H), 3.85-4.00 (m, 2H), 3.40-3.55 (m, 3H), 3.34 (s, 3H), 2.65-2.90 (m, 2H), 2.10-2.60 (m, 6H), 1.80-1.90 (br d, 2H), 1.00-1.80 (m, 12H); HRMS (MH⁺) 528.2305.

Following the procedures similar to those in Example 88 the compounds in Table 9 were prepared.

TABLE 9

| EX | | High Res. |
|---|---|---|
| No. | COMPOUND | Mass Spec |
| 89 | 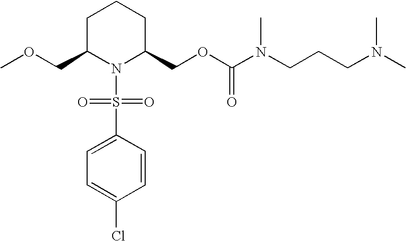 | 476.1985 |
| 90 | 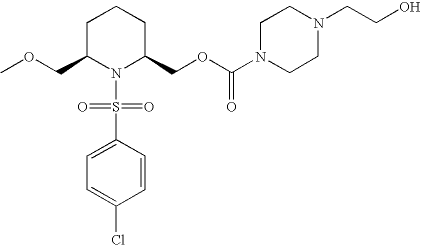 | 490.1776 |
| 91 | 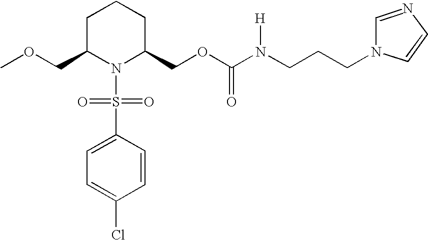 | 485.1630 |
| 92 | 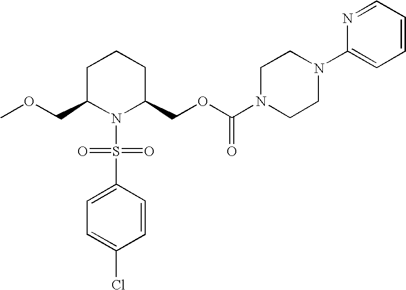 | 523.1792 |
| 93 | 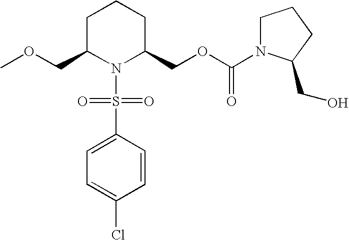 | 460.1440 |
| 94 | 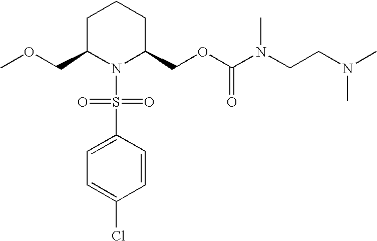 | 462.1656 |
| 95 | 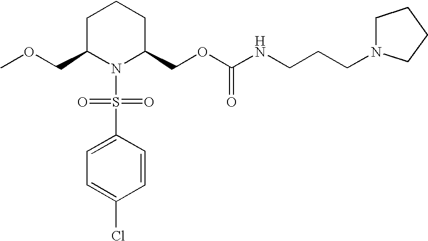 | 488.1989 |
| 96 | 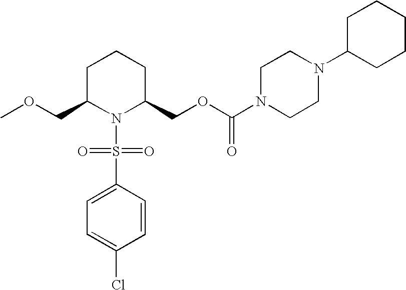 | 528.2304 |
| 97 | 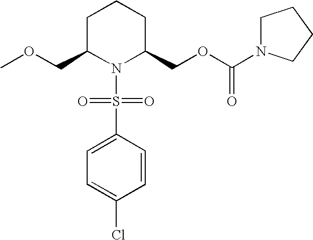 | 431.1413 |
| 98 | 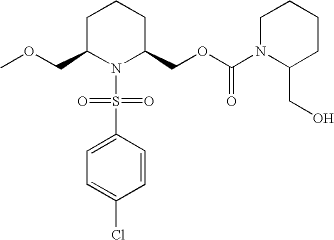 | 475.1661 |
| 99 | 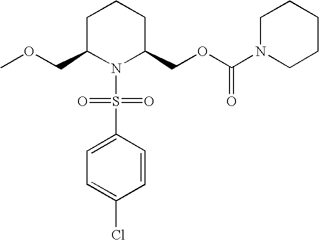 | 445.1568 |
| 101 | 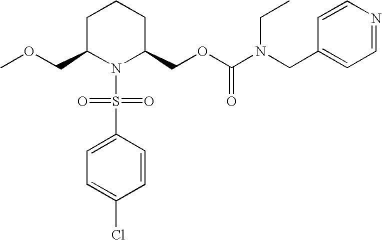 | 496.1679 |
| 102 | 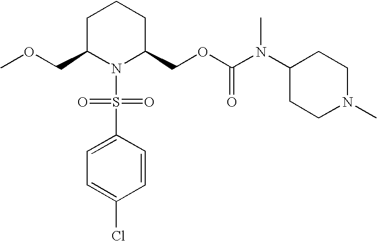 | 488.1986 |
| 103 | 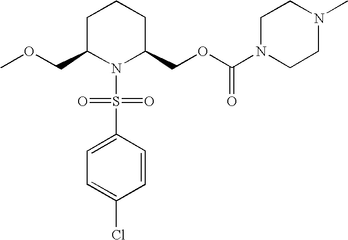 | 460.1677 |
| 104 | 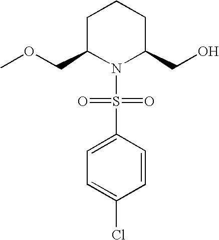 | 334.0883 |

NMR data for compounds in Table 9 are given in Table 10.

TABLE 10

| EX | | |
|---|---|---|
| No. | COMPOUND | NMR (δ) |
| 92 | 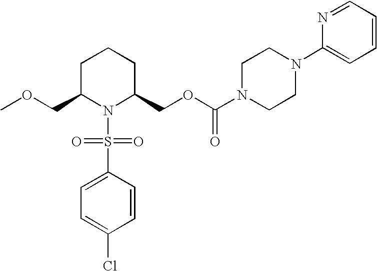 | 8.19 (d, J=3.9 Hz, 1H), 7.77 (d, J=8.8 Hz, 2H), 7.40-7.55 (m, 3H), 6.60-6.75 (m, 2H), 4.20-4.35 (m, 2H), 3.95-4.05 (m, 2H), 3.40-3.75 (m, 10H), 3.35 (s, 3H), 1.50-1.80 (m, 3H), 1.05-1.40 (m, 3H) |
| 94 | 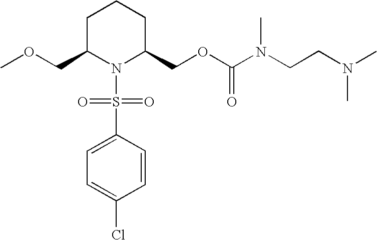 | 7.78 (d, J =8.8 Hz, 2H), 7.46 (d, J=8.8 Hz, 2H), 4.15-4.35 (m, 2H), 3.90-4.05 (n, 2H), 3.35-3.60 (m, 4H), 3.35 (s, 3H), 2.96 (s, 3H), 2.45-2.60 (m, 2H), 2.31 (s, 3H), 2.28 (s, 3H), 1.45-1.80 (m, 3H), 1.20-1.40 (m, 2H), 1.13 (m, 1H) |
| 96 | 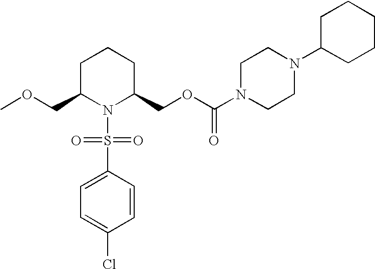 | 7.76 (d, J=8.8 Hz, 2H), 7.46 (d, J=8.8 Hz, 2H), 4.15-4.35 (m, 2H), 3.90-4.00 (m, 2H), 3.40-3.60 (m, 6H), 3.34 (s, 3H), 2.45-2.65 (m, 4H), 2.29 (m, 1H), 1.45-1.90 (m, 6H), 1.00-1.40(m, 1OH) |
| 100 | 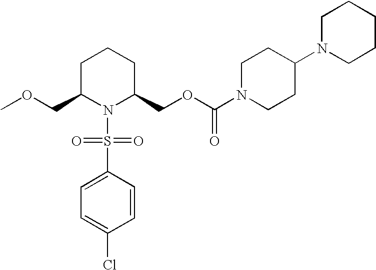 | 7.76 (d, J=8.8 Hz, 2H), 7.46 (d, J=8.8 Hz, 2H), 4.15-4.35 (m, 4H), 3.85-4.00 (m, 2H), 3.40-3.55 (m, 3H), 3.34 (s, 3H), 2.65-2.90 (m, 2H), 2.10-2.60 (m, 6H), 1.80-1.90 (br d, 2H), 1.00-1.80 (m, 12H) |
| 104 | 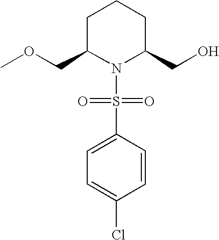 | 7.79 (d, J=8.8 Hz, 2H), 7.47 (d, J=8.8 Hz, 2H), 4.24 (m, 1H), 4.09 (m, 1H), 3.40-3.70 (m, 4H), 3.37 (s, 3H), 1.40-1.70 (m, 3H), 1.20-1.40 (m, 3H) |

### Example 105

Preparation A

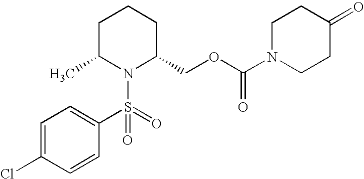

Step 1

Treat the 4-nitrophenylcarbonate product of Example 1, step 4-a (1.26 g) in methanol (50 mL) with 1,4-dioxa-8-azaspiro[4.5]decane (0.76 mL) and stir the resulting mixture at room temperature for 66 h. Concentrate the reaction mixture in vacuo and partition the residue between ethyl acetate/10% sodium hydroxide solution. Extract the ethyl acetate (EtOAc) solution with water, and then brine. Concentrate the dried (MgSO4) EtOAc solution in vacuo to give a pale yellow oil (1.26 g). Chromatograph this oil on silica gel plates (8, 1000μ) using EtOAc:hexane 1:3 as eluant (two elutions) to give the title compound, as a colorless oil (1.11 g).

Step 2

To the product of step 1 (1.10 g) in dichloromethane (20 mL), add 40% trifluoroacetic acid (TFA) in water (8 mL), and stir the resulting mixture for 4 hr. Add additional 40% TFA in water (6 mL). After 2 h, add 40% TFA in water (3 ml). Stir the resulting mixture at room temperature for 18 hr. Separate the reaction mixture. Partition the dichloromethane solution water and then sodium bicarbonate solution. Concentrate the dried (MgSO₄) dichloromethane solution in vacuo to give a colorless foam. Chromatograph this foam on silica gel plates (8, 1000μ) using EtOAc:hexane 1:3) as eluant to give the title compound (0.80 g).

Preparation B

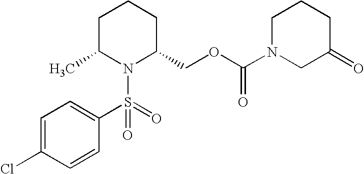

Step 1

Treat the 4-nitrophenylcarbonate product of Example 1, step 4-a (0.100 g) in methanol (55 mL) with 3-hydroxypiperidine (0.060 g, liberated from the hydrochloride salt) and stir the resulting mixture at room temperature for 24 h. Concentrate the reaction mixture in vacuo and partition the residue between ethyl acetate/10% sodium hydroxide solution. Extract the ethyl acetate (EtOAc) solution with water, and then brine. Concentrate the dried (MgSO₄) EtOAc solution in vacuo to give the title compound, as a colorless oil (0.10 g).

Step 2

Treat the product from step 1, in acetone (5 mL) with Jones Reagent (0.40 mL) for 40 min at room temperature. Quench the reaction mixture with MeOH (2mL), filter, and dilute with dichloromethane. Extract the organic mixture with brine. Concentrate the dried (MgSO4) solution in vacuo to a residue (0.070 g). Chromatograph this residue on silica gel plates (1, 1000μ) using EtOAc:hexane 1:3 as eluant to give the title compound (0.040 g).

Preparation C

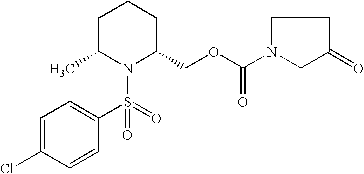

Follow essentially the same procedure as Preparation B, except start with 3-hydroxypyrrolidine (0.060 g) to give the title compound (0.030 g).

Preparation D

Following the procedure described below, the compounds in Table 11 are prepared from the appropriate ketones and amines. The ketones and amines used will be apparent to those skilled in the art from the compounds in Table 11.

Using Bohdan Miniblocks (6 mL cartridge), dispense ketones from Preparation A, B or C (0.010 g) in MeOH:AcOH 9:1 (1 mL). Add amines (1.2 equiv) followed by MP-cyanoborohydride resin (˜2 equiv, 20 to 30 mg., 2.37 mmol/g, Argonaut). Shake the resulting mixture at room temperature for 20 hr. Add PS-isocyanate resin (50-60 mg, 4 equiv. 1.44 mmol/1 g, Argonaut). After 4 h, add additional PS-isocyanate resin (90-100 mg) and leave shaking overnight. Filter from Bohdan block to block and wash the residue with MeOH (1 mL). Add MP-TsOH resin (˜4 equiv., 1.46 mmol/mg, Argonaut) to filtrate followed by dichloroethane (1 mL). Shake for 2-4 hr. Drain and wash with MeOH (1 mL, 3 times). Add 2M NH₃/MeOH (1.5 mL), shake for 30 min. Drain into vials. Add 2M NH₃/MeOH (2 mL) and shake for 10 min. and drain. Remove the solvent in to give the products in Table 11.

TABLE 11

| EX | | Mass |
|---|---|---|
| No. | COMPOUND | Spec |
| 106 | 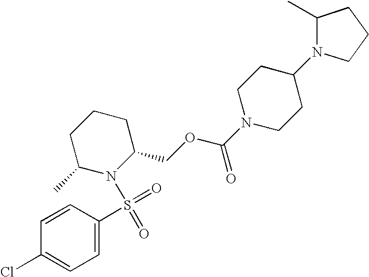 | 498 |
| 107 | 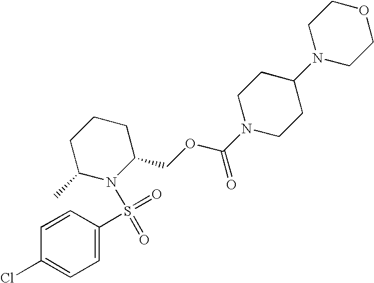 | 500 |
| 108 | 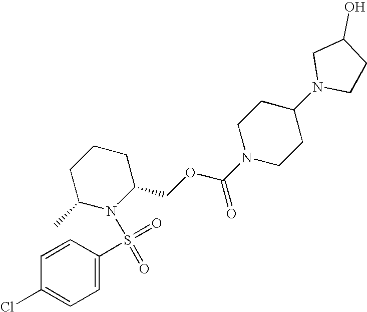 | 500 |
| 109 | 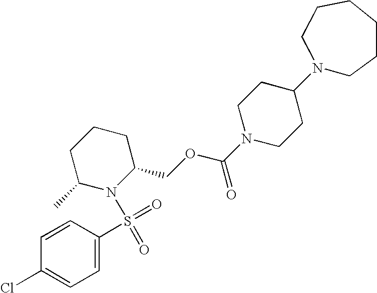 | 512 |
| 110 | 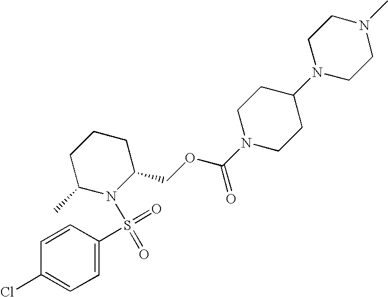 | 513 |
| 111 | 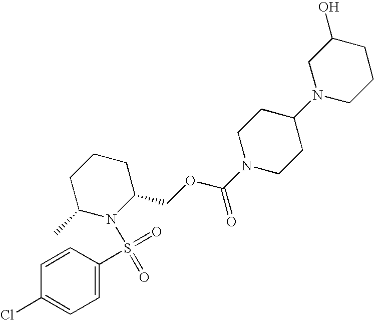 | 514 |
| 112 | 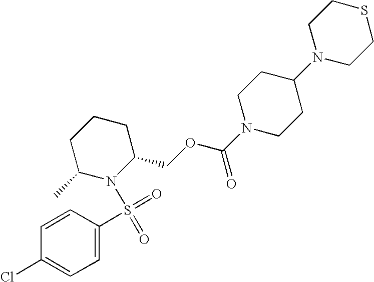 | 516 |
| 113 | 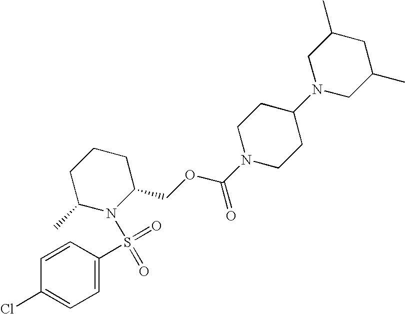 | 526 |
| 114 | 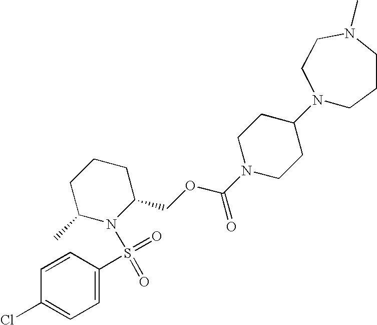 | 527 |
| 115 | 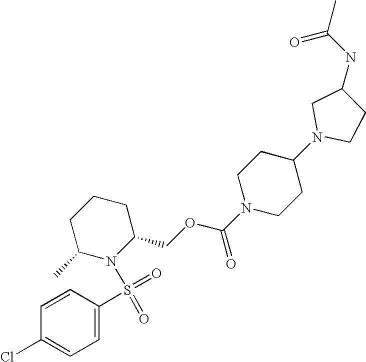 | 541 |
| 116 | 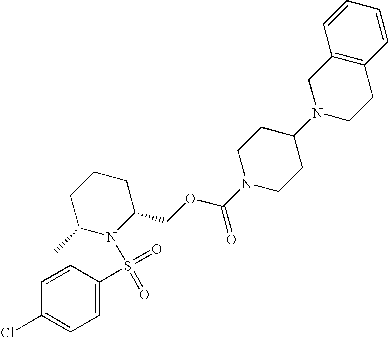 | 546 |
| 117 | 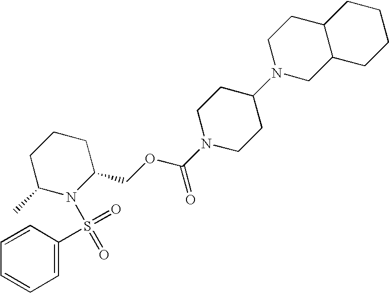 | 552 |
| 118 | 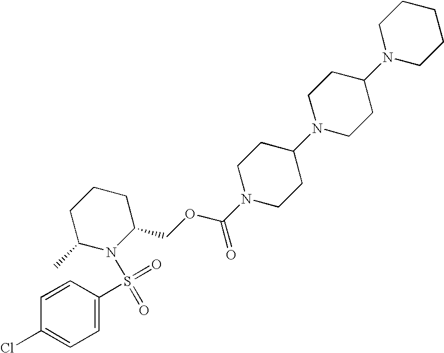 | 581 |
| 119 | 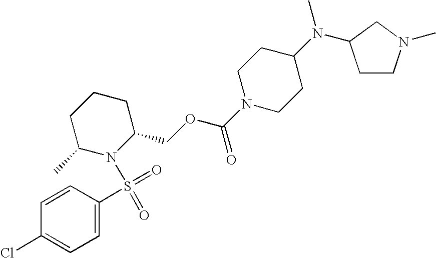 | 527 |
| 120 | 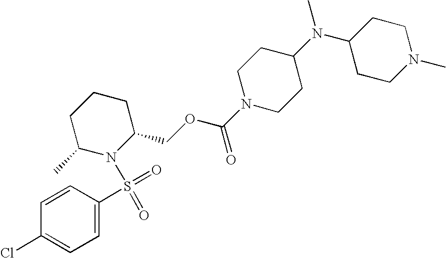 | 541 |
| 121 | 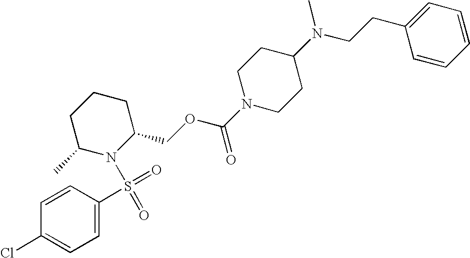 | 548 |
| 122 | 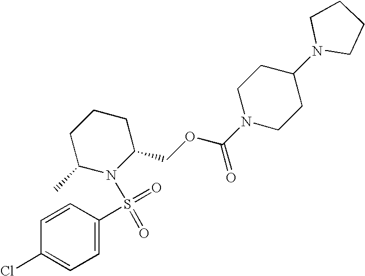 | 484 |
| 123 | 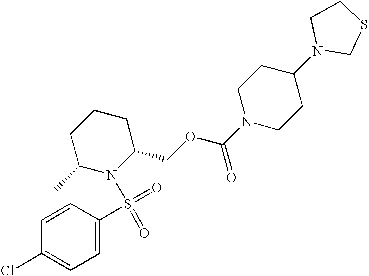 | 502 |
| 124 | 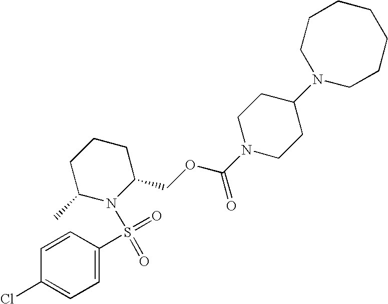 | 526 |
| 125 | 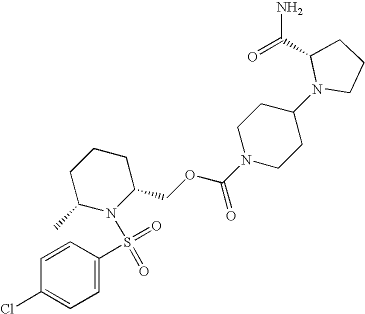 | 527 |
| 126 | 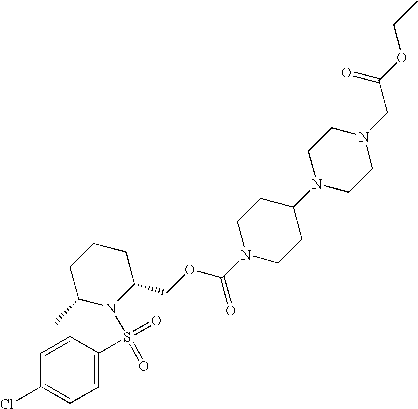 | 585 |
| 127 | 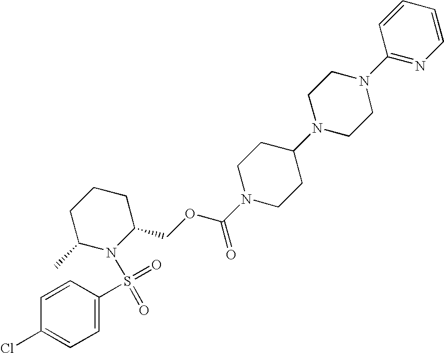 | 576 |
| 128 | 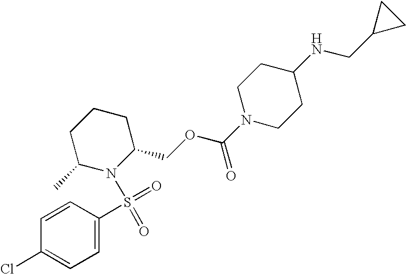 | 484 |
| 129 | 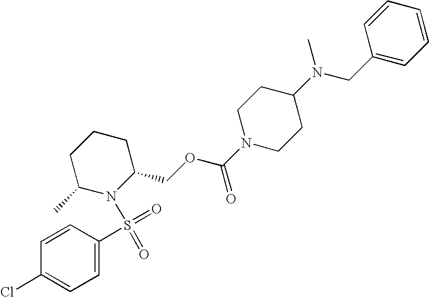 | 534 |
| 130 | 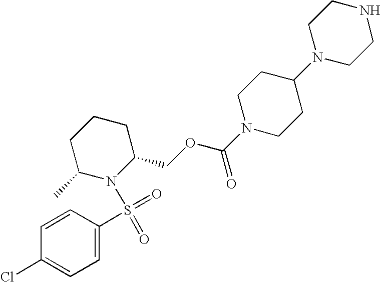 | 498 |
| 131 | 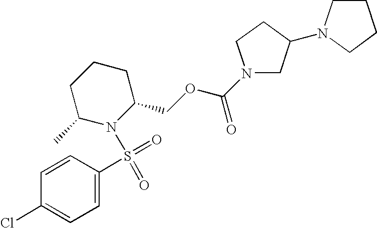 | 470 |
| 132 | 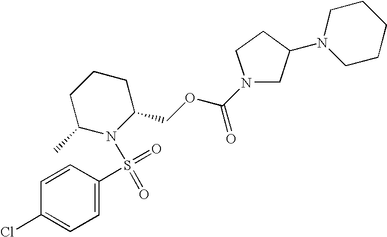 | 484 |
| 133 | 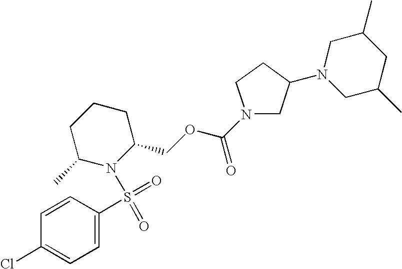 | 512 |
| 134 | 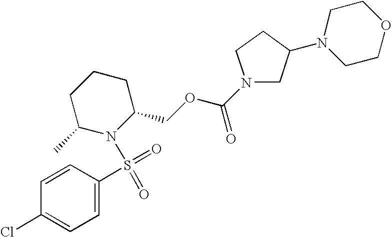 | 486 |
| 135 | 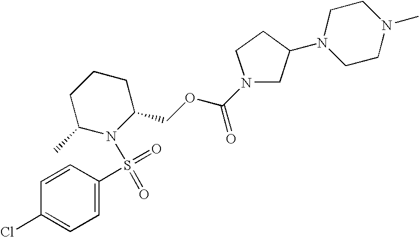 | 499 |
| 136 | 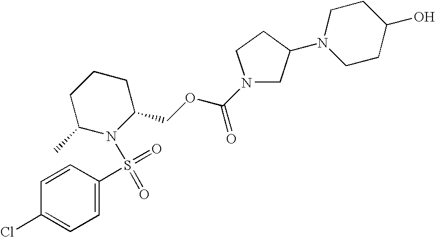 | 500 |
| 137 | 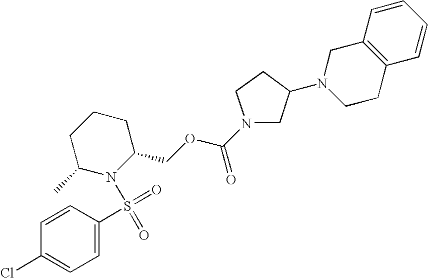 | 532 |
| 138 | 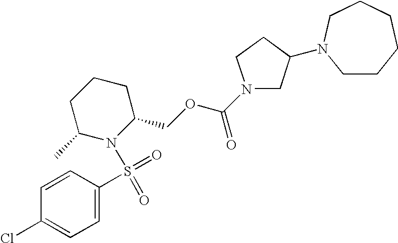 | 498 |
| 139 | 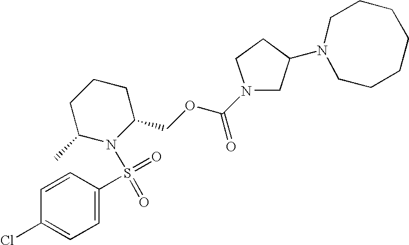 | 512 |
| 140 | 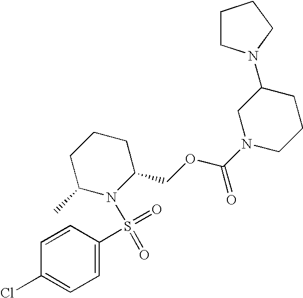 | 464 |
| 141 | 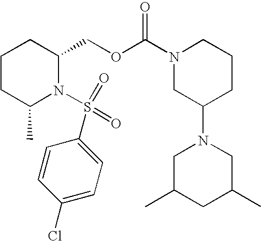 | 526 |
| 142 | 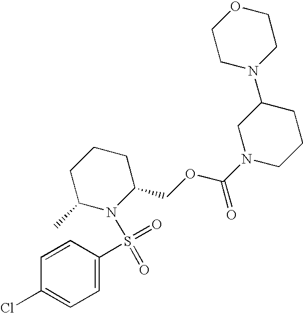 | 500 |
| 143 | 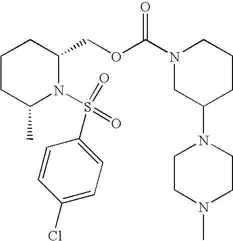 | 513 |
| 144 | 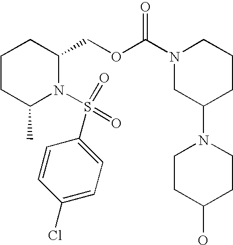 | 514 |
| 145 | 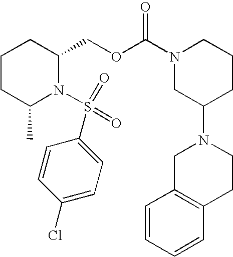 | 546 |
| 146 | 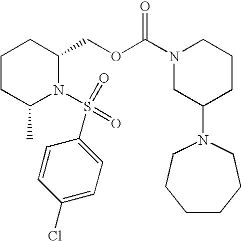 | 512 |
| 147 | 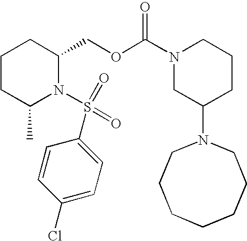 | 526 |
| 148 | 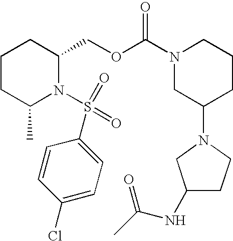 | 541 |
| 149 | 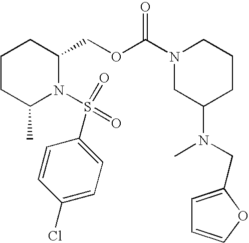 | 524 |
| 150 | 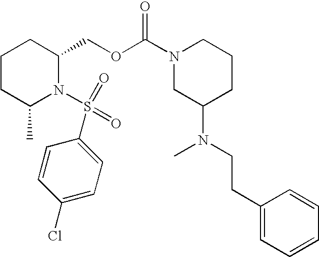 | 548 |
| 151 | 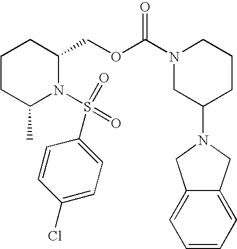 | 532 |
| 152 | 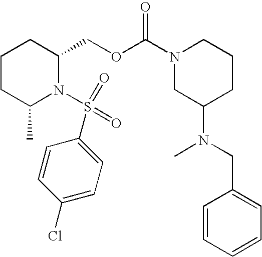 | 534 |
| 153 | 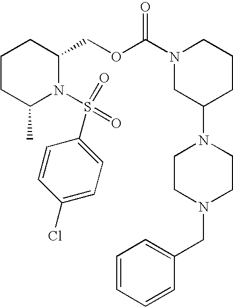 | 589 |
| 154 | 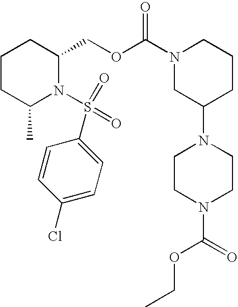 | 571 |
| 155 | 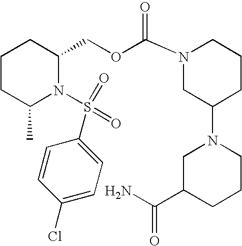 | 541 |
| 156 | 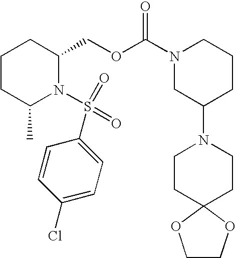 | 556 |
| 157 | 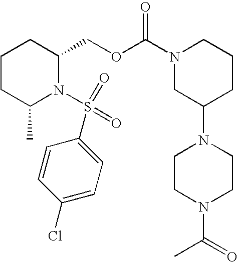 | 541 |
| 158 | 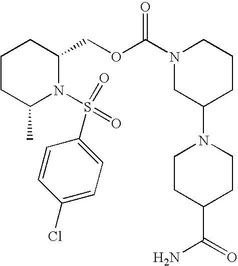 | 541 |

### Example 159

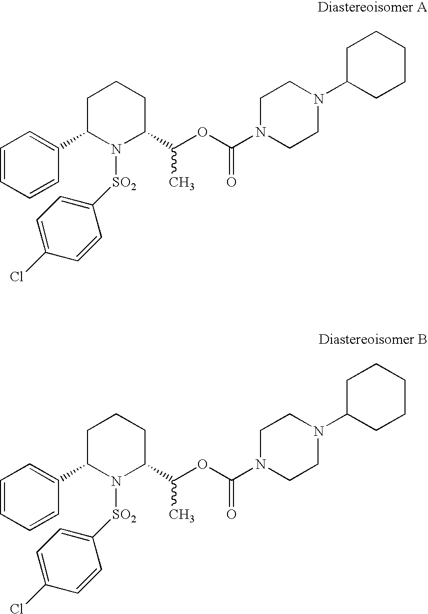

Step 1

To a solution of the 1-(4-chloro-benzenesulfonyl)-6-phenyl-piperidin-2-yl-methanol prepared according to Example 53 Preparation C Step 1 (300 mg; 0.82 mmol) in DCM (8 ml) was added Dess-Martin periodinane (850 mg; 2.0 mmol) followed by sodium bicarbonate (100 mg) and two drops of water. The mixture was stirred overnight at room temperature, then quenched with Et₂O (20 mL), saturated NaHCO₃ and sodium thiosulfite (2.0 g) for 20 minutes. The reaction was extracted with Et₂O, dried over Na₂SO₄ and concentrated to provide 232 mg (78%) of 1-(4-chloro-benzenesulfonyl)-6-phenyl-piperidine-2-carbaldehyde as an oil.

Step 2

To a solution of the product of step 1 (232 mg; 0.64 mmol) in THF (6 mL) at 0° C. was added methyl magnesium bromide solution 3 N in Et₂O (0.27 mL; 0.83 mmol) and the reaction was allowed to warm to room temperature for 1 h. The mixture was poured into saturated ammonium chloride, extracted with DCM, and dried over Na₂SO₄. After concentration of the solvents, the residue was purified by chromatography over silica gel (eluting Hexanes/EtOAc 8:2) to give 240 mg (100%) of 1-[1-(4-chloro-benzenesulfonyl)-6-phenyl-piperidin-2-yl]-ethanol as a ca 4.5:1 mixture of diastereoisomers.

Step 3

The product of Step 2 was converted to the title compounds according to Step 4 of Example 1, using N-cyclohexylpiperazine at the last stage as the amine. The diastereoisomers were separated at the last stage by chromatography on silica gel (eluting Hexanes/EtOAc 8:2) to provide, in order of elution:

(i) Diastereoisomer A: ¹H-NMR (300 MHz, CDCl₃) δ 7.86 (d, J=6.0 Hz, 2H), 7.60 (d, J=6.0 Hz, 2H), 7.53 (d, J=6.0 Hz, 2H), 7.30-7.45 (m, 2H), 7.20-7.30 (m, 1H), 5.25 (d, J=4.5 Hz, 2H), 4.35-4.50 (m, 1H), 3.90-4.00 (m, 1H), 3.20-3.50 (m, 4H), 2.15-2.60 (m, 5H), 1.70-2.05 (m, 5H), 1.50-1.65 (m, 2H), 1.00-1.45 (m, 9H), 0.99 (d, J=4.5 Hz, 2H); HRMS (MH⁺) 574.2500.

(ii) Diastereoisomer B: ¹H-NMR (300 MHz, CDCl₃) δ 7.84 (d, J=6.0 Hz, 2H), 7.45-7.60 (m, 4H), 7.25-7.40 (m, 3H), 5.23 (m, 1H), 4.30-4.45 (m, 1H), 4.05-4.20 (m, 1H), 3.30-3.70 (m, 4H), 2.20-2.70 (m, 5H), 1.75-2.00 (m, 5H), 1.05-1.70 (m, 14H); HRMS (MH⁺) 574.2512.

Some compounds prepared are shown below:

TABLE 12

| | | Retention | |
|---|---|---|---|
| | | Time | Observed |
| Compound No. | Structure | (minutes) | Mass |
| 159-A | 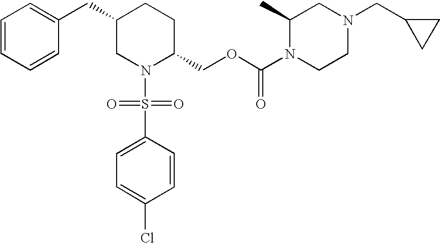 | 5.10 | 546.1 |
| 159-B | 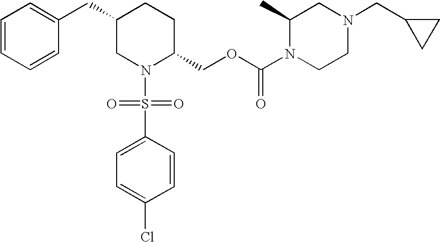 | 5.10 | 546.1 |

Preparations P-1 to P-4 describe preparation of intermediates used in several procedures.

Preparation P-1: Preparation of 4-[1-(4,4-ethylenedioxypiperidino)]piperidine:

Step 1:

A solution of 1-tert-butoxycarbonyl-4-piperidone (3.98 g, 20 mmol), 4-piperidoneethyleneketal (3.15 g, 22 mmol), sodium triacetoxyborohydride (4.66 g, 22 mmol), sodium sulfate (15 g) and acetic acid (300 μL) in DCE (15 mL) was stirred 2 days at RT. The solution was quenched with an excess of MeOH for 15 min then treated with diluted NaOH and extracted with DCM and AcOEt. The combined organic layers were dried over Na2SO4 and concentrated, and the crude was purified by flash-chromatography over silica gel (eluting DCM/AcOEt 7:3 to 1:1) to afford 4.72 g (72%) of 1-tert-butoxycarbonyl-4-[1-(4,4-ethylenedioxypiperidino)]piperidine.

Step 2:

To 1-tert-butoxycarbonyl-4-[1-(4,4-ethylenedioxy)piperidino]piperidine (200 mg, 061 mmol) in DCM (10 mL) was added TFA (1.5 mL), and the reaction was stirred 1 h 30. The reaction was treated with 1 N NaOH until pH>12 and extracted with DCM and AcOEt. The combined organic layers were dried over Na2SO4 and concentrated to provide 100 mg (75%) of 4-[1-(4,4-ethylenedioxypiperidino)]piperidine.

Preparation P-2: Preparation of 4-[1-(4-methoxyiminopiperidino)]piperidine:

Step 1:

To a solution of 4-piperidonemethoxime (150 mg, 1.17 mmol) in DCE (5 mL) was added 1-tert-butoxycarbonyl-4-piperidone (350 mg, 1.75 mmol) and the reaction was stirred 1 h at RT. Sodium triacetoxyborohydride (500 mg, 2.34 mmol) was added, followed by AcOH (20 μl), and the reaction was stirred 2 days at RT. The solution was quenched with an excess of MeOH for 15 min then treated with 5% NaHCO3 and extracted with DCM and AcOEt. The combined organic layers were dried over Na₂SO₄ and concentrated to provide 500 mg of crude 1-tert-butoxycarbonyl-4-[1-(4-methoxyiminopiperidino)]piperidine.

Step 2:

A solution of 1-tert-butoxycarbonyl-4-[1-(4-methoxyiminopiperidino)]piperidine (50 mg, 0.16 mmol) in DCM (2 mL) was treated with TFA (0.2 mL) and stirred at RT for 30 min. The reaction was concentrated, diluted with 1 N NaOH, and extracted with DCM and AcOEt. The combined organic layers were dried over Na2SO4 and concentrated to provide 50 mg (100%) of crude 4-[1-(4-methoxyiminopiperidino)]piperidine that could be used without purification in the next step.

Preparation P-3: Preparation of cis-3-methyl-4-(1-piperidino)piperidine:

Step 1:

To a solution of 1-benzyl-3-methylpiperidone (5.0 g, 24.6 mmol) in DCE was added piperidine (2.6 ml, 27.06 mmol) followed by Ti(OiPr)4 (8.8 ml, 29.52 mmol). The reaction was stirred at RT for 8 h, NaBH₃(CN) was added slowly and the mixture was then stirred 2 days at RT. The solution was quenched with an excess of MeOH for 15 min, treated with diluted NaOH, extracted with DCM and AcOEt, and the combined organic layers were dried over Na2SO4 and concentrated. Purification of a sample by flash-chromatography over silica gel (eluting hexanes/AcOEt 9:1 to 1:1) afforded 1.7 g of cis-1-benzyl-3-methyl4-(1-piperidino)piperidine.

Step 2:

A solution of cis-1-benzyl-3-methyl4-(1-piperidino)piperidine (1.7 g, 6.2 mmol), ammonium formate (6.3 g, 100 mmol) and palladium hydroxide on charcoal (1 g, 7.1 mmol) in MeOH (20 mL) was heated at reflux for 4 h. The final solution was filtered over Celite, rinsing with MeOH then concentrated. The residue was diluted with saturated NaHCO3, extracted with DCM and AcOEt, and combined organic layers were dried over Na2SO4 and concentrated to give 580 mg (52%) of cis-3-methyl-4-(1-piperidino)piperidine.

Preparation P-4: Preparation of 2′-Methyl-[1,4′]bipiperidine:

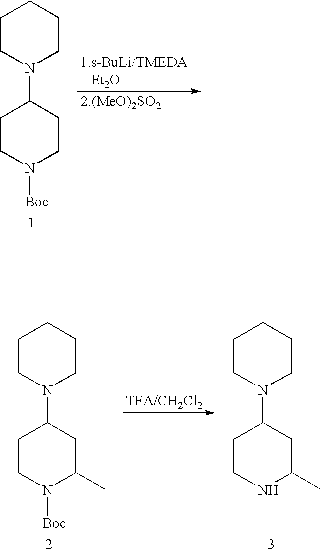

Compound 2: To a solution of 1′-tert-Butoxycarbonyl-[1,4′]-Bipiperidine 1 (5.1 g, 19.0 mmol), TMEDA (19 ml) in dry Et₂O (40 ml) at −78° C. is slowly added a solution of sec-butyllithium (19.0 ml, 24.7 mmol, 1.3 M in cyclohexanes) over a period of 30 min. The mixture is stirred at −78° C. for 3 hr, and then is treated with a solution of Dimethylsulfate (3.6 g, 28.5 mmol) in Et₂O (5 ml). The cooling bath is removed and the reaction mixture is stirred at ambient temperature for 16 hr. After cooling to 0° C., the reaction mixture is quenched with water, extracted with Et₂O (5×100 ml), and the combined ether layers is dried over K₂CO₃. The solvent is removed in vacuo and the residue is purified on silica gel chromatography (eluting 40% ethyl acetate in hexane) to give 2.51 g of 1′-tert-Butoxycarbonyl-2′-methyl-[1,4′]-Bipiperidine, 2.

Compound 3: To a stirring solution of compound 2 (1.5 g, 5.3 mmol) in DCM (10 ml) is added TFA, and the mixture is stirred at room temperature for 2 hr. After removing the volatiles, the residue is diluted with DCM, basified with 30% NH₄OH to pH 8 and the layer are separated. The organic phase is dried over MgSO₄ and concentrated to give 730 mg of 2′-Methyl-[1,4′]bipiperidinyl.

Specific examples are shown below:

### Example 160

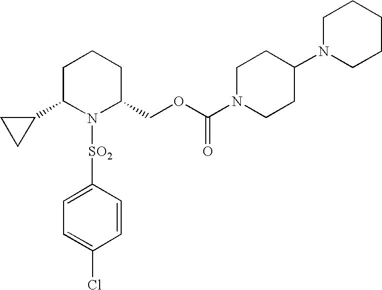

Step 1:

a) To a solution of 2-hydroxymethyl-6-(methoxycarbonyl)pyridine (44.5 g, 0.266 mol) in DCE (500 mL) was added triethylamine (44 mL, 0.31 mol) followed by TBSCl (44 g, 0.29 mol) and the reaction was heated at 70° C. for 4 h, then concentrated. The residue was directly purified by flash chromatography over silica gel (eluting hexane to hexane/AcOEt 1:1) to give 68.8 g (92%) of O-protected pyridine ester.

b) A solution of O-protected pyridine ester (68 g, 0.241 mmol) and platinum(IV) oxide (6 g, 0.026 mol) in MeOH (500 mL) and AcOH (50 ml) was hydrogenated 2 h at 40 psi. The final solution was filtered over Celite, rinsing with MeOH then concentrated. The residue was diluted with 1 N NaOH, extracted with DCM and AcOEt, and combined organic layers were dried over Na2SO4 and concentrated to provide 66 g (97%) of O-protected piperidine ester.

Step 2:

To a solution of O-protected piperidine ester (63 g, 0.22 mol) in DCE (500 mL) was added triethylamine (100 mL, 0.66 mol) then, slowly, 4-chlorobenzenesulfonyl chloride (93 g, 0.44 mol) and the reaction was heated at 40° C. overnight. The final mixture was concentrated and directly purified by flash chromatography over silica gel (eluting hexane to hexane/AcOEt 9:1) to afford 89 g (88%) of O-protected sulfonamide ester.

Step 3:

a) To a solution of O-protected sulfonamide ester (20.0 g, 43.3 mmol) in DCM (200 mL) at −78° C. was slowly added DIBAH 1 N in THF (45 ml, 45 mmol) and the reaction was stirred 1 h at this temperature. The reaction was then quenched with saturated sodium tartrate in water, warmed to room temperature, and diluted with DCM. Celite was added, the mixture was stirred 30 min and filtered. The solution was extracted with DCM and AcOEt and combined organic layers were dried over Na₂SO₄ and concentrated. The residue was purified by flash chromatography over silica gel (eluting hexane to hexane/AcOEt 1:1) to afford 15 g (80%) of O-protected sulfonamide aldehyde.

b) To a suspension of methyltriphenylphosphonium bromide (2.6 g, 7.2 mmol) in THF (25 mL) at −78° C. was added n-BuLi 2.5 N in hexanes (2.7 ml, 6.9 mmol). The solution was warmed to −20° C. for 30 min then treated with O-protected sulfonamide aldehyde (2.6 g, 6.0 mmol) dissolved in THF (25 mL). The reaction was allowed to warm to room temperature for 1 h then concentrated. The residue was taken up in saturated NaHCO3, extracted with DCM and AcOEt and combined organic layers were dried over Na₂SO₄ and concentrated. The residue was purified by flash chromatography over silica gel (eluting hexane to hexane/AcOEt 8:2) to give 2.1 g (85%) of O-protected sulfonamide alkene.

Step 4

a) To diethylzinc 1 N in hexanes (48.4 ml, 48.4 mmol) at 0° C. was added DCM (20 mL) followed by TFA (3.7 ml, 48.4 mmol) and the solution was stirred 5 min at this temperature. Diiodomethane (3.9 ml, 48.4 mmol) was then added followed 5 min later, by O-protected sulfonamide alkene (5.2 g, 12.1 mmol) in DCM (40 mL). The reaction was allowed to warm to room temperature for 2 h, diluted with water and extracted with DCM and AcOEt. Combined organic layers were dried over Na2SO4 and concentrated to give 5.7 g (100%) of O-protected cyclopropyl sulfonamide.

b) O-protected cyclopropyl sulfonamide (5.4 g, 12.1 mmol) was treated with TBAF following the conditions described in Example 1 Step 3-b to afford, after flash chromatography over silica gel (eluting hexane/AcOEt 9:1 to hexane/AcOEt 4:6), 4.0 g (100%) of cyclopropyl sulfonamide alcohol.

Optional Step 4-R: Optional Resolution of Cyclopropyl Sulfonamide Alcohol:

Cyclopropyl sulfonamide alcohol (0.75 g) was resolved by HPLC on Chiracel OJ column (eluting hexane/isopropanol 95:5) to afford, in order of elution, 276 mg of enantiomer A and 296 mg of enantiomer B, both as oils.

Step 5

The product of step 4 was converted to the title compound according to conditions similar to the ones described in Step 4 of Example 1, using 4-(1-piperidino)piperidine at the last stage as the amine. ¹H-NMR (300 MHz, CDCl₃) δ 7.74 (d, J=8.4 Hz, 2H), 7.45 (d, J=8.4 Hz, 2H), 4.10-4.40 (m, 5H), 3.24 (m, 1H), 2.40-2.90 (m, 7H), 1.05-1.90 (m, 17H), 0.70 (m, 1H), 0.59 (m, 2H), 0.25 (m, 1H); HRMS (MH⁺) 524.2356.

Following procedures similar to those in Example 160, the following compounds were prepared:

TABLE 13

| | | Retention | |
|---|---|---|---|
| Compound | | Time | Observed |
| No. | Structure | (minutes) | Mass |
| 160-A | 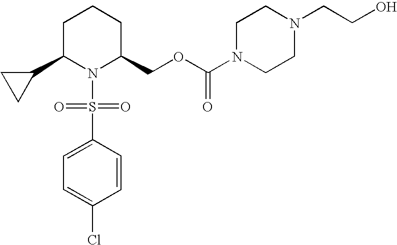 | 4.60 | 486.1 |
| 160-B | 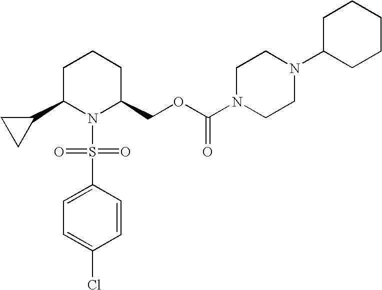 | 5.00 | 524.1 |
| 160-C | 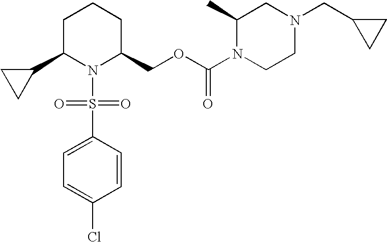 | 5.30 | 510.1 |
| 160-D | 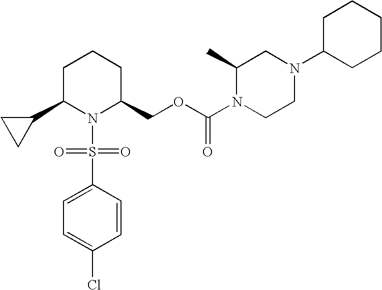 | 5.40 | 538.1 |
| 160-E | 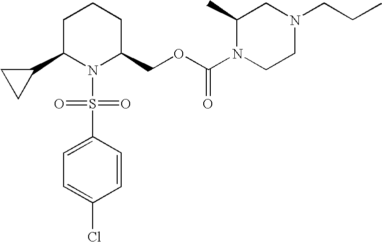 | 5.30 | 498.1 |
| 160-F | 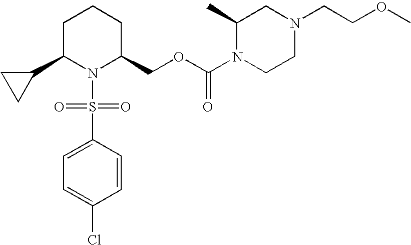 | 5.40 | 514.1 |
| 160-G | 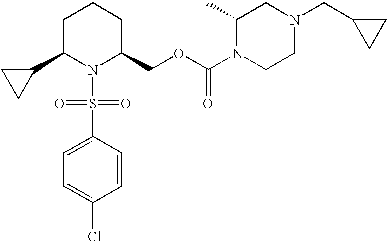 | 5.30 | 510.1 |
| 160-H | 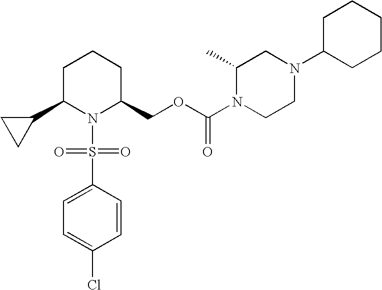 | 5.50 | 538.1 |
| 160-I | 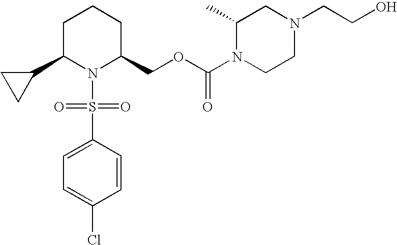 | 5.00 | 500.1 |
| 160-J | 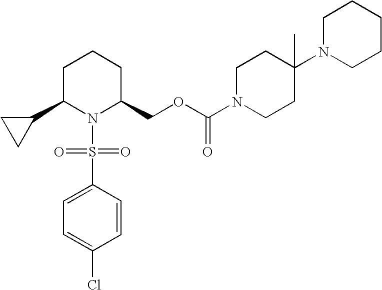 | 5.70 | 538.1 |
| 160-K | 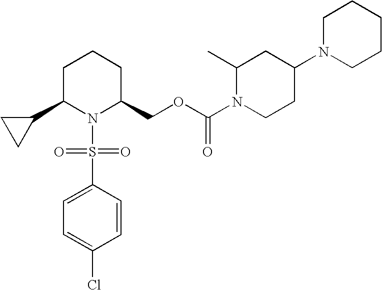 | 5.90 | 538.1 |
| 160-L | 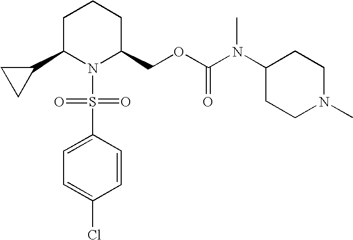 | 4.50 | 484.1 |
| 160-M | 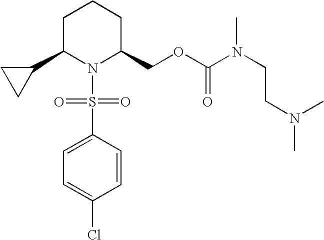 | 4.70 | 458.3 |
| 160-N | 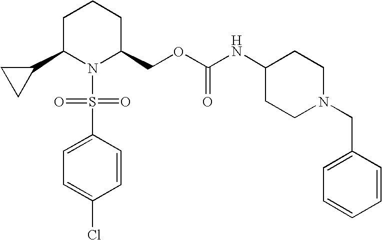 | 4.80 | 546.3 |
| 160-O | 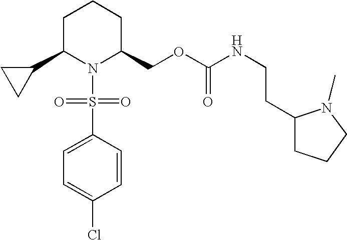 | 4.60 | 484.3 |
| 160-P | 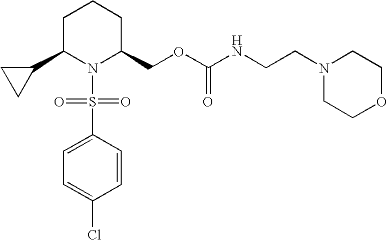 | 4.30 | 486.3 |
| 160-Q | 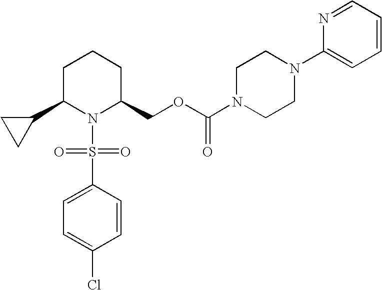 | 4.80 | 519.3 |
| 160-R | 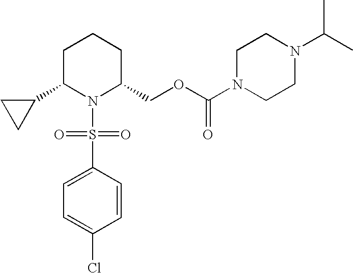 | 5.00 | 484.1 |
| 160-S | 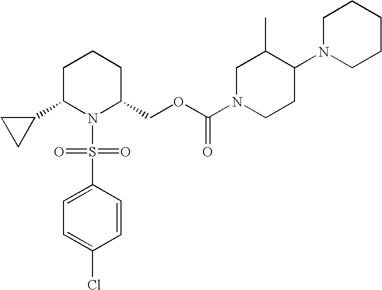 | 5.30 | 538.1 |
| 160-T | 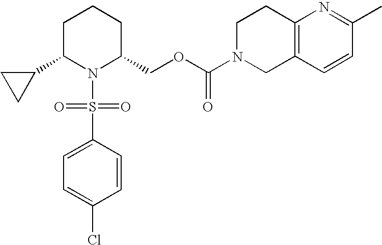 | 5.10 | 504.1 |
| 160-U | 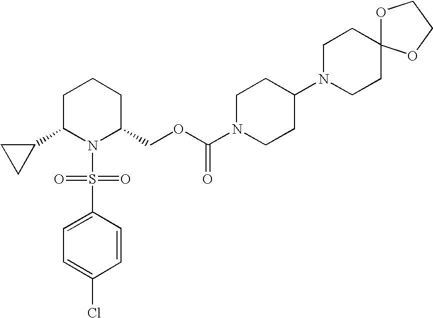 | 5.60 | 582.1 |
| 160-V | 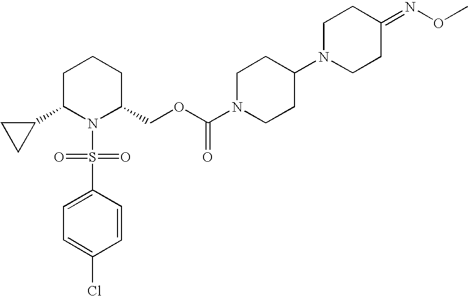 | 4.90 | 567.1 |
| 160-W | 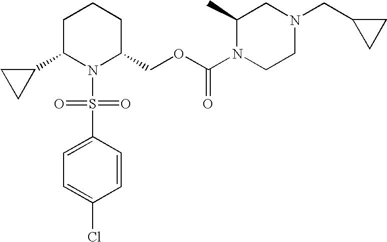 | 4.70 | 510.3 |
| 160-X | 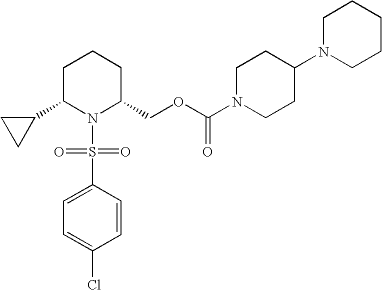 | 5.10 | 526.1 |
| 160-Y | 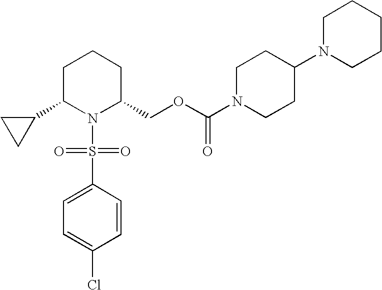 | 5.00 | 526.1 |
| 160-Z | 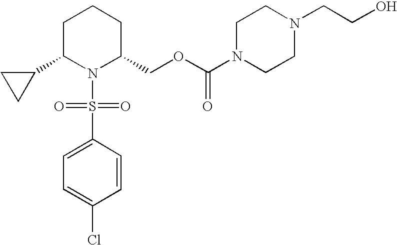 | 4.50 | 486.1 |
| 160-AA | 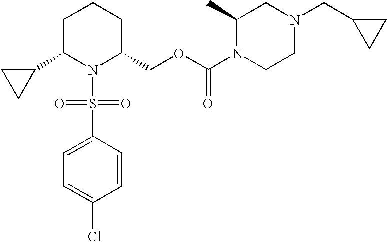 | 4.60 | 510.3 |

### Example 161

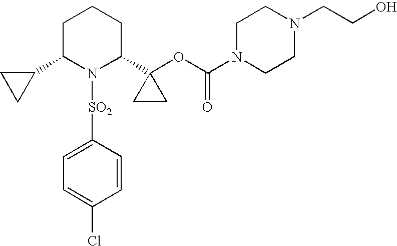

Step 1:

a) To a solution of cyclopropyl sulfonamide alcohol product of Example 160 Step 4-b (4.8 g, 14.5 mmol) in AcOEt (25 mL), acetonitrile (25 mL) and water (50 mL) was added sodium periodate (9.3 g, 43.5 mmol) followed by RuCl3.nH2O (100 mg). The reaction was stirred at RT for 2 hr, filtered over Celite, and extracted with AcOEt. Combined organic layers were dried over Na2SO4 and concentrated to provide 4.55 g (90%) of cyclopropyl sulfonamide acid.

b) A solution of cyclopropyl sulfonamide acid (4.55 g, 13.2 mmol) in MeOH (100 mL) was treated with thionyl chloride (2 ml, 26.5 mmol) at RT slowly then the solution was heated to reflux for 2 hr. The reaction was concentrated, diluted with saturated NaHCO3, extracted with DCM and AcOEt and combined organic layers were dried over Na2SO4 and concentrated. The residue was purified by flash chromatography over silica gel (eluting hexane to hexane/AcOEt 1:1) to afford 3.0 g (64%) of cyclopropyl sulfonamide ester.

Step 2:

To a solution of cyclopropyl sulfonamide ester (600 mg, 1.7 mmol) in THF (10 mL) was added Ti(OiPr)4 (0.1 ml, 0.34 mmol), then the reaction was cooled to 10° C. and slowly treated with EtMgBr 3 N in ether (1.7 ml, 5.1 mmol) over 30-40 min. The mixture was stirred another 30 min at 10° C., then treated with saturated NH4Cl at this temperature, and extracted with DCM and AcOEt. Combined organic layers were dried over Na2SO4, concentrated, and the residue was purified by flash chromatography over silica gel (eluting hexane to hexane/AcOEt 1:1) to yield 370 mg (61%) of cyclopropyl sulfonamide cyclopropylalcohol.

Step 3:

The product of step 2 was converted to the title compound according to conditions similar to the ones described in Step 4 of Example 1, using 1-(2-hydroxyethyl)piperazine at the last stage as the amine. ¹H-NMR (300 MHz, CDCl₃) δ 7.69 (d, J=8.8 Hz, 2H), 7.42 (d, J=8.8 Hz, 2H), 4.56 (d, J=6.6 Hz, 1H), 3.30-3.75 (m, 6 H), 3.01 (m, 1H), 2.30-2.65 (m, 6H), 1.40-1.70 (m, 4H), 0.95-1.25 (m, 8H), 0.73 (m, 1H), 0.58 (m, 2H), 0.23 (m, 1H); HRMS (MH⁺) 512.1992.

Following procedures similar to those in Example 161 the following compounds were prepared:

TABLE 14

| Compound | | Retention Time | Observed |
|---|---|---|---|
| No. | Structure | (minutes) | Mass |
| 161-A | 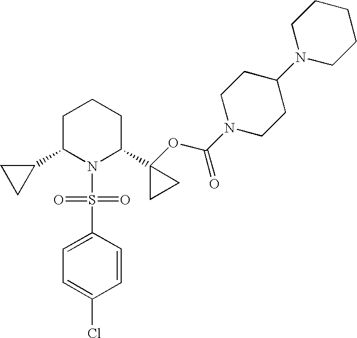 | 4.60 | 550.3 |
| 161-B | 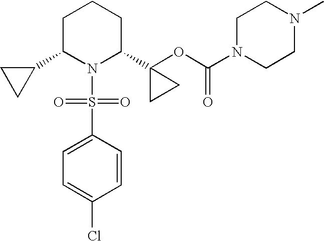 | 4.40 | 482.3 |
| 161-C | 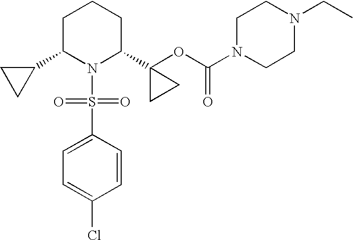 | 4.40 | 496.3 |
| 161-D | 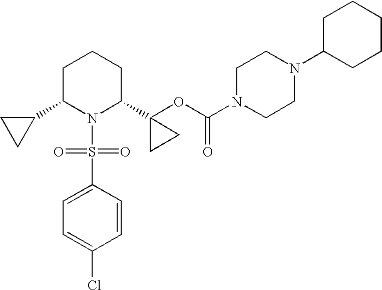 | 4.90 | 550.3 |
| 161-E | 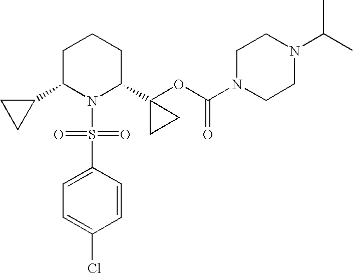 | 4.60 | 510.3 |
| 161-F | 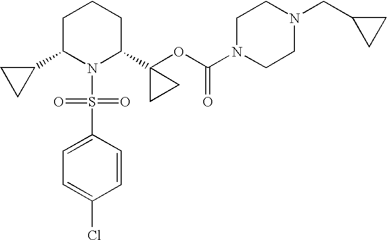 | 4.90 | 522.1 |
| 161-G | 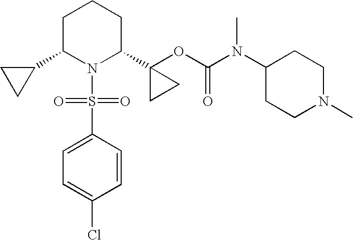 | 4.80 | 510.3 |

### Example 162

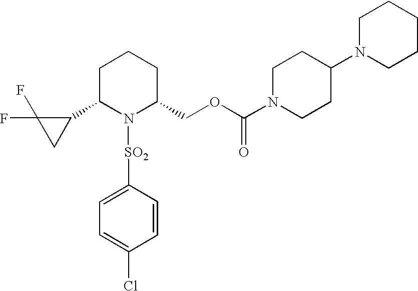

Step 1:

To a solution of O-protected sulfonamide alkene product of Example 160 Step 3-b (480 mg, 1.12 mmol) and sodium fluoride (1 mg) in toluene (0.2 mL) at 100° C. was added FSO2CF2COOTMS (700 mg, 2.8 mmol) over 1 h and the reaction was stirred an additional 2 h at this temperature. The final mixture was concentrated and purified over silica gel (eluting hexane/AcOEt 9:1) to afford 338 mg of starting material and 65 mg (41% based on recovery) of O-protected difluorocyclopropyl sulfonamide.

Step 2:

The product of step 1 was converted to the title compound according to conditions similar to the ones described in Example 1 Step 3-b and Step 4, using 4-(1-piperidino)piperidine at the last stage as the amine. ¹H-NMR (300 MHz, CDCl₃) δ 7.78 (d, J=8.4 Hz, 2H), 7.46 (d, J=8.4 Hz, 2H), 3.90-4.35 (m, 6H), 3.47 (s, 1H), 2.60-2.80 (m, 2H), 2.35-2.60 (m, 5H), 1.70-2.05 (m, 5H), 1.20-1.70 (m, 12H), 1.06 (m, 1H); HRMS (MH⁺) 560.2153.

Following procedures similar to those in Example 162 the following compounds were prepared.

TABLE 15

| Compound | | Retention Time | Observed |
|---|---|---|---|
| No. | Structure | (minutes) | Mass |
| 162-A | 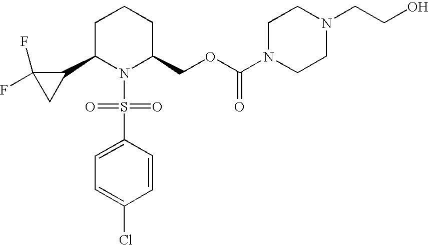 | 4.10 | 522.3 |
| 162-B | 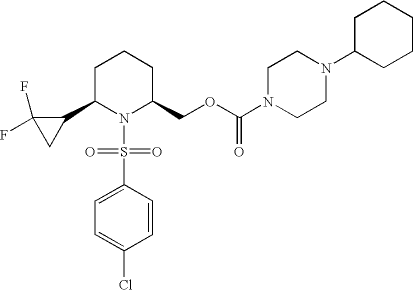 | 4.90 | 560.3 |

### Example 163

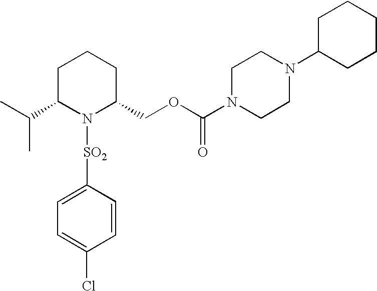

Step 1:

a) To a solution of O-protected pyridine ester product from Example 160 Step 1-a (10.0 g, 36 mmol) in THF (140 mL) at 0° C. was slowly added MeMgBr 3 N in ether (30 ml, 90 mmol), and the reaction was warmed to RT and stirred 1 h. The final mixture was poured into 1 N NaOH and DCM to which was added Celite, stirred, and filtered. The aqueous layer was extracted with DCM and AcOEt, combined organic layers were dried over Na2SO4 and concentrated, and the residue was purified by flash chromatography over silica gel (eluting hexane/AcOEt 8:2) to give 3.0 g (30%) of O-protected pyridine dimethylcarbinol.

b) To a solution of O-protected pyridine dimethylcarbinol (3.0 g, 10.6 mmol) in THF (50 mL) at −78° C. was added n-BuLi 2.5 N in hexanes (4.7 ml, 11.7 mmol) followed, 1 min later, by phenylthionochloroformate (2.76 g, 16.0 mmol). The reaction was stirred at −78° C. for 40 min, then allowed to warm to RT and stirred 4 h. The final mixture was treated with saturated NaHCO3, extracted with DCM and AcOEt and combined organic layers were dried over Na2SO4 and concentrated. Purification of the residue by flash chromatography over silica gel (eluting hexane to DCM) afforded 1.5 g of O-protected pyridine propene as well as 1.8 g of starting O-protected pyridine dimethylcarbinol.

Step 2:

a) A solution of O-protected pyridine propene (1.5 g, 5.7 mmol) and platinum(IV) oxide (258 mg) in MeOH (20 mL) and AcOH (4 ml) was hydrogenated 6 h at 40 psi. The final solution was filtered over Celite, rinsing with MeOH then concentrated. The residue was diluted with 1 N NaOH, extracted with DCM and AcOEt, and combined organic layers were dried over Na2SO4 and concentrated. The residue was quickly passed through a plug of silica gel (eluting hexanes/AcOEt 8:2) to provide 1.0 g (65%) of O-protected isopropyl piperidine.

b) A solution of O-protected isopropyl piperidine (0.82 g, 3.0 mmol), 4-chlorobenzenesulfonyl chloride (1.2 g, 6.0 mmol) and pyridine (10 mL) in DCE (10 mL) was heated at 60° C. overnight. The final mixture was concentrated and directly purified by flash chromatography over silica gel (eluting hexane to DCM) to afford 0.42 g (32%) of O-protected isopropyl sulfonamide.

Step 3:

The product of step 2 was converted to the title compound according to conditions similar to the ones described in Example 1 Step 3-b and Step 4, using 1-cyclohexylpiperazine at the last stage as the amine. ¹H-NMR (300 MHz, CDCl₃) δ 7.77 (d, J=8.8 Hz, 2H), 7.47 (d, J=8.8 Hz, 2H), 4.05-4.30 (m, 3H), 3.40-3.70 (m, 5H), 2.53 (br s, 4H), 2.27 (m, 1H), 1.35-2.00 (m, 10H), 0.95-1.35 (m, 10H), 0.91 (dm J=6.6 Hz, 3H); HRMS (MH⁺) 526.2501.

Following procedures similar to those in Example 163 the following compounds were prepared.

TABLE 16

| Compound | | Retention Time | Observed |
|---|---|---|---|
| No. | Structure | (minutes) | Mass |
| 163-A | 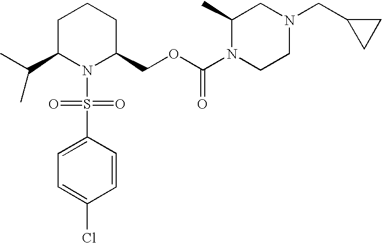 | 5.40 | 512.1 |
| 163-B | 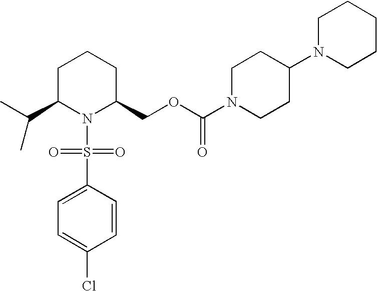 | 5.60 | 526.1 |
| 163-C | 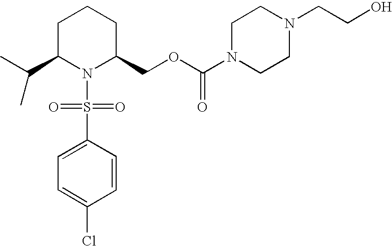 | 5.10 | 488.1 |

### Example 164

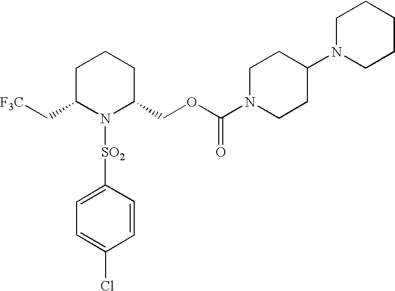

Step 1:

a) To a solution of O-protected pyridine ester product from Example 160 Step 1-a (45.75 g, 0.16 mol) in DCM (500 mL) at −40° C. was slowly added DIBAH 1 N in hexane (211 ml, 0.21 mmol) and the reaction was stirred 1 h at this temperature. The reaction was then quenched with an excess of acetone, then treated with sodium fluoride (25 g) solution in water (100 mL) for 30 min. The final mixture was filtered over Celite, extracted with DCM and AcOEt and combined organic layers were dried over Na2SO4 and concentrated. The residue was purified by flash chromatography over silica gel (eluting hexane/AcOEt 8:2) to afford 27.2 g (68%) of O-protected pyridine aldehyde.

b) To a solution of O-protected pyridine aldehyde (5.0 g, 19.9 mmol) and TBAF 1 N in THF (1.5 mL, 1.5 mmol) in THF (60 mL) at 0° C. was slowly trifluoromethyltrimethylsilane (3.4 mL, 20.9 mmol) and the reaction was allowed to warm to RT overnight. The reaction was diluted with water and DCM, extracted with DCM, dried over Na2SO4 and concentrated. The residue was purified by flash chromatography over silica gel (eluting hexane/AcOEt 8:2) to afford 1.5 g (24%) of O-protected pyridine trifluoroethyl alcohol.

Step 2:

a) To a solution of O-protected pyridine trifluoroethyl alcohol (1.8 g, 5.6 mmol) in THF (30 mL) at −78° C. was added n-BuLi 2.5 N in hexanes (2.5 ml, 6.2 mmol) followed, 1 min later, by phenylthionochloroformate (1.45 g, 8.4 mmol). The reaction was stirred at −78° C. for 40 min, then allowed to warm to RT and stirred an additional 1 h. The final mixture was then diluted with saturated NaHCO3, extracted with DCM and AcOEt and combined organic layers were dried over Na2SO4 and concentrated. Purification of the residue by flash chromatography over silica gel (eluting DCM/hexane 1:1) afforded 2.3 g (92%) of O-protected pyridine trifluoroethyl thionocarbonate.

b) To a solution of O-protected pyridine trifluoroethyl thionocarbonate (2.3 g, 5.0 mmol) in toluene (60 mL) was added tributyltin hydride (3.0 mL, 10.5 mmol) followed by 2,2′-azobisisobutyronitrile (265 mg, 1.6 mmol) and the reaction was heated under reflux for 5 h. After concentration of the solvent, the residue was purified by flash chromatography over silica gel (eluting hexane to DCM/hexane 1:1) to give 1.3 g (86%) of O-protected trifluoroethyl pyridine.

Step 3:

a) A solution of O-protected trifluoroethyl pyridine (1.3 g, 4.3 mmol) and platinum(IV) oxide (100 mg) in MeOH (50 mL) and AcOH (5 ml) was hydrogenated overnight at 50 psi. The final solution was filtered over Celite, rinsing with MeOH then concentrated. The residue was diluted with 1 N NaOH, extracted with DCM and AcOEt, and combined organic layers were dried over Na2SO4 and concentrated to provide 1.13 g (84%) of O-protected trifluoroethyl piperidine.

b) To a solution of O-protected trifluoroethyl piperidine (1.13 g, 3.6 mmol) in DCE (15 mL) was added triethylamine (0.6 mL, 4.3 mmol) then 4-chlorobenzenesulfonyl chloride (1.13 g, 5.4 mmol) and the reaction was heated at reflux overnight. The final mixture was concentrated and directly purified by flash chromatography over silica gel (eluting hexane to DCM) to afford 0.67 g (38%) of O-protected trifluoroethyl sulfonamide.

Step 4:

The product of step 3 was converted to the title compound according to conditions similar to the ones described in Example 1 Step 3-b and Step 4, using 4-(1-piperidino)piperidine at the last stage as the amine. ¹H-NMR (300 MHz, CDCl₃) δ 7.77 (d, J=8.3 Hz, 2H), 7.48 (d, J=8.3 Hz, 2H), 4.10-4.45 (m, 5H), 3.99 (m, 1H), 2.40-2.95 (m, 9H), 1.20-2.00 (m, 16H); HRMS (MH⁺) 566.2075.

Following procedures similar to those in Example 164, the following compounds were prepared.

TABLE 17

| Compound | | Retention Time | Observed |
|---|---|---|---|
| No. | Structure | (minutes) | Mass |
| 164-A | 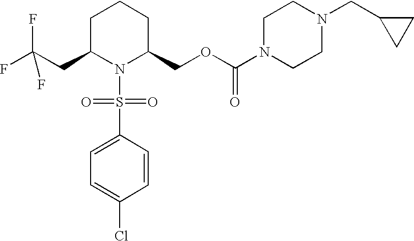 | 5.00 | 538.1 |
| 164-B | 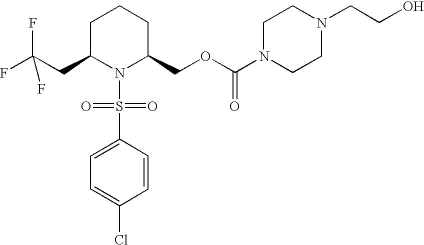 | 4.60 | 528.1 |
| 164-C | 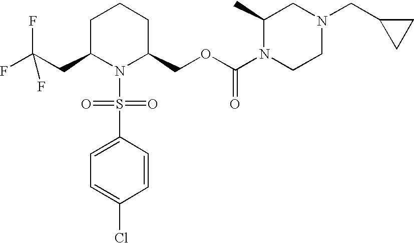 | 4.90 | 552.1 |

### Example 165

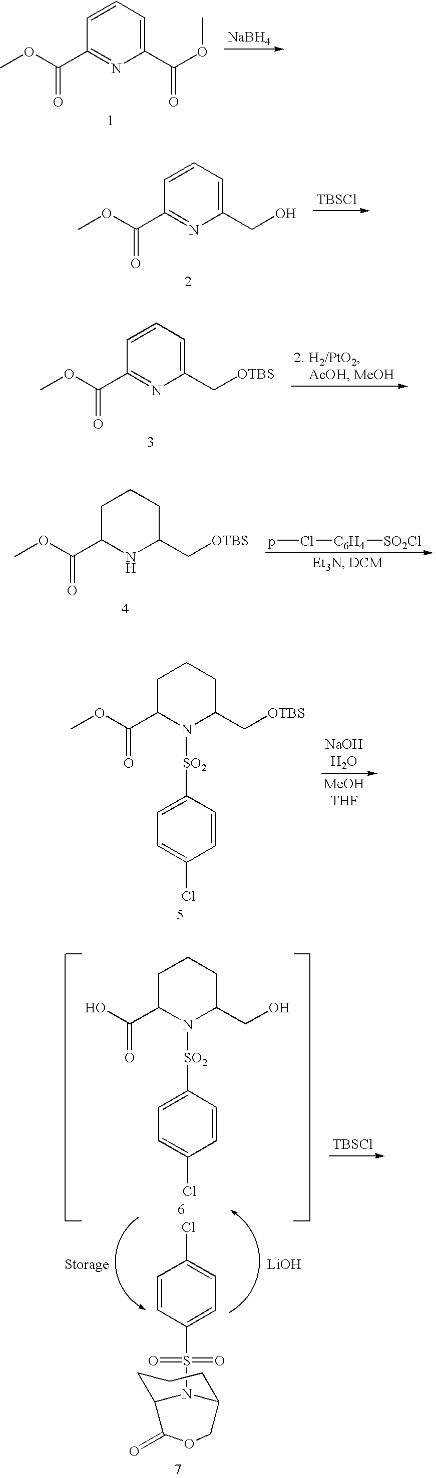

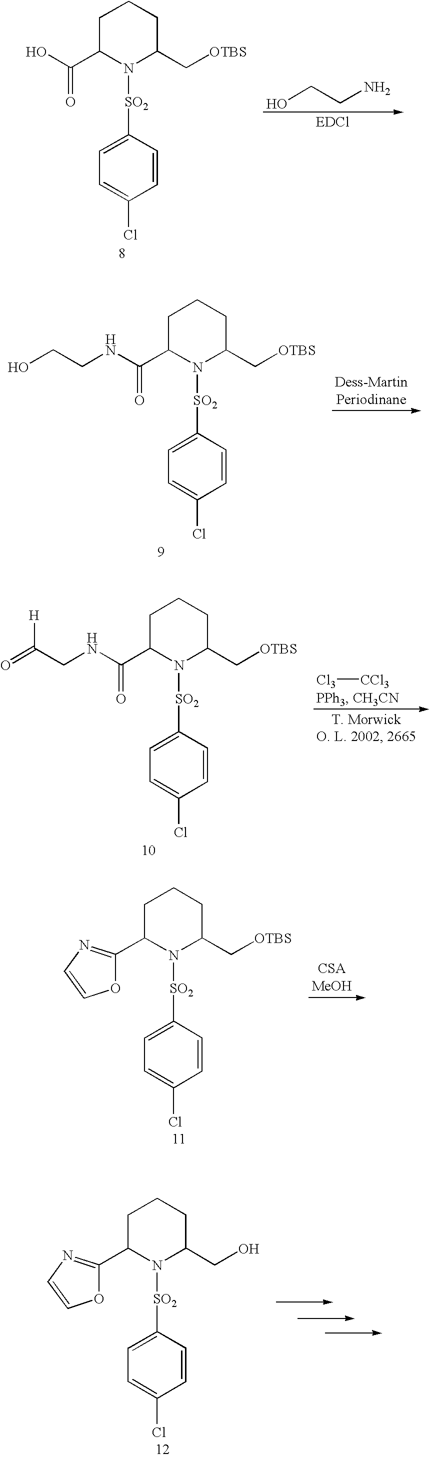

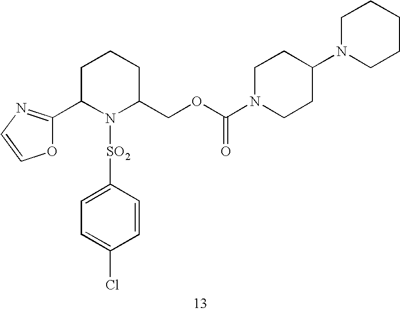

Step 1: Compound 2 is prepared as described in Example 88, Step 1.

Step 2: A mixture of 1.396 g (8.35 mmol) of Compound 2 and 1.137 g (19.71 mmol) of imidazole in 10 ml of DMF was treated with 1.210 g (9.18 mmol) of TBSCl. After overnight stirring, the mixture was diluted with DCM, washed with water, dried over sodium sulfate and concentrated. The product was purified by chromatography using 10% ethyl acetate in hexanes as solvent to furnish 1.65 g of Compound 3.

Step 3: Compound 3 (4.0 g) was hydrogenated at 50 psi using 200 mg of PtO₂ as catalyst and a mixture of 20 ml of methanol and 20 ml of acetic acid as solvent over a period of 12 h. The reaction vessel was flushed with nitrogen, catalyst was filtered out and volatiles were evaporated. The residue was re-dissolved in DCM, washed with sat. NaHCO₃, aqueous phase was re-extracted with DCM, combined organic phase was dried over sodium sulfate and concentrated to furnish 3.77 g of Compound 4.

Step 4: A mixture of 3.77 g (13.13 mmol) of Compound 4, 7.4 ml (52.6 mmol) of triethylamine and 5.54 g (26.26 mmol) of 4-chlorobenzenesulfonyl chloride in 60 ml of DCM was stirred over 7 days. The mixture was diluted with DCM, washed with water, dried over sodium sulfate and concentrated. The product was purified by chromatography using 5-15% of ethyl acetate in hexanes as solvent to furnish 4.99 g of Compound 5.

Step 5: A mixture of 150 mg of Compound 5, 5 ml of methanol, 5 ml of THF and 5.0 ml of 1 M aqueous NaOH was refluxed overnight. The mixture was cooled, DCM (100 ml) and 1 M HCl were added so that pH was adjusted to ˜3. Organic layer was separated, aqueous phase was extracted with DCM. Combined organic phase was dried over sodium sulfate and concentrated to furnish 90 mg of unstable Compound 6, which had a tendency to dehydrate on storage to provide Compound 7. In order to regenerate Compound 6 from Compound 7, the following procedure was used:

A mixture of 500 mg of Compound 7, 4.0 ml of THF, 0.7 ml of water and 72 mg of LiOH was vigorously stirred overnight. Reaction mixture was diluted with ethyl acetate and pH was adjusted to ˜3 with 1 M HCl. Organic layer was separated, aqueous phase was extracted with DCM. Combined organic phase was dried over sodium sulfate and concentrated to furnish 310 mg of unstable Compound 6.

Step 6: Stirred overnight a mixture of 310 mg (0.931 mmol) of freshly prepared Compound 6, 349 mg (2.33 mmol) of TBSCl, 272 mg (4 mmol) of imidazole and 5 ml of DMF. The mixture was diluted with DCM, partitioned with citric acid, aqueous phase was re-extracted with DCM. Combined organic phase was dried over sodium sulfate and concentrated. The product was purified by chromatography using 30% of ethyl acetate in hexanes as solvent to furnish 350 mg of Compound 8.

Step 7: To a mixture of 350 mg (0.783 mmol) of Compound 8, 95 mg (1.56 mmol) of ethanolamine in 5 ml of DMF was added 211 mg (1.56 mmol) of HOBt, 300 mg (1.56 mmol) of EDCl, and 0.218 ml (1.56 mmol) of triethylamine. The turbid mixture was stirred overnight, diluted with DCM, washed with water, dried over sodium sulfate and concentrated. The product was purified by chromatography using 40% of ethyl acetate in hexanes as solvent to furnish 138 mg of Compound 9.

Step 8. To a solution of 138 mg (0.2816 mmol) of Compound 9 in 2 ml of DCM was added 238 mg (0.563 mmol) of Dess-Martin periodinane. The mixture was stirred over a period of 1 h, diluted with DCM, washed with sat. NaHCO₃, dried over sodium sulfate and concentrated. The product was purified by chromatography using 40% of ethyl acetate in hexanes as solvent to furnish 110 mg of Compound 10.

Step 9. To a mixture of 80 mg (0.1638 mmol) of Compound 10 in 3 ml of acetonitrile was added 194 mg (0.82 mmol) of hexachloroethane, 0.23 ml (1.64 mmol) of triethylamine followed by 215 mg (0.82 mmol) of triphenylphosphine. (The latter reagent dissolved gradually, then a new precipitate forms after 10 min of stirring). The mixture was stirred overnight and Compound 11 (56 mg) was isolated by prep. TLC chromatography using 20% ethyl acetate in hexanes as solvent.

Step 10. A mixture of 56 mg (0.119 mmol) of Compound 11 in 1.5 ml of THF was treated with 0.24 ml (0.24 mmol) of 1M TBAF solution in THF. The reaction mixture was stirred for 1 h, poured into water, extracted with DCM, organic phase was dried over sodium sulfate and concentrated to furnish 50 mg of crude Compound 12, which was used without further purification.

Step 11. Compound 13 was prepared from Compound 12 using procedures similar to Example 1, Step 4(a) and 4(b), except that step 4(a) was modified so that a 2:1 mixture of THF and acetonitrile was used as solvent instead of DCM.

¹H NMR (CDCl₃, 400 MHz) δ 7.86 (2H, d, J=8.8 Hz), 7.63 (1H, s), 7.51 (2H, d, J=8.8 Hz), 7.09 (1H, s), 5.32 (1H, d, J=5.0 Hz), 4.25 (1H, m), 4.14 (1H, br), 3.73 (1H, t, J=9.0 Hz), 3.58 (1H, t, J=9.0 Hz), 2.70 (2H, m), 2.52-2.33 (6H, ser. m.), 2.0-1.2 (16H, ser. m.); MS (ES) m/e 552.1 (M+H)⁺.

### Example 166

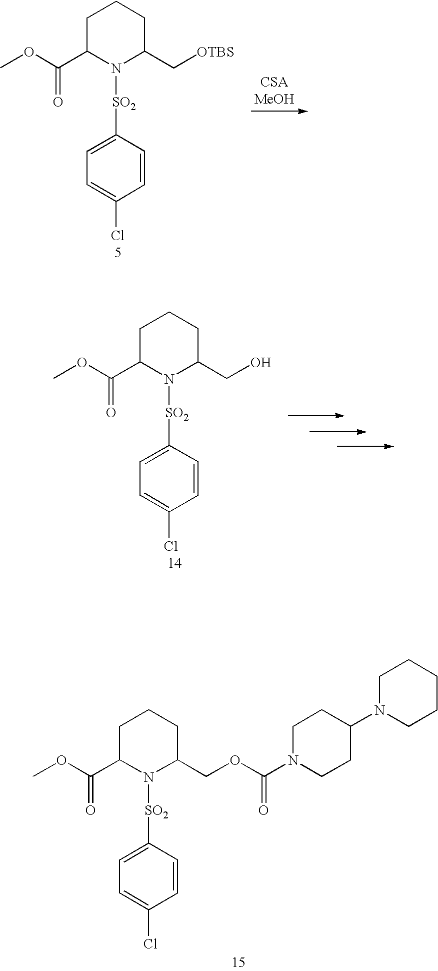

Step 1: A mixture of 480 mg (1.04 mmol) of Compound 5,10 ml of MeOH and 1 ml of DCM was warmed with a heat gun till dissolution was complete. Cooled to r.t., added 48 mg of CSA. Stirred for 1.5 h, diluted with DCM, washed with sat. NaHCO₃, dried over sodium sulfate and concentrated. The product was purified by chromatography using 30% of ethyl acetate in hexanes as solvent to furnish 320 mg of Compound 14.

Step 2. Compound 15 was prepared from Compound 14 using procedures similar to Example 1, Step 4(a) and 4(b), except that step 4(a) was modified so that a 2:1 mixture of THF and acetonitrile was used as solvent instead of DCM.

¹H NMR (CDCl₃ 400 MHz) δ 7.86 (2H, d, J=8.8 Hz), 7.63 (1H, s), 7.51 (2H, d, J=8.8 Hz), 7.09 (1H, s), 5.32 (1H, d, J=5.0 Hz), 4.25 (1H, m), 4.14 (1H, br), 3.73 (1H, t, J=9.0 Hz), 3.58 (1H, t, J=9.0 Hz), 2.70 (2H, m), 2.52-2.33 (6H, ser. m.), 2.0-1.2 (16H, ser. m.); MS (ES) m/e 542.3 (M⁺).

### Example 167

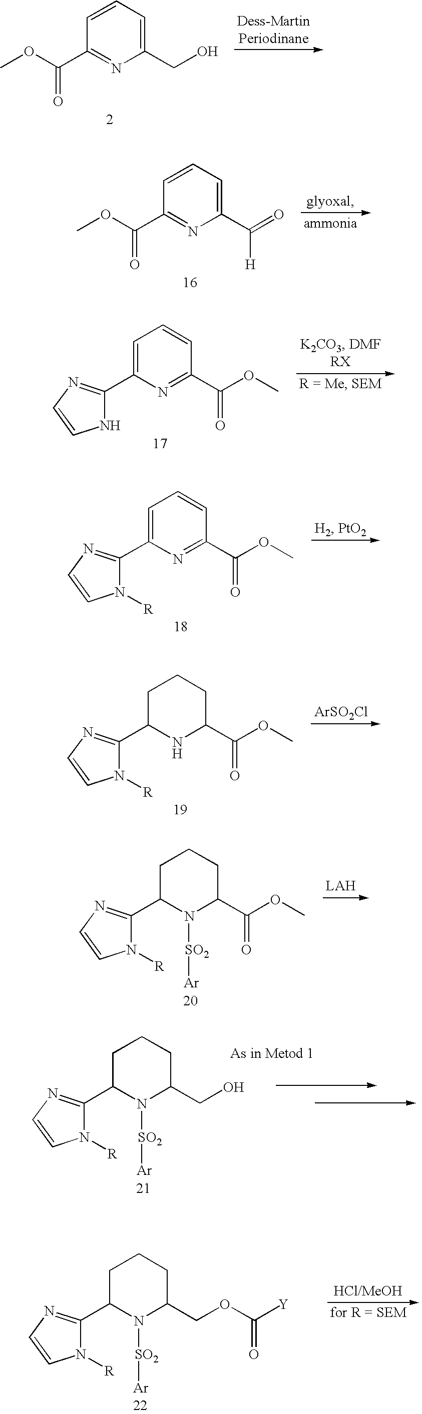

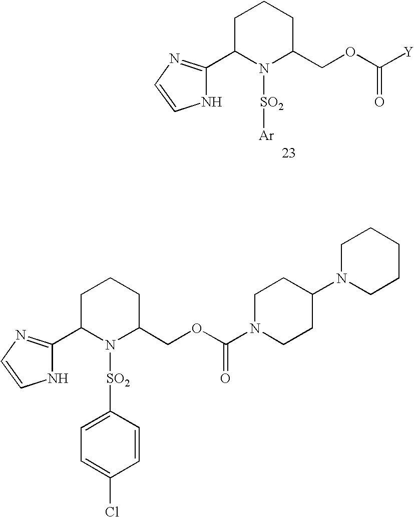

Step 1: Compound 2 was oxidized with Dess-Martin Periodinane using procedure similar to the one used in preparation of Compound 10.

Step 2: To a solution of 3.1 g (18.8 mmol) of Compound 16 in 95 ml of MeOH was added 7.9 g (37.5 mmol) of glyoxal trimer dihydrate followed by slow addition of 24.1 ml of 7 N ammonia/methanol solution. Work-up involved evaporation of volatiles and partitioning the residue between water and DCM. The aqueous phase was extracted with DCM, combined organic phase dried to yield 81.6 g of compound 17.

Step 3: To a solution of 250 mg (1.19 mmol) of Compound 17 in 7 ml of DMF was added 412.8 mg (2.99 mmol) of K₂CO₃ followed by 0.422 ml (2.4 mmol) of SEMCl. The mixture was stirred overnight, partitioned between water and DCM, aqueous phase was re-extracted with DCM, combined organic phase was dried over sodium sulfate, concentrated and purified chromatographically to furnish 230 mg of Compound 18.

Step 4: A mixture of 230 mg (0.69 mmol) of Compound 18, 40 mg of PtO₂, 10 ml of MeOH and 5 ml of AcOH was hydrogenated at 55 psi over a period of 15 hrs. The catalyst was filtered out, volatiles evaporated, residue dissolved in DCM and washed with sat. NaHCO₃, aqueous phase was re-extracted with DCM, combined organic phase was dried over sodium sulfate and concentrated to furnish Compound 19.

Step 5: Compound 20 was prepared from compound 19 using the procedure similar to the procedure used for the preparation of compound 5 in step 4 of example 165.

Step 6: Compound 21 was prepared from Compound 20 by reduction with LAH using the procedure described in Example 53, Preparation B. Step 4

Step 7: Compound 22 was prepared from Compound 21 using procedures similar to Example 1, Step 4(a) and 4(b), except that step 4(a) was modified so that a 2:1 mixture of THF and acetonitrile was used as solvent instead of DCM.

Step 8: A solution of compound 22 in 3M HCl/EtOH was refluxed for 3 hours, concentrated, partitioned between DCM and 15% aq. NaOH, aqueous phase was re-extracted with DCM, combined organic phase was dried over sodium sulfate, concentrated and purified chromatographically using 8% MeOH in DCM to furnish Compound 23.

¹H NMR (CDCl₃ 300 MHz) δ 10 (1H, s), 7.81 (2H, d, J=8.8 Hz), 7.53 (2H, d, J=8.8 Hz), 7.02 (2H, s), 4.48 (1H, d, J=4.8 Hz), 4.49 (1H, m), 4.20 (2H, d, J=12.0 Hz), 3.85 (1H, s), 3.38 (1H, t, J=10.4 Hz), 2.92-2.48 (7H, ser. m.), 2.06-1.17 (16H, ser. m.); MS (ES) m/e 550.1 (M+H)⁺.

Other compounds prepared by this method:

TABLE 18

| Compound | | Retention Time | Observed |
|---|---|---|---|
| No. | Structure | (minutes) | Mass |
| 167-A | 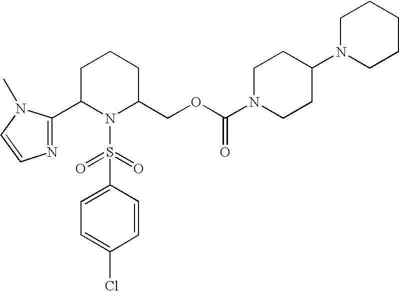 | 3.91 | 564.3 |
| 167-B | 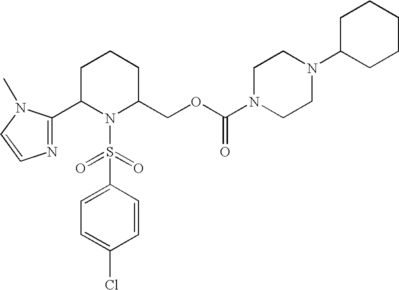 | 4.68 | 564.1 |
| 167-C | 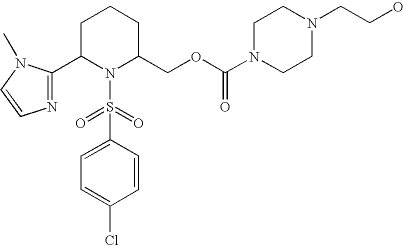 | N/A | N/A |

### Example 168

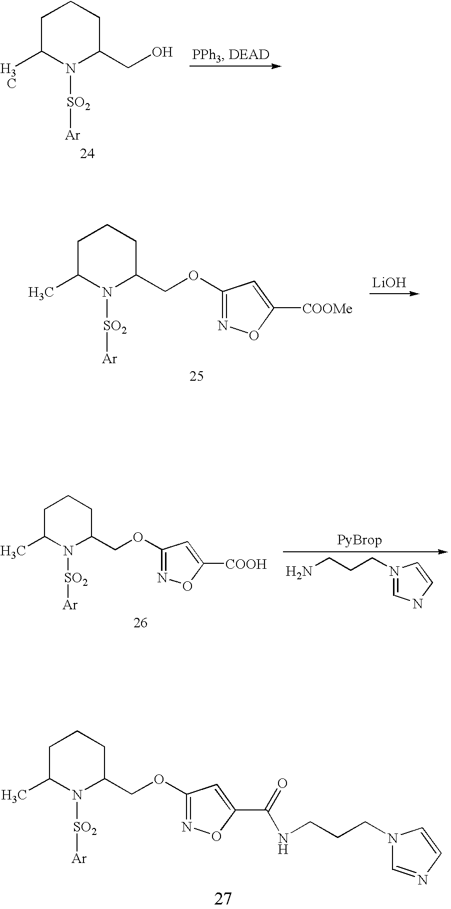

Step 1: To a mixture of 100 mg (0.329 mmol) of Compound 24, prepared as described in Example 1, in 1 ml of THF was added 172 mg (0.658 mmol) of triphenylphosphine and 114 mg (0.658 mmol) of DEAD. The mixture was stirred overnight, concentrated and chromatographed to yield 60 mg of Compound 25.

Step 2: To a solution of 60 mg of Compound 25 in 2 ml of THF was added a solution of 40 mg of LiOH in 0.3 ml of water. The mixture was stirred vigorously over a period of 4 hr, diluted with a few ml of 20% citric acid and extracted with DCM. The organic phase was dried over Na₂SO₄ and concentrated, the residue was passed through a silica gel plug using 10% of MeOH in DCM as solvent to yield 40 mg of Compound 26.

Step 3: A solution of 20 mg of Compound 26 in a mixture of 1 ml of DCM and 0.5 ml of DMF was treated with 20 mg of N-(3-aminopropyl)imidazole and 25 mg of PyBrop. The mixture was stirred overnight, washed with water, dried, concentrated and purified chromatographically using 10% of MeOH in DCM to furnish 12 mg of Compound 27.

¹H NMR (CDCl₃ 300 MHz) δ 7.78 (2H, d, J=8.8 Hz), 7.53 (1H, s), 7.47 (2H, d, J=8.8 Hz), 7.10-6.98 (2H, ser.m.), 6.52 (1H, s), 4.43-4.34 (3H, ser.m.), 4.14 (1H, m), 4.05 (2H, t, J=7.0 Hz), 3.44 (2H, m), 2.12 (3H, m), 1.90-1.20 (6H, ser.m.), 1.28 (3H, d, J=7.1 Hz); MS (ES) m/e 522.1 (M+H)⁺.

Other compounds prepared by this method:

TABLE 19

| Compound | | Retention Time | Observed |
|---|---|---|---|
| No. | Structure | (minutes) | Mass |
| 168-A | 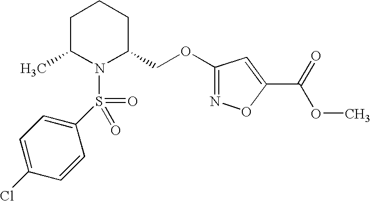 | 5.31 | 429.1 |

### Example 169

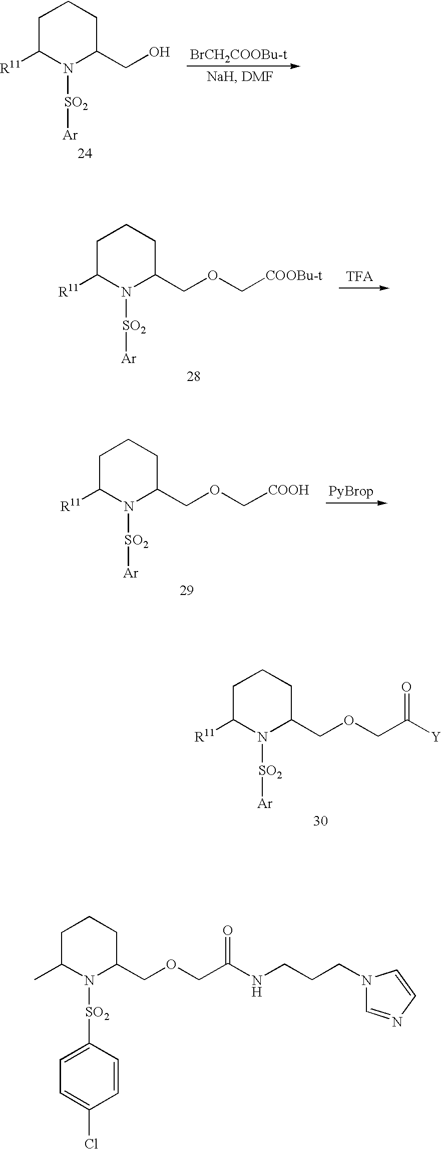

Step 1: To a solution of 100 mg (0.329 mmol) of Compound 24 in 1 ml of DMF was added 26 mg (0.658 mmol) of a 60% dispersion of NaH in mineral oil. The mixture was sonicated for 15 min. 137 mg (0.9 mmol) of t-butyl bromoacetate was added and the mixture was stirred overnight. Reaction was quenched with water, extracted with DCM, concentrated, passed through a silica gel plug using 10% of ethyl acetates in hexanes as solvent to furnish 130 mg of Compound 28.

Step 2: Dissolved 120 mg of compound 28 in 2 ml of DCM. Added 2 ml of TFA. Stirred the mixture for 30 min, evaporated volatiles. Obtained 120 mg of crude acid 29.

Step 3: For the preparation of amide 30 used the procedure described in Example 168 (synthesis of Compound 27).

¹H NMR (CDCl₃ 300 MHz) δ 7.76 (2H, d, J=8.8 Hz), 7.66 (1H, s), 7.48 (2H, d, J=8.8 Hz), 7.03 (2H, d, J=10.5 Hz), 4.40 (1H, m), 4.12-3.93 (4H, ser. m.), 3.83 (1H, m), 3.71 (1H, m), 3.52 (1H, m), 3.36 (2H, m), 2.65 (1H, br), 2.07 (2H, m), 1.66-1.26 (6H, ser. m.), 1.33 (3H, d, J=7.1 Hz); MS (ES) m/e 469.1 (M+H)⁺.

Other compounds prepared by this method:

TABLE 20

| Compound | | Retention Time | Observed |
|---|---|---|---|
| No. | Structure | minutes | Mass |
| 169-A | 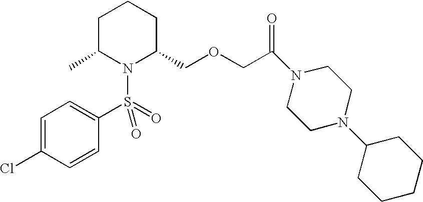 | 4.81 | 512.1 |
| 169-B | 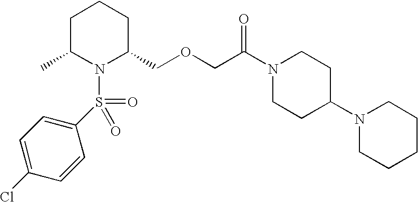 | 4.57 | 512.1 |
| 169-C | 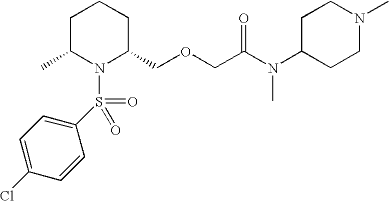 | 4.56 | 472.1 |
| 169-D | 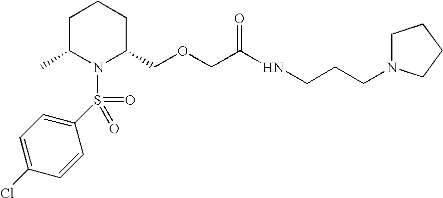 | 4.81 | 472.1 |

### Example 170

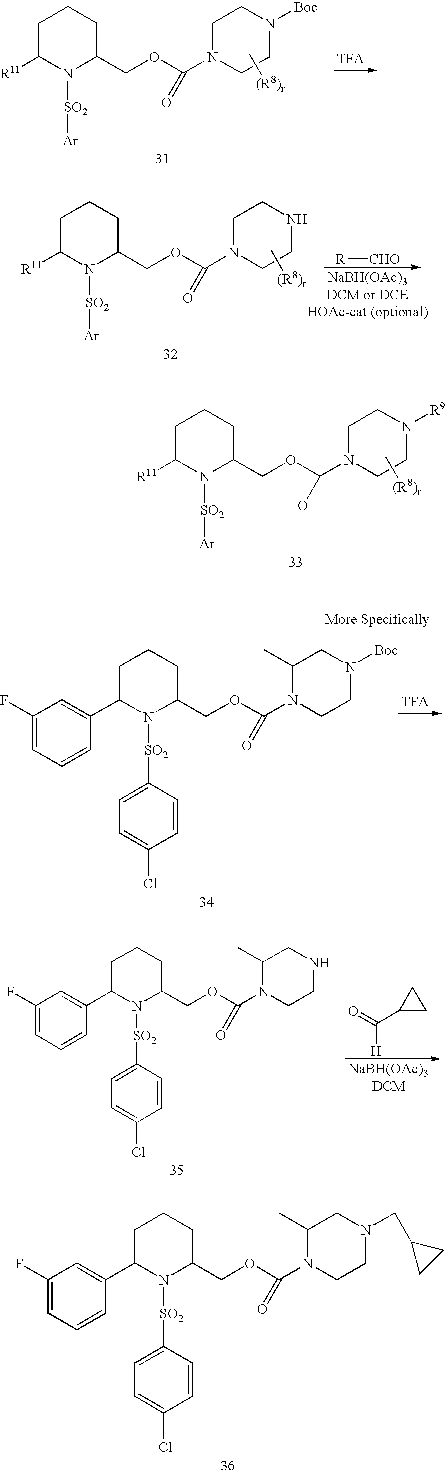

Step 1: 120 mg of Compound 34, prepared using procedures described in Example 53, was dissolved in 20 ml of DCM and treated with a pre-mixture of 10 ml of TFA and 1 ml of water. Reaction mixture was stirred over a period of 1 hr, volatiles were evaporated, residue was re-dissolved in DCM and washed with 1M sodium hydroxide. Organic phase was dried over sodium sulfate and concentrated to furnish 90 mg of Compound 35.

Step 2: To a solution of 44 mg (0.0864 mmol) of compound 35 in 2 ml of DCM was added 100 mg of cyclopropylcarboxaldehyde, 55 mg (0.259 mmol) of sodium triacetoxyborohydrate and one drop of acetic acid. The mixture was stirred overnight, diluted with DCM, washed with 1 M sodium hydroxide, dried over sodium sulfate and concentrated. The residue was purified by chromatography using 5% of MeOH in DCM as solvent. ¹H NMR (CDCl₃ 400 MHz) δ 7.85 (2H, m), 7.53 (2H, m), 7.38-7.27 (3H, m), 7.00-6.94 (1H, m), 5.19 (1H, m), 4.42-4.24 (2H, ser. m.), 3.91 (1H, m), 3.76 (1H, m), 3.50-3.38 (1H, m), 3.21 (1H, m), 2.89 (2H, m), 2.33-1.95 (4H, ser. m.), 1.64-1.20 (9H, ser. m., J=7.1 Hz), 0.85 (1H, ser. m.), 0.52 (2H, s), 0.11 (2H, s); MS (ES) m/e 564.1 (M+H)⁺.

Other compounds prepared are shown below:

TABLE 21

| Compound | | Retention Time | Observed |
|---|---|---|---|
| No. | Structure | (minutes) | Mass |
| 170-A | 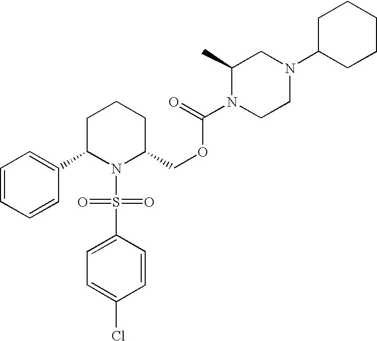 | 5.56 | 574.1 |
| 170-B | 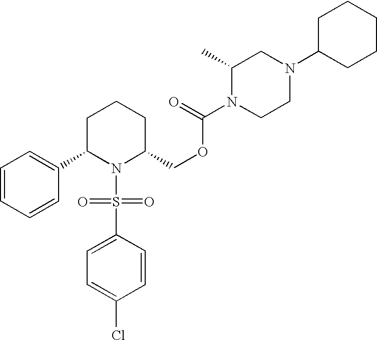 | 5.41 | 575.1 |
| 170-C | 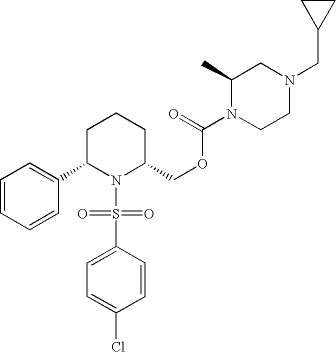 | 5.21 | 546.1 |
| 170-D | 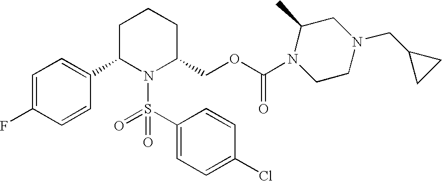 | 5.36 | 564.1 |
| 170-E | 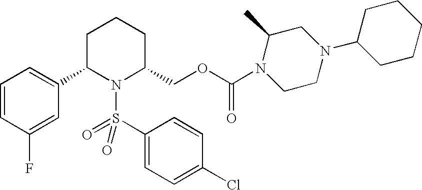 | 5.21 | 592.3 |
| 170-F | 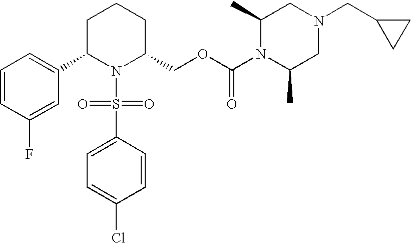 | 5.18 | 578.1 |
| 170-G | 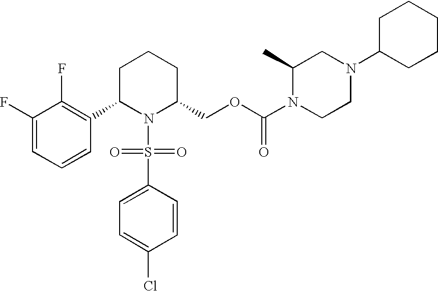 | 5.55 | 610.1 |
| 170-H | 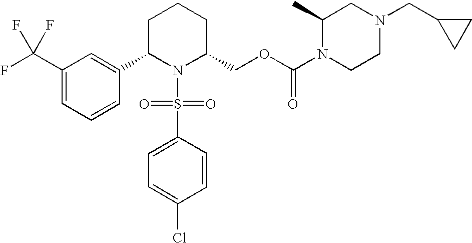 | 5.72 | 614.1 |
| 170-I | 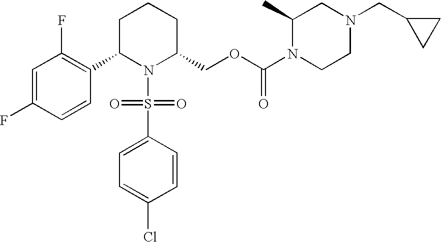 | 5.55 | 582.1 |
| 170-J | 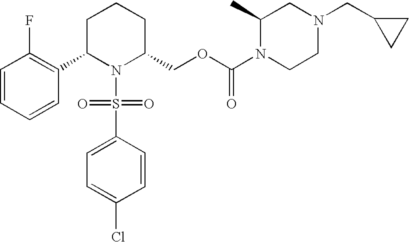 | 5.58 | 564.1 |
| 170-K | 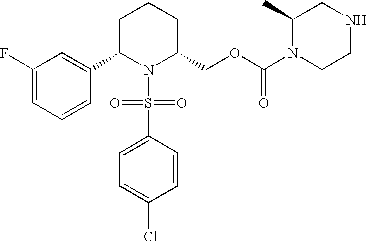 | 5.12 | 510.1 |
| 170-L | 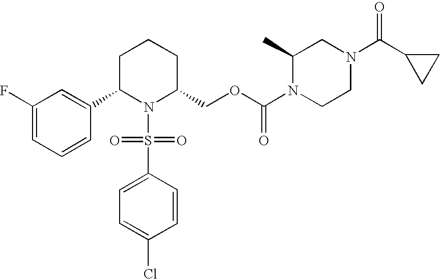 | 5.58 | 578.1 |
| 170-M | 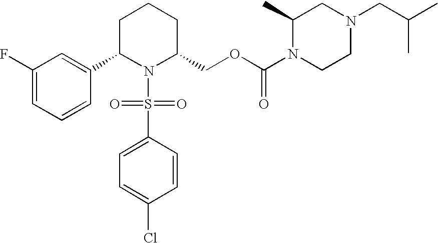 | 5.72 | 566.1 |
| 170-N | 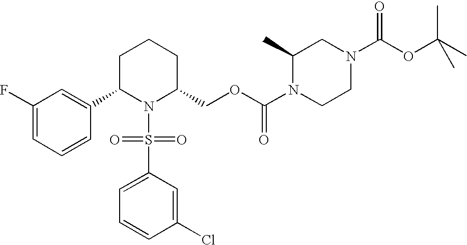 | 6.05 | 610.1 |
| 170-O | 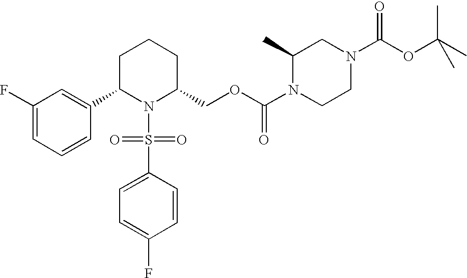 | 6.05 | 594.1 |
| 170-P | 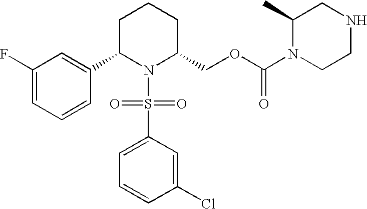 | 5.22 | 510.1 |
| 170-Q | 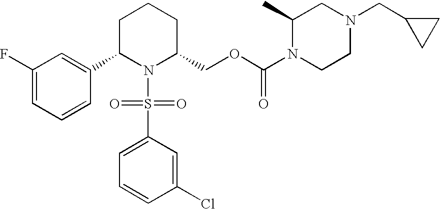 | 4.87 | 564.3 |
| 170-R | 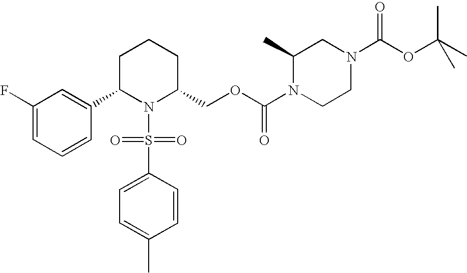 | 5.48 | 590.3 |
| 170-S | 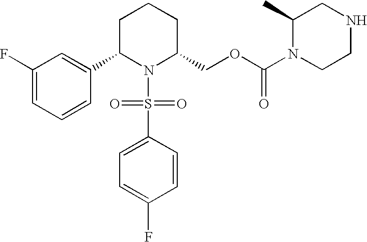 | 4.41 | 494.3 |
| 170-T | 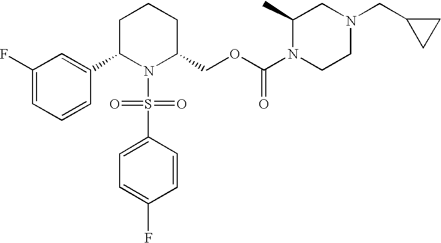 | 4.78 | 548.3 |
| 170-U | 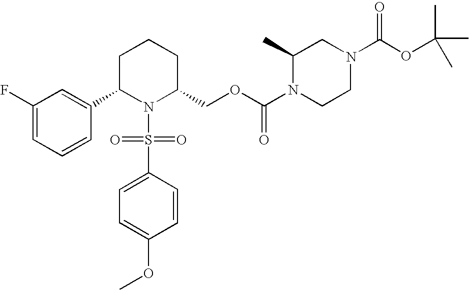 | 5.98 | 606.1 |
| 170-V | 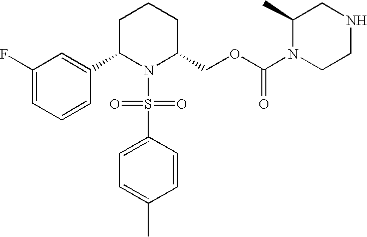 | 4.75 | 490.1 |
| 170-W | 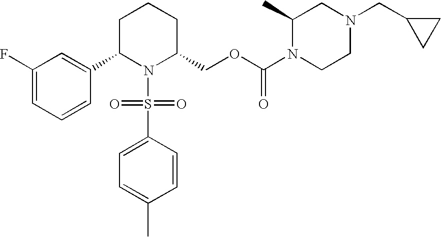 | 5.38 | 544.1 |
| 170-X | 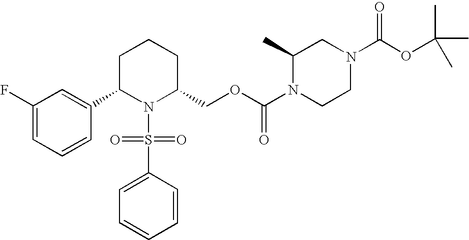 | 5.92 | 576.1 |
| 170-Y | 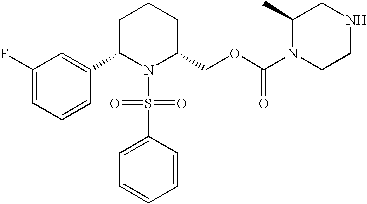 | 4.61 | 476.1 |
| 170-Z | 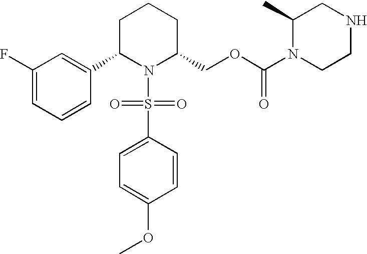 | 4.51 | 506.1 |
| 170-AA | 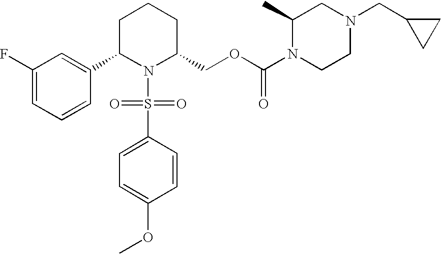 | 5.28 | 560.1 |
| 170-AB | 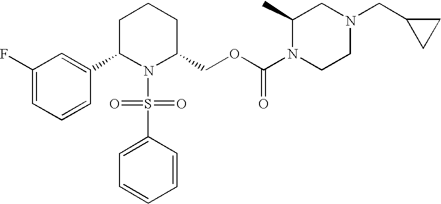 | 5.12 | 531.1 |
| 170-AC | 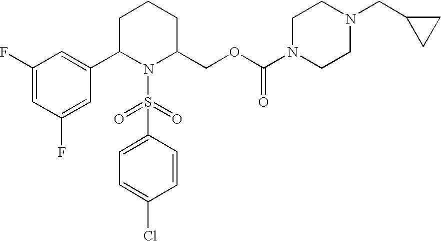 | 5.55 | 568.3 |
| 170-AD | 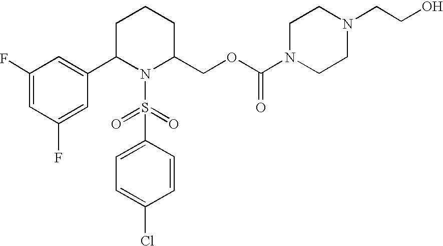 | 5.01 | 558.3 |

### Example 171

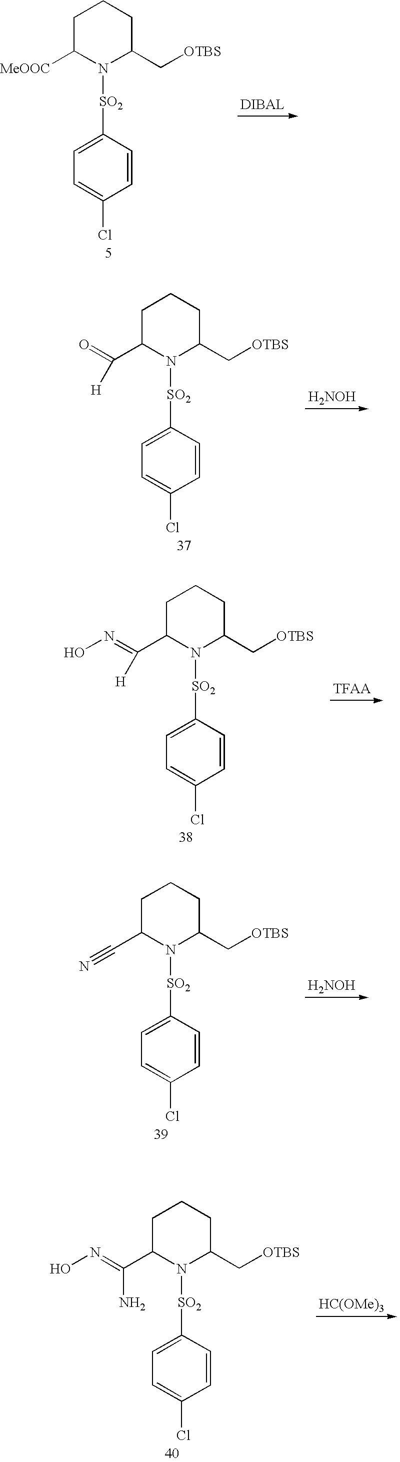

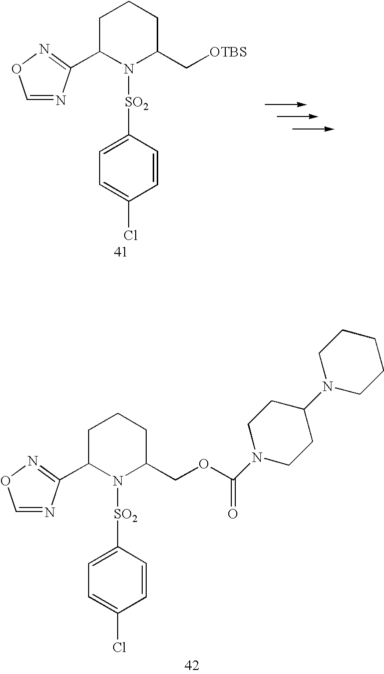

Step 1: To a solution of 1.35 g (2.92 mmol) of Compound 5 in 20.0 ml of DCM at −78° C. was added 3.2 ml (3.2 mmol) of 1 M solution of DIBAL in toluene. The mixture was stirred for 5 min, quenched with a 20% aq. sodium potassium tartrate solution, warmed up to room temperature, extracted with DCM, dried over sodium sulfate and concentrated. The product was purified chromatographically using DCM as solvent to furnish 1.06 g of aldehyde 37.

Step 2: A mixture of 3.21 g of aldehyde 37, 3.21 g of hydroxylamine hydrochloride, 8 ml of triethylamine and 50 ml of ethanol was heated briefly with a heat gun to boiling till all components dissolved. The reaction mixture was stirred overnight at r.t., volatiles were evaporated, residue partitioned between DCM and water, aqueous phase was re-extracted with DCM. Combined organic phase was dried over sodium sulfate and concentrated. The product was purified chromatographically using gradient 5 to 20% of ethyl acetate in hexanes as solvent to furnish 1.546 g of oxime 38.

Step 3: To a solution of 1.21 g (2.71 mmol) of oxime 38 in 12 ml of DCM was added 2.18 ml (27 mmol) of pyridine followed by 1.14 g (5.42 mmol) of trifluoroacetic acid. The reaction mixture was stirred for 1 h, washed with water, dried over sodium sulfate and concentrated. The product was purified chromatographically using 10% of ethyl acetate in hexanes as solvent to furnish 1.09 g of nitrile 39.

Step 4: Heated a mixture of 100 mg of nitrile 39, 100 mg of hydroxilamine hydrochloride, 0.1 ml of Hunig's base and 1.0 ml of ethanol at 80° C. for 10 min, removed heating and stirred over 24 h. The reaction mixture was partitioned between water and DCM, organic phase was dried over sodium sulfate and concentrated. The product was purified chromatographically using 30% of ethyl acetate in hexanes as solvent to furnish 90 mg of amidoxime 40.

Step 5: A mixture of 90 mg of amidoxime 40, 3.0 ml of triethylorthoformate, 5 mg of tosic acid hydrate and 0.5 ml of DCM was heated at 100° C. over a period of 40 min. 5 The reaction mixture was partitioned between DCM and sat. sodium bicarbonate, organic phase was dried over sodium sulfate and concentrated. The product was purified chromatographically using 20% of ethyl acetate in hexanes as solvent to furnish 70 mg of oxadiazole 41.

Step 6: Conversion of oxadiazole 41 to compound 42 was carried out according to Steps 1 and 2 of example 166. ¹H NMR (CDCl₃ 300 MHz) δ 8.67 (1H, s), 7.89 (2H, d, J=8.05 Hz), 7.50 (2H, d, J=8.05 Hz), 5.42 (1H, d, J=5.8 Hz), 4.26 (1H, m), 4.12 (2H, m), 3.83 (2H, m), 2.69 (2H, m), 2.48 (4H, m), 2.37 (2H, m), 1.84-1.36 (15H, ser. m.), MS (ES) m/e 552.1 (M+H)⁺.

### Example 172

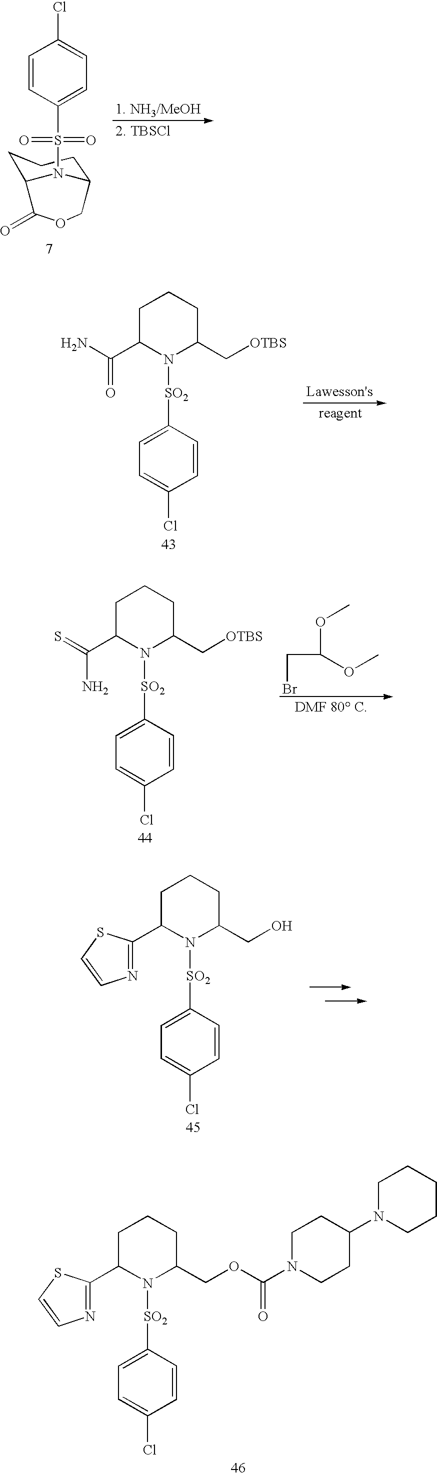

Step 1: Stirred a mixture of 1.0 g of compound 7 in 10 ml of 7 M solution of ammonia in methanol over a period of 3 h and evaporated the volatiles. 500 mg of resulting product was dissolved in 5 ml of DMF and treated with 152 mg (2.24 mmol) of imidazole and 218 mg (1.456 mmol) of TBSCl. Reaction mixture was stirred overnight, diluted with DCM, washed with sat. NaHCO₃, dried and concentrated. The product was purified chromatographically using 20% of ethyl acetate in hexanes as solvent to furnish 500 mg of amide 43.

Step 2: A mixture of 250 mg (0.56 mmol) of amide 43 and 226 mg (0.56 mmol) of Lawesson's reagent was refluxed in 3 ml of DCM over 8 h. Solvent was evaporated and the product purified by prep. TLC using 30% of ethyl acetate in hexanes as solvent to furnish 70 mg of thioamide 44.

Step 3: Heated a mixture of 70 mg (0.151 mmol) of thioamide 44, 0.5 ml of dimethylacetal of bromoaldehyde in 1 ml of DMF at 80° C. over a period of 5 h. Reaction mixture was partitioned between DCM and sat. NaHCO₃, dried and concentrated. The product was purified chromatographically using 30% of ethyl acetate in hexanes as solvent to furnish 25 mg of thiazole 45.

Step 4: Transformation of alcohol 45 to compound 46 was carried out according to Example 1 steps A and B. LCMS m/z=567.1, retention 4.88 min. ¹H NMR (CDCl₃ 300 MHz) δ 7.86 (2H, d, J=8.8 Hz), 7.68 (1H, d, J=3.3 Hz), 7.52 (2H, d, J=8.8 Hz), 7.37 (1H, d, J=3.3 Hz), 5.35 (1H, d, J=5.5 Hz), 4.36 (1H, m), 4.20 (2H, m), 3.83 (1H, dd, J=6.6, 11.0 Hz), 3.63 (1H, dd, J=8.7, 11.0 Hz), 2.82-2.33 (8H, ser. m.), 1.88-1.20 (15H, ser. m.), MS (ES) m/e 567.1 (M+H)⁺.

### Example 173

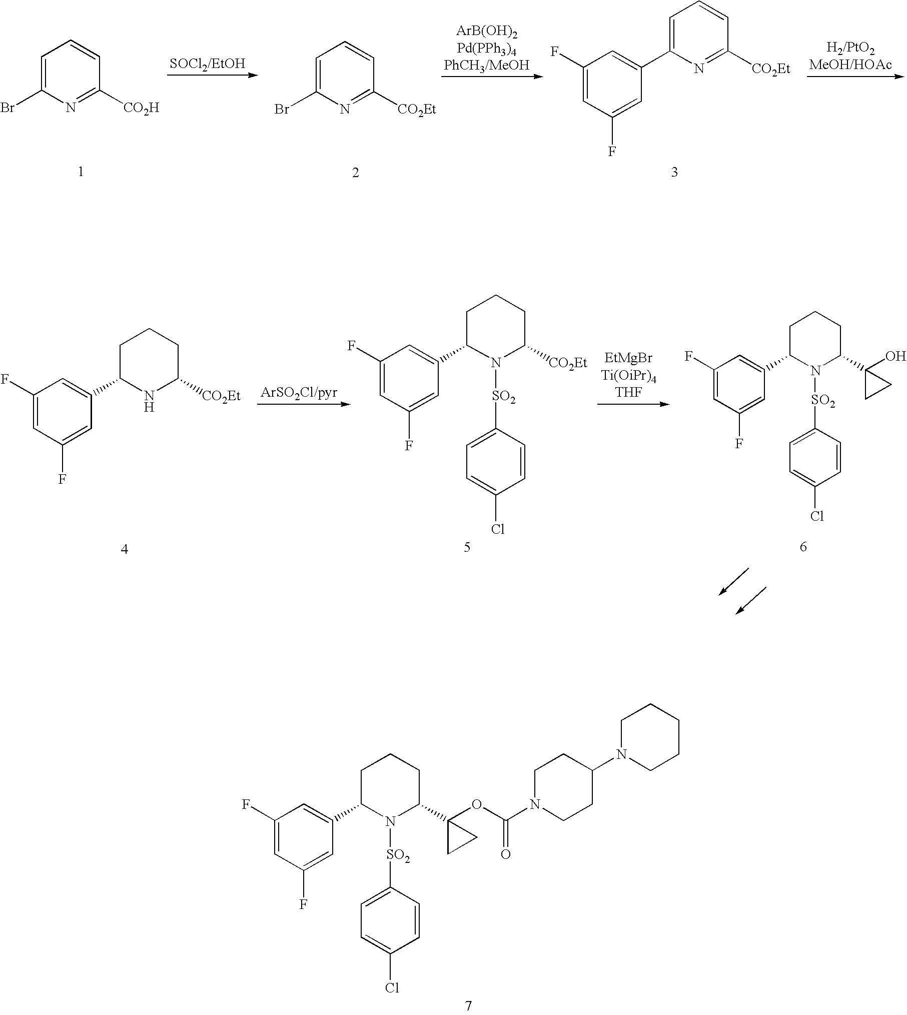

Step 1: To a stirring solution of 6-bromopicolinic acid (14.25 g, 70.3 mmol) in anhydrous ethanol (250 ml) is slowly added thionyl chloride (60 ml) at 5° C. After the addition is completed, remove the ice-bath and stir the mixture at 25° C. for 3 hr. Evaporate the solvent in vacuo, basify aqueous residue with saturated sodium carbonate, and extract with DCM. Dry the organic phase over Na₂SO₄ and concentrate to give ethyl 6-bromopicolinate as white solid (15.75 g).

Step 2: Heat ethyl 6-bromopicolinate (15.75 g, 68.5 mmol), 3,5-difluorophenylboronic acid (12.98 g, 82.2 mmol), tetrakis(triphenylphsphine)palladium (7.9 g, 6.85 mmol) and sodium carbonate (18 g) in toluene (160 ml) and methanol (80 ml) under reflux for 16 hr. Cool to room temperature, dilute with DCM, and filter. Wash the filtrate with water, concentrate the dried (Na₂SO₄) organic solution, and purify the residue chromatographically using 5% ethyl acetate in hexanes to give 10.6 g of the product, as white solid.

Step 3: Under a hydrogen atmosphere, stir a solution of Compound 3 (10.5 g, 39.9 mmol) in methanol (400 ml) and glacial acetic acid (40 ml) in the presence of platinum oxide (1.81 g) for 72 hr. Purge the reaction mixture with nitrogen. Filter and then concentrate the reaction mixture in vacuo. Take up the residue in water, basify with saturated sodium carbonate, and extract with DCM. Dry the organic phase over Na₂SO₄ and concentrate in vacuo to give light yellow foam (10.7 g).

Step 4: A solution of Compound 4 (10.7 g, 39.7 mmol) in pyridine (100 ml) is treated with 4-chlorobenzenesulfonylchloride (16.8 g, 79.5 mmol). The mixture is heated at 60° C. for 4 hr. Cool to room temperature, concentrate in vacuo, and the residue is subjected to flash-chromatography over silica gel (eluting 10% ethyl acetate in hexanes) to provide 14 g of product, as white powder.

Step 5: To a stirring solution of Compound 5 (2.0 g, 4.5 mmol) and titanium isopropoxide (0.41 ml, 1.35 mmol) in terahydrofuran (15 ml) is added a solution of ethylmagnesium bromide (4.5 ml, 13.5 ml, 3M in Et₂O) slowly over a period of 1 hr at 5° C., and the stirring is continued for 10 min. The mixture is then poured into cooled (5° C.) 10% aq HCl (45 ml) and the products are extracted with DCM (3×25 ml). The combined DCM extracts are washed with water (25 ml), dried (Na₂SO₄), and the solvent is removed. The product is obtained by flash-chromatography (eluting 13% ethyl acetate in hexanes) as light yellow oil (1.5 g).

Step 6: The compound was prepared from Compound 6 using procedures similar to Example 1, Step 4(a) and 4(b), except that step 4(a) was modified so that a 2:1 mixture of THF and acetonitrile was used as solvent instead of DCM, and the mixture was heated at 78° C. for 16 hr.

¹H NMR (CDCl₃, 400 MHz) δ 7.81 (2H, d, J=8.3 Hz), 7.79 (2H, d, J=7.9 Hz), 7.49 (2H, d, J=8.1 Hz), 6.75-6.62 (1H, m), 5.50-4.60 (2H, m), 4.35-3.62 (2H, m), 2.90-2.20 (7H, m), 2.10-0.86 (16H, m), 0.85-0.63 (2H, m), 0.50-0.10 (2H, m); MS (ES) m/e 623.1 (M+H)⁺.

Compounds prepared via a similar method:

TABLE 22

| Compound | | Retention Time | |
|---|---|---|---|
| No. | Structure | (minutes) | Observed Mass |
| 173-A | 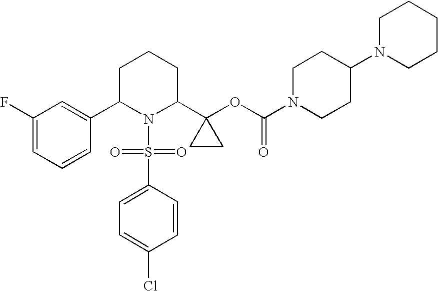 | 5.45 | 604.1 |
| 173-B | 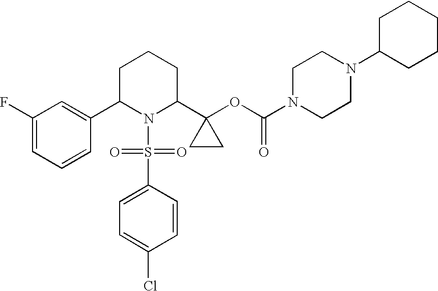 | 5.55 | 604.1 |
| 173-C | 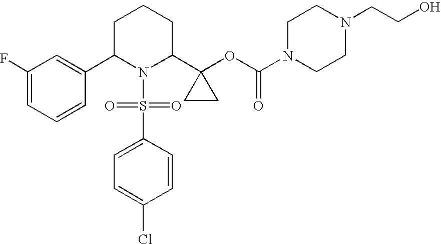 | 4.95 | 566.1 |
| 173-D | 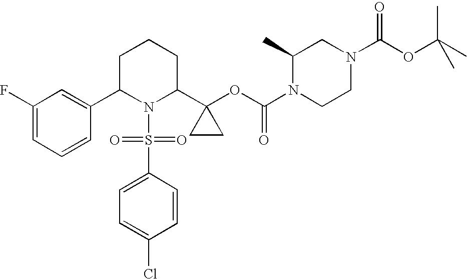 | 5.62 | 636.2 |
| 173-E | 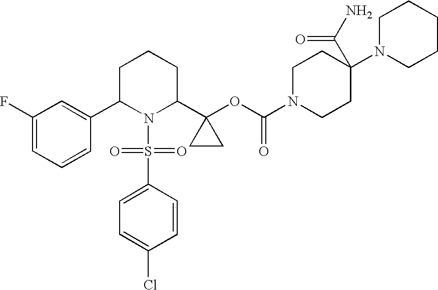 | 4.65 | 647.4 |
| 173-F | 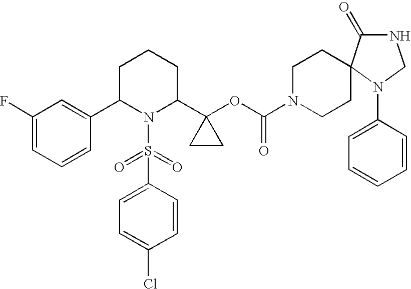 | 5.08 | 667.4 |
| 173-G | 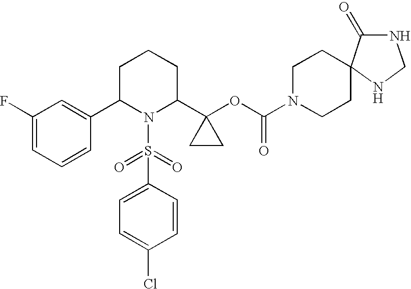 | 4.24 | 591.3 |
| 173-I | 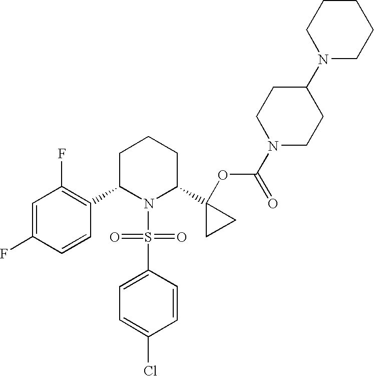 | 5.75 | 622.1 |
| 173-J | 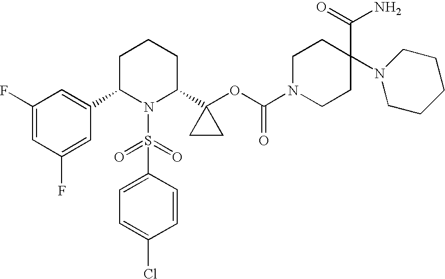 | 5.12 | 665.2 |
| 173-K | 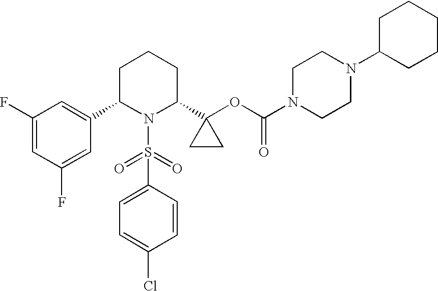 | 5.45 | 622.1 |
| 173-L | 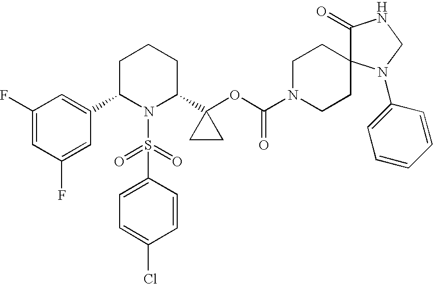 | 5.42 | 685.2 |
| 173-M | 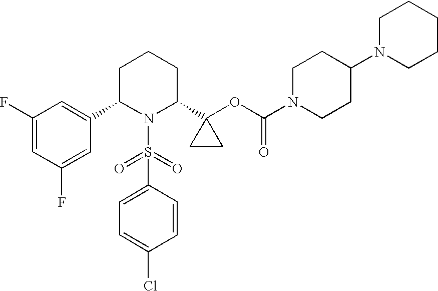 | 5.55 | 622.1 |
| 173-N | 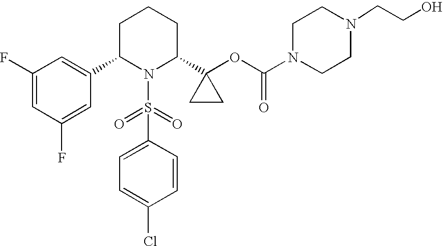 | 5.02 | 584.1 |
| 173-O | 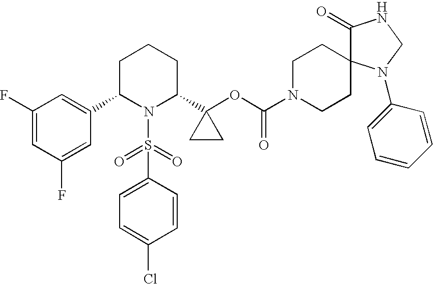 | 5.42 | 685.2 |
| 173-P | 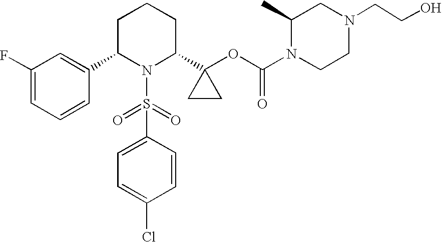 | 4.91 | 580.1 |
| 173-Q | 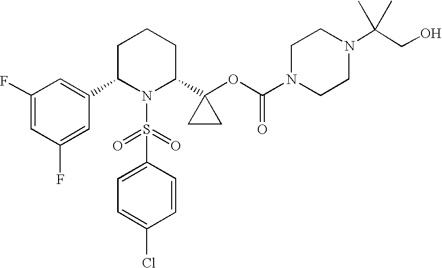 | 5.08 | 612.1 |
| 173-R | 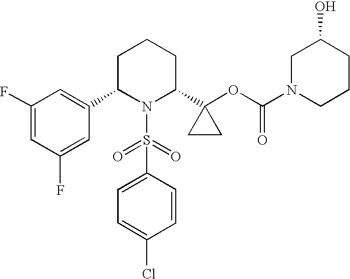 | 4.68 | 555.1 |
| 173-S | 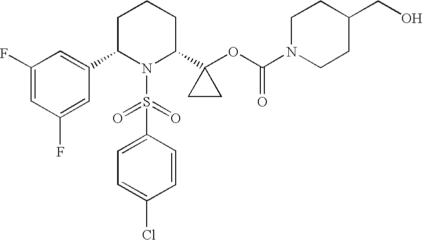 | 4.69 | 569.1 |
| 173-T | 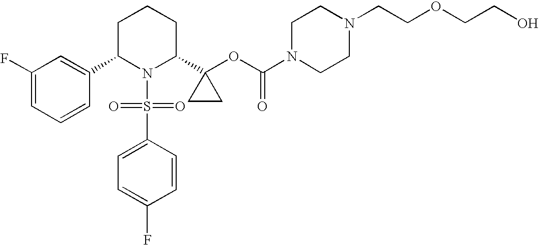 | 4.43 | 494.1 |
| 173-U | 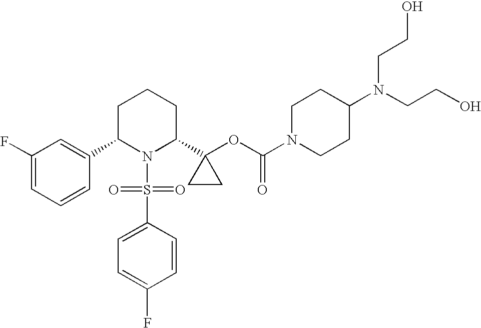 | 4.38 | 608.1 |

### Example 174

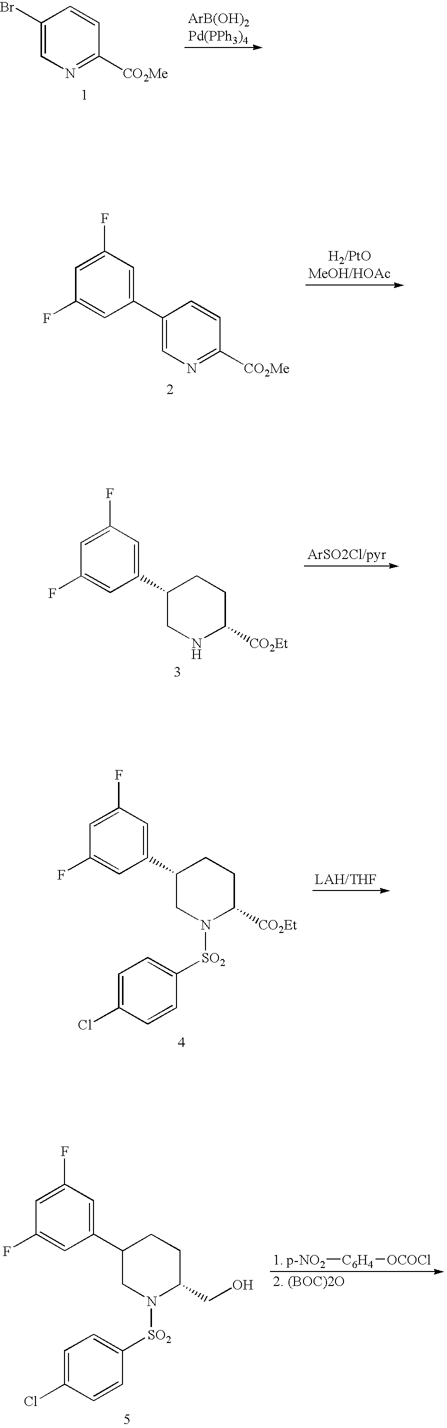

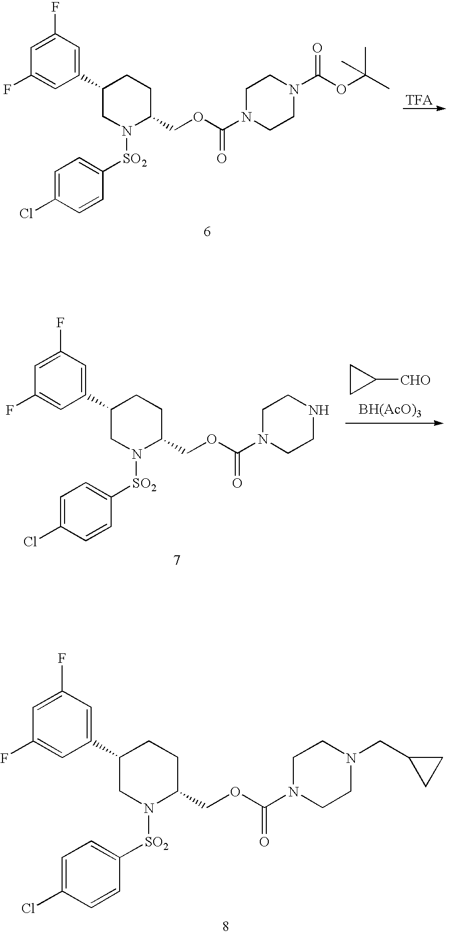

Step 1: Methyl 5-Bromopicolinate 1 was obtained as described in J. J. Song and N. K. Yee, J. Org. Chem. 2001, 66, 605-608. A solution of this ester (2.5 g, 11.6 mmol) in a mixture of toluene (160 ml) and ethanol (80 ml) is treated with 3,5-difluorobenzeneboronic acid (2.19 g, 13.9 mmol), tetrakis(triphenyphosphine)palladium (1.34 g, 1.16 mmol) and sodium carbonate (2.5 g). The mixture is heated at reflux for 16 hr. The solvent is removed at reduced pressure. The residue is redissolved in DCM, washed with water, dried over Na₂SO₄, concentrated and purified chromatographically using 30% ethyl acetate in hexanes as solvent to furnish 2.17 g of the product.

Step 2: Under a hydrogen atmosphere, stir a solution of Compound 2 (2.3 g, 9.2 mmol) in methanol (90 ml) and glacial acetic acid (10 ml) in the presence of platinum oxide (0.42 g) for 8 hr. Purge the reaction mixture with nitrogen. Filter and then concentrate the reaction mixture in vacuo. Take up the residue in water, basify with saturated sodium carbonate, and extract with DCM. Dry the organic phase over Na₂SO₄ and concentrate in vacuo to give light yellow foam (2.3 g).

Step 3: A solution of Compound 3 (2.3 g, 9.2 mmol) in pyridine (20 ml) is treated with 4-chlorobenzenesulfonylchloride (3.8 g, 18.5 mmol). The mixture is heated at 60° C. for 16 hr. Cool to room temperature, concentrate in vacuo, and the residue subjected to flash-chromatography over silica gel (eluting 10% ethyl acetate in hexanes) to provide 2.1 g of product, as white powder.

Step 4: To an ice-cold solution of Compound 4 (2.1 g, 4.9 mmol) in THF (15 ml) is slowly added a solution of lithium aluminum hydride (9.8 ml, 1M THF). The cooling bath is removed and the reaction is stirred at ambient temperature for 2 hr. The mixture is quenched sequentially with water (0.4 ml), 15% NaOH (0.4 ml), and water (1.2 ml). The mixture is stirred for 1 hr, filtered, the filtrate dried over Na₂SO₄, and concentrated to give 1.8 g of the product as yellow solid.

Step 5: This was prepared according to Step 4 of Example 1, using N-Boc piperazine at the last stage as the amine.

Step 6: A solution of Compound 6 (100.0 mg, 0.163 mmol) in DCM (3 ml) is treated with TFA, and the mixture is stirred at ambient temperature for 2 hr. The mixture is basified with saturated sodium carbonate, extracted with DCM, dried over Na₂SO₄, and concentrated to afford 72.3 mg of the product, as white powder.

Step 7: To a solution of Compound 7 (50.0 mg, 0.097 mmol) in dichloroethane (2.0 ml) is added cyclopropanecarboxaldehyde (20.0 mg, 0.28 mmol) followed by sodium triacetoxyborohydride (60.0 mg, 0.28 mmol) and one drop of acetic acid. After stirring at ambient temperature for 16 hr, the mixture is diluted with water and basified with saturated sodium carbonate. The crude product is extracted with DCM, washed with water, dried over Na₂SO₄, and concentrated. The crude was purified by preparative TLC (eluting 95:5:0.5; DCM:MeOH:NH₄OH) to furnish 30.0 mg of the product, as white powder. ¹H NMR (CDCl₃, 400 MHz) δ 7.78 (2H, d, J=7.8 Hz), 7.49 (2H, d, J=7.8 Hz), 6.75-6.62 (3H, m), 4.50-4.36 (2H, m), 4.18-4.02 (1H, m), 3.89-3.71 (1H, m), 3.52 (4H, s. br.), 3.08 (1H, t, J=9.0 Hz)), 2.65-2.34 (4H, m), 2.34 (2H, d, J=6.6 Hz), 1.84-1.56 (4H, m), 0.95-0.74 (1H, m), 0.53 (2H, d, J=7.8 Hz), 0.11 (2H, d, J=4.5 Hz); MS (ES) m/e 569.1 (M+H)⁺.

### Example 175

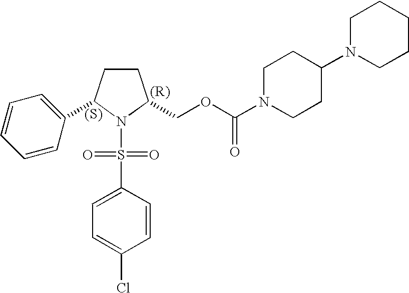

Step 1

(2R,5S)-Boc-5-phenyl-pyrrolidine-2-carboxylic acid (1.3 g, 4.5 mmol, obtained from SNPE North America LLC, 5 Vaughn Drive—Suite 111, Princeton, N.J. 08540, USA) was added to 10 mL of 4N HCl in dioxane and the mixture was stirred at room temperature for 2 hr. The mixture was concentrated in vacuo to give 1.0 g (100%) of (2R,5S)-5-phenyl-pyrrolidine-2-carboxylic acid HCl salt as a white solid. The solid was dissolved in 7 mL of anhydrous THF and the solution was added slowly into a stirred solution of 1 M LiAlH₄ in THF (10.3 mL, 10.3 mmol, 3 eq) at room temperature. The mixture was then heated at reflux for 4 hr. After cooling to room temperature, the reaction mixture was treated sequentially with 0.42 mL of water, 0.85 mL of 1N NaOH, and 1.26 mL of water. The mixture was stirred for 1 hr and the white precipitate was filtered off. The filtrate was dried (Na₂SO₄) and concentrated in vacuo to give 0.78 g (98%) of (2R,5S)-(5-phenyl-pyrrolidin-2-yl)-methanol as a yellow oil. 1H NMR (CDCl₃, 300 MHz) δ 7.60-7.30 (5H, m), 4.40 (1H, m), 3.78 (1H, m), 3.60 (2H, m), 2.60 (2H, br.s), 2.25 (1H, m), 2.10 (1H, m), 1.80 (2H, m).

Step 2

To a solution of (2R,5S)-(5-phenyl-pyrrolidin-2-yl)-methanol (0.78 g, 4.4 mmol) in CH₂Cl₂ (7 mL) at 0° C. was added 0.72 mL (5.2 mmol, 1.2 eq) of Et₃N followed by a slow addition of 0.66 mL (5.2 mmol, 1.2 eq) of TMSCl. The mixture was stirred at 0° C. for 45 min. Water (3 mL) was added to quench the reaction. The organic layer was separated and the aqueous layer was extracted with CH₂Cl₂ (2 mL×2). The combined organic layer was dried (Na₂SO₄) and concentrated in vacuo to give 0.98 g (89%) of (2R,5S)-2-phenyl-5-trimethylsilanyloxymethyl-pyrrolidine as a yellow oil. 1H NMR (CDCl₃, 300 MHz) δ 7.60-7.30 (5H, m), 4.30 (1H, m), 3.85 (2H, m), 3.50 (1H, m), 2.25 (1H, m), 2.05 (1H, m), 0.25 (9H, m).

Step 3

To a solution of (2R,5S)-2-phenyl-5-trimethylsilanyloxymethyl-pyrrolidine (0.98 g, 3.9 mmol) in ClCH₂CH₂Cl (5 mL) was added 1.9 mL (13.7 mmol, 3.5 eq) of Et₃N and 1.45 g (6.86 mmol, 1.8 eq) of 4-chlorobenzenesulfonyl chloride. The mixture was heated at 70° C. for 16 hr. After cooling to room temperature, the mixture was diluted with CH₂Cl₂ (20 mL), washed with water (10 mL), saturated brine (10 mL), dried (Na₂SO₄) and concentrated in vacuo. The crude product was chromatographed on silica gel (2% EtOAc/hexane) to give 0.78 g (46%) of (2R,5S)-1-(4-chlorobenzenesulfonyl)-2-phenyl-5-trimethylsilanyloxymethyl-pyrrolidine as a yellow gum. 1H NMR (CDCl₃, 300 MHz) δ 7.80 (2H, d), 7.55 (2H, d), 7.40 (5H, m), 4.72 (1H, t), 4.07 (2H, m), 3.85 (1H, m), 2.20-2.00 (3H, m), 1.82 (1H, m), 0.30 (9H, m).

Step 4

To a solution of (2R,5S)-1-(4-chloro-benzenesulfonyl)-2-phenyl-5-trimethylsilanyloxymethyl-pyrrolidine (0.76 g, 1.8 mmol) in 15 mL of MeOH at 0° C. was added K₂CO₃ (15 mg, 0.11 mmol, catalyst). After stirring at 0° C. for 30 min, the reaction mixture was partitioned between EtOAc (50 mL) and saturated brine (50 mL). The organic phase was separated, dried (Na₂SO₄) and concentrated in vacuo to give 0.63 g (100%) of (2R,5S)-[1-(4-chloro-benzenesulfonyl)-5-phenyl-pyrrolidin-2-yl]-methanol as a yellow gum. 1H NMR (CDCl₃, 300 MHz) δ 7.90 (1H, d), 7.60 (1H, d), 7.40 (5H, m), 4.87 (1H, t), 4.10-3.90 (3H, m), 2.90 (1H, m), 2.05 (2H, m), 1.85 (2H, m). HPLC analysis using an analytical chiracel OD column (hexane/isopropanol) showed an e.e. >99% for this compound (r.t. for (2R,5S)-eantiomer=9.9 min, r.t. for (2S,5R)-enantiomer=12.3 min).

Step 5

To a solution of (2R,5S)-[1-(4-chloro-benzenesulfonyl)-5-phenyl-pyrrolidin-2-yl]-methanol (0.040 g, 0.11 mmol) in 1 mL of CH₂Cl₂ was added 0.046 mL (0.33 mmol, 3 eq) of Et₃N followed by 0.022 g (0.11 mmol, 1 eq) of 4-nitrophenyl chloroformate. The mixture was stirred at room temperature for 16 hr. 4-Piperidinopiperidine (0.018 g, 0.11 mmol, 1 eq) was added and stirring continued for 6 hr. The mixture was diluted with 10 mL of CH₂Cl₂ and washed with 1N NaOH (5 mL×2), water (5 mL×2), and saturated brine (5 mL). The organic layer was dried (Na₂SO₄) and concentrated in vacuo to give a yellow gum. Purification using reverse phase prep-HPLC gave 0.030 g (51%) of the desired product (2R,5S)-[1,4′]bipiperidinyl-1′-carboxylic acid 1-(4-chloro-benzenesulfonyl)-5-phenyl-pyrrolidin-2-ylmethyl ester as a white solid. 1H NMR (CD₃OD, 300 MHz) δ 7.97 (2H, d), 7.75 (2H, d), 7.60-7.50 (5H, m), 4.80 (1H, t), 4.55-4.20 (3H, m), 3.65-3.40 (4H, m), 3.20-2.95 (4H, m), 2.30-1.60 (14H, m). MS(ESI): MH⁺=546.2.

### Example 176

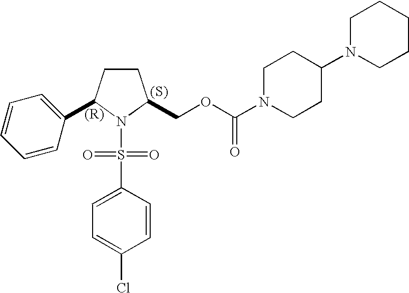

Following a similar procedure as in Example 175 except for using (2S,5R)-Boc-5-phenyl-pyrrolidine-2-carboxylic acid (1.3 g, 4.5 mmol, obtained from SNPE North America LLC, 5 Vaughn Drive—Suite 111, Princeton, N.J. 08540, USA) as the starting material, 0.035 g (59%) of (2S,5R)-[1,4′]bipiperidinyl-1′-carboxylic acid 1-(4-chloro-benzenesulfonyl)-5-phenyl-pyrrolidin-2-ylmethyl ester was obtained as a white solid. MS(ESI): MH⁺=546.2.

Following procedures similar to those in Example 175, the compounds in Table 24 were prepared.

TABLE 23

| | | Exact MS, | MS (ESI) MH⁺ |
|---|---|---|---|
| Compound | Structure | calc. | found |
| 176-A | 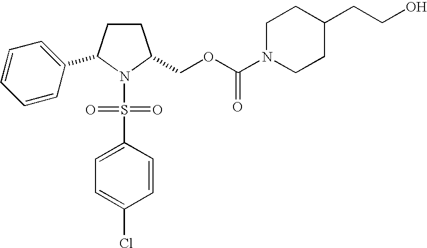 | 506.16 | 506.9 |
| 176-B | 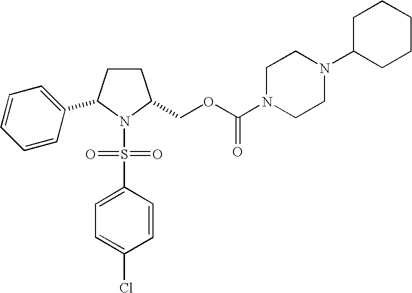 | 545.21 | 546.2 |
| 176-C | 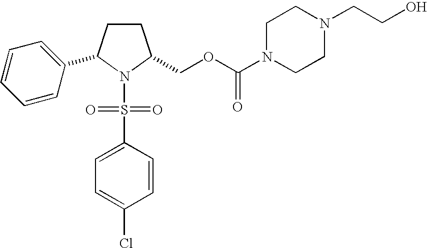 | 507.16 | 508.0 |
| 176-D | 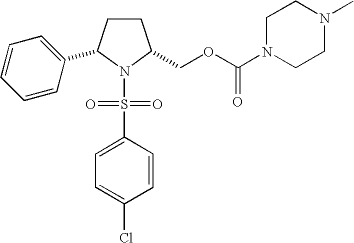 | 477.15 | 478.1 |
| 176-E | 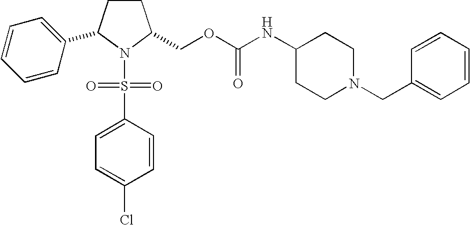 | 567.20 | 568.1 |
| 176-F | 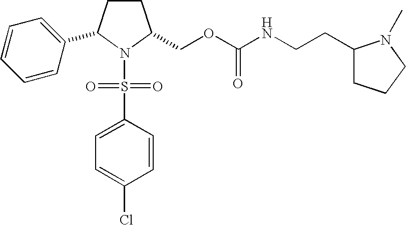 | 505.18 | 506.1 |
| 176-G | 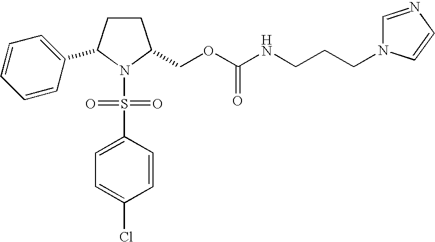 | 502.14 | 503.1 |
| 176-H | 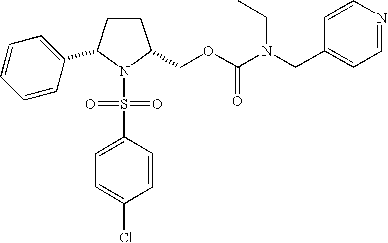 | 513.15 | 514.1 |
| 176-I | 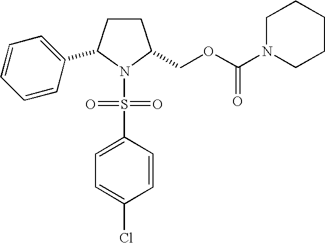 | 462.14 | 462.9 |
| 176-J | 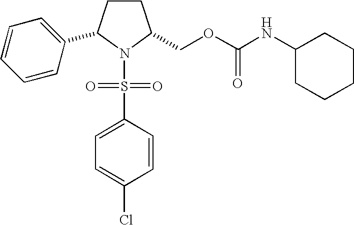 | 476.15 | 476.9 |

### Example 177

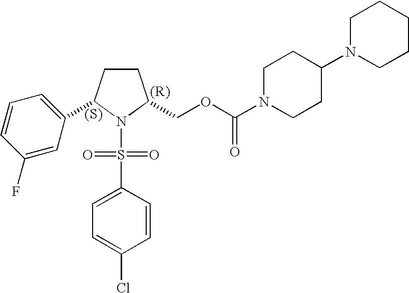

Step 1

Thionyl chloride (8.5 mL, 0.12 mol) was added dropwise into 40 mL of anhydrous MeOH at −20° C. D-Pyroglutamic acid (10 g, 0.077 mol, obtained from Aldrich, P.O. Box 2060, Milwaukee, Wis. 53201, USA) was added in one portion and the reaction mixture was stirred at to room temperature for 16 hr. The mixture was cooled to 0° C. and solid NaHCO₃ was added until pH reached about 9. The mixture was filtered through Celite and the filtrate concentrated in vacuo. The crude product was chromatographed on silica gel (10-50% EtOAc/hexanes) to give 10.2 g (93%) of D-pyroglytamic acid methyl ester as a colorless oil. 1H NMR (CDCl₃, 300 MHz) δ 6.95 (1H, s), 4.35 (1H, dd), 3.85 (3H, s), 2.70-2.30 (4H, m).

Step 2

To a solution of D-pyroglytamic acid methyl ester (10.2 g, 71.2 mmol) in 240 mL of Et₃N/CH₃CN (3:1) was added DMAP (0.91 g, 7.4 mmol, 0.1 eq) followed by di-tert-butyl dicarbonate (31.7 g, 145 mmol, 2 eq). After stirring at room temperature for 3 hr, the reaction mixture was diluted with EtOAc (730 mL), washed with 3% HCl, saturated NaHCO₃, and brine, dried (Na₂SO₄), and concentrated in vacuo. The crude product was purified using silica gel chromatography (10-30% EtOAc/hexanes) to give 11.6 g (67%) of N-Boc-D-pyroglutamic acid methyl ester as a yellowish solid. 1H NMR (CDCl₃, 300 MHz) δ 4.73 (1H, dd), 3.90 (3H, s), 2.80-2.20 (4H, m), 1.62 (9H, s).

Step 3

To a mixture of 1-bromo-3-fluorobenzene (0.79 g, 4.5 mmol) and magnesium turnings (0.12 g, 5.0 mmol) in anhydrous THF (8 mL) was added a small piece of iodine. The mixture was heated at reflux for 2 hr and no magnesium turnings left. The solution was cooled to 0° C. and transferred into a stirred solution of N-Boc-D-pyroglutamic acid methyl ester (0.80 g, 3.3 mmol) in anhydrous THF (4 mL) at −40° C. under argon atmosphere. After stirring at −40° C. for 1 hr and then at 0° C. for 1 hr, the reaction was quenched with 8 mL of 1:1 HOAc/MeOH and the mixture diluted with Et₂O (40 mL). The organic layers were washed with water and brine, dried (Na₂SO₄) and concentrated in vacuo. Purification by silica gel chromatography (10-20% EtOAc/hexanes) gave 0.57 g (51%) of (2R)-2-tert-butoxycarbonylamino-5-(3-fluorophenyl)-5-oxo-pentanoic acid methyl ester as a white solid. 1H NMR (CDCl₃, 300 MHz) δ 7.82 (1H, d), 7.75 (1H, d), 7.56 (1H, q), 7.38 (1H, t), 5.30 (1H, br.d), 4.50 (1H, br.s), 3.87 (3H, s), 3.20 (2H, m), 2.45 (1H, m), 2.20 (1H, m), 1.54 (9H, s).

Step 4

To a solution of (2R)-2-tert-butoxycarbonylamino-5-(3-fluoro-phenyl)-5-oxo-pentanoic acid methyl ester (0.57 g, 1.7 mmol) in 1.7 mL of CH₂Cl₂ at 0° C. was added dropwise 3.8 mL (49 mmol, 29 eq) of TFA. The mixture was stirred at 0° C. for 2 hr. After concentration in vacuo, the residue was dissolved in CH₂Cl₂, (30 mL), washed with 10% NaHCO₃, water, and brine, dried (Na₂SO₄) and concentrated in vacuo to give (2R)-5-(3-fluoro-phenyl)-3,4-dihydro-2H-pyrrole-2-carboxylic acid methyl ester as a colorless oil. 1H NMR (CDCl₃, 300 MHz) δ 7.77-7.70 (2H, m), 7.50 (1H, q), 7.27 (1H, tq), 5.05 (1H, tt), 3.90 (3H, s), 3.30-3.00 (2H, m), 2.55-2.30 (2H, m).

The oil was dissolved in 5 mL of absolute EtOH, and PtO₂ (5 mg, catalyst) was added. The mixture was hydrogenated at room temperature under a H₂ balloon for 16 hr. After filtration through Celite, the filtrate was concentrated. Chromatography on silica gel (10-20% EtOAc/hexanes) gave 0.33 g (87%) of pure (2R,5S)-5-(3-fluorophenyl)-pyrrolidine-2-carboxylic acid methyl ester as a color less oil. 1H NMR (CDCl₃, 300 MHz) δ 7.43-7.27 (3H, m), 7.04 (1H, t), 4.33 (1H, q), 4.05 (1H, q), 3.90 (3H, s), 2.43 (1H, br.s), 2.38-2.17 (3H, m), 1.80 (1H, m).

Step 5

(2R,5S)-5-(3-fluoro-phenyl)-pyrrolidine-2-carboxylic acid methyl ester (0.33 g, 1.5 mmol) was dissolved in 1,2-dichloroethane (2 mL) and Et₃N (1.0 mL, 7.4 mmol, 5 eq) was added followed by 4-chlorobenzenesulfonyl chloride (0.78 g, 3.7 mmol, 2.5 eq). The mixture was heated at 100° C. for 16 hr. Solvent was removed in vacuo and the residue was dissolved in EtOAc (10 mL), washed with 10% NaHCO₃ and brine, dried (Na₂SO₄) and concentrated in vacuo. Chromatography on silica gel (5-10% EtOAc/hexanes) yielded 0.45 g (75%) of pure (2R,5S)-1-(4-chloro-benzenesulfonyl)-5-(3-fluoro-phenyl)-pyrrolidine-2-carboxylic acid methyl ester as yellowish solid. 1H NMR (CDCl₃, 300 MHz) δ 7.67 (2H, d), 7.42 (2H, d), 7.24 (3H, m), 6.98 (1H, m), 4.95 (1H, t), 4.82 (1H, q), 3.94 (3H, s), 2.45-2.25 (3H, m), 2.05 (1H, m).

Step 6

To a solution of (2R,5S)-1-(4-chloro-benzenesulfonyl)-5-(3-fluoro-phenyl)-pyrrolidine-2-carboxylic acid methyl ester (0.45 g, 1.1 mmol) in anhydrous toluene (5 mL) at 0° C. was added dropwise diisobutylaluminum hydride (1M in hexane, 6.4 mL, 6.4 mmol). The mixture was allowed to warm to room temperature and stirred for 16 hr. The reaction was quenched by adding 1N HCl (10 mL) and the mixture diluted with EtOAc (20 mL). The organic phase was separated, washed with 10% NaHCO₃ and brine, dried (Na₂SO₄) and concentrated in vacuo to give 0.40 g (99%) of (2R,5S)-[1-(4-chloro-benzenesulfonyl)-5-(3-fluoro-phenyl)-pyrrolidin-2-yl]-methanol as a yellowish solid. 1H NMR (CDCl₃, 300 MHz) δ7.89 (2H, d), 7.62 (2H, d), 7.50-7.20 (3H, m), 7.06 (1H, td), 4.84 (1H, t), 4.05-3.85 (3H, m), 2.05 (2H, q), 1.95-1.80 (3H, m). The 1H NMR showed complete conversion of ester to alcohol and the material was used without further purification.

Step 7

To a solution of (2R,5S)-[1-(4-chloro-benzenesulfonyl)-5-(3-fluoro-phenyl)-pyrrolidin-2-yl]-methanol (0.027 g, 0.073 mmol) in 0.5 mL of CH₂Cl₂ was added 0.030 mL (0.22 mmol, 3 eq) of Et₃N followed by 0.015 g (0.074 mmol, 1 eq) of 4-nitrophenyl chloroformate. The mixture was stirred at room temperature for 16 hr. 4-Piperidinopiperidine (0.024 g, 0.14 mmol, 2 eq) was added and stirring continued for 16 hr. The mixture was diluted with 5 mL of CH₂Cl₂ and washed with 1N NaOH (2 mL×2), water (2 mL×2), and saturated brine (2 mL). The organic layer was dried (Na₂SO₄) and concentrated in vacuo to give a yellow gum. Purification using reverse phase prep-HPLC gave 0.030 g (73%) of the desired product (2R,5S)-[1,4′]bipiperidinyl-1′-carboxylic acid 1-(4-chloro-benzenesulfonyl)-5-(3-fluoro-phenyl)-pyrrolidin-2-ylmethyl ester as a white solid. 1H NMR (CD₃OD, 300 MHz) δ 7.98 (2H, d), 7.76 (2H, d), 7.46 (1H, q), 7.32 (2H, m), 7.10 (1H, t), 4.80 (1H, t), 4.50-4.35 (3H, m), 3.75-3.50 (4H, m), 3.00-2.95 (4H, m), 2.50-1.55 (14H, m). MS(ESI): MH⁺=564.1

Following procedures similar to those in Example 177, the compounds in Table 25 were prepared.

TABLE 24

| | | Exact MS, | MS ESI) MH⁺ |
|---|---|---|---|
| Compound | Structure | calc. | found |
| 177-A | 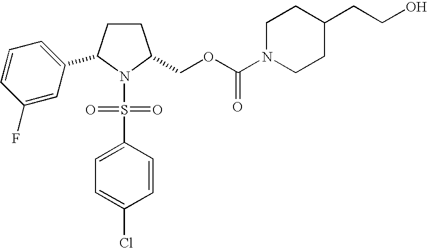 | 524.15 | 524.9 |
| 177-B | 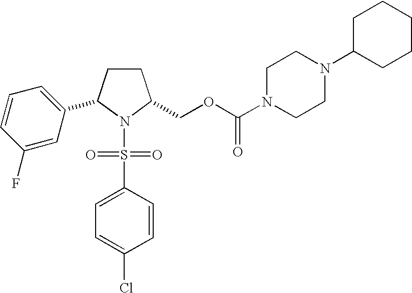 | 563.20 | 564.1 |
| 177-C | 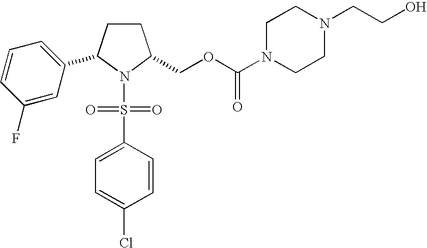 | 525.15 | 526.0 |
| 177-D | 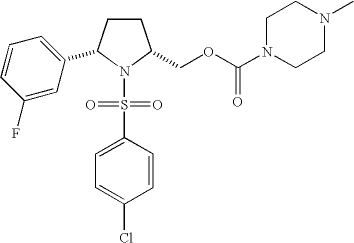 | 495.14 | 496.0 |
| 177-E | 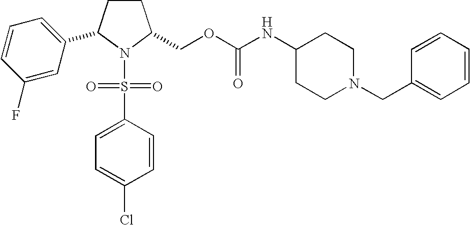 | 585.19 | 586.1 |
| 177-F | 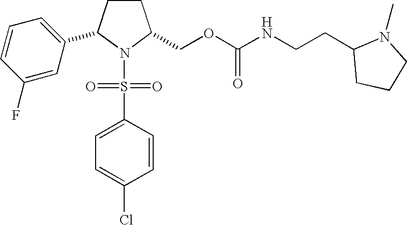 | 523.17 | 524.1 |
| 177-G | 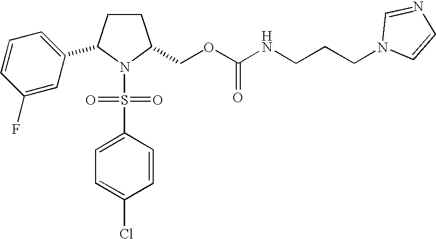 | 520.13 | 521.0 |
| 177-H | 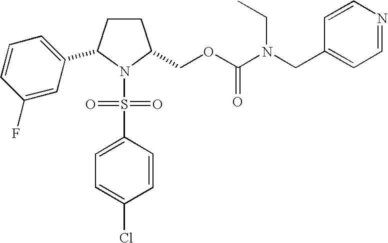 | 531.14 | 532.1 |

Assay:

Gamma secretase activity was determined as described by Zhang et al. (Biochemistry, 40 (16), 5049-5055, 2001). Activity is expressed either as a percent inhibition or as the concentration of compound producing 50% inhibition of enzyme activity.

Reagents

Antibodies W02, G2-10, and G2-11 were obtained from Dr. Konrad Beyreuther (University of Heidelberg, Heidelberg, Germany). W02 recognizes residues 5-8 of Aβ peptide, while G2-10 and G2-11 recognize the specific C-terminal structure of Aβ 40 and Aδ 42, respectively. Biotin-4G8 was purchased from Senetec (St. Louis, Mo.). All tissue culture reagents used in this work were from Life Technologies, Inc., unless otherwise specified. Pepstatin A was purchased from Roche Molecular Biochemicals; DFK167 was from Enzyme Systems Products (Livermore, Calif.).

cDNA Constructs, Tissue Culture, and Cell Line Construction

The construct SPC99-Lon, which contains the first 18 residues and the C-terminal 99 amino acids of APP carrying the London mutation, has been described (Zhang, L., Song, L., and Parker, E. (1999) J. Biol. Chem. 274, 8966-8972). Upon insertion into the membrane, the 17 amino acid signal peptide is processed, leaving an additional leucine at the N-terminus of Aβ. SPC99-Ion was cloned into the pcDNA4/TO vector (Invitrogen) and transfected into 293 cells stably transfected with pcDNA6/TR, which is provided in the T-REx system (Invitrogen). The transfected cells were selected in Dulbecco's modified Eagle's media (DMEM) supplemented with 10% fetal bovine serum, 100 units/mL penicillin, 100 g/mL streptomycin, 250 g/mL zeocin, and 5 g/mL blasticidin (Invitrogen). Colonies were screened for Aδ production by inducing C99 expression with 0.1 g/mL tetracycline for 16-20 h and analyzing conditioned media with a sandwich immunoassay (see below). One of the clones, designated as pTRE.15, was used in these studies.

Membrane Preparation

C99 expression in cells was induced with 0.1 g/mL tetracycline for 20 h. The cells were pretreated with 1 M phorbol 12-myristate 13-acetate (PMA) and 1 M brefeldin A (BFA) for 5-6 h at 37 C before harvesting. The cells were washed 3 times with cold phosphate-buffered saline (PBS) and harvested in buffer A containing 20 mM Hepes (pH 7.5), 250 mM sucrose, 50 mM KCl, 2 mM EDTA, 2 mM EGTA, and Complete protease inhibitor tablets (Roche Molecular Biochemicals). The cell pellets were flash-frozen in liquid nitrogen and stored at −70 C before use.

To make membranes, the cells were resuspended in buffer A and lysed in a nitrogen bomb at 600 psi. The cell lysate was centrifuged at 1500 g for 10 min to remove nuclei and large cell debris. The supernatant was centrifuged at 10000 g for 1 h. The membrane pellet was resuspended in buffer A plus 0.5 M NaCl, and the membranes were collected by centrifugation at 200000 g for 1 h. The salt-washed membrane pellet was washed again in buffer A and centrifuged at 100000 g for 1 h. The final membrane pellet was resuspended in a small volume of buffer A using a Teflon-glass homogenizer. The protein concentration was determined, and membrane aliquots were flash-frozen in liquid nitrogen and stored at −70 C.

γ-Secretase Reaction and Aβ Analysis

To measure γ-secretase activity, membranes were incubated at 37 C for 1 h in 50 L of buffer containing 20 mM Hepes (pH 7.0) and 2 mM EDTA. At the end of the incubation, Aβ 40 and Aβ 42 were measured using an electrochemiluminescence (ECL)-based immunoassay. Aβ 40 was identified with antibody pairs TAG-G2-10 and biotin-W02, while Aβ 42 was identified with TAG-G2-11 and biotin-4G8. The ECL signal was measured using an ECL-M8 instrument (IGEN International, Inc.) according to the manufacturer's instructions. The data presented were the means of the duplicate or triplicate measurements in each experiment. The characteristics of γ-secretase activity described were confirmed using more than five independent membrane preparations.

Using the above assay, the compounds of Examples 1-29, 31-33, 35-48, 50-61, 63-67,67A-67BR, 68,69, 71-74, 74A, 74B, 74C, 75, 76, 78-83, 85-99,101-159, 159A,159B, 159C,160, 160A-160AA, 161, 161A-161G, 162, 162A, 162B, 162C, 164, 164A, 164B, 164C, 165-167, 167A, 167B, 167C, 168, 168A, 169, 169A-169D, 170, 170A-170AD, 171-173, 173A-173T, and 174 showed IC₅₀ within the range of about 0.0002 to about 15 μM. The compounds of Examples 67B, 67E, 67N, 67P, 67U, 67AG, 67AT, 67AW, 67AY, 67BA, 67BD, 67BE, 67BG, 67BH, 67BL, 160B, 160K, 161, 161A, 161E, 161F, 173, 173A, 173B, 173C, 173E, 173G,173I, 173J, 173K, 173L and 173N showed IC₅₀ within the range of about 0.0002 to about 0.015 μM.

The γ-secretase inhibitory activity of some of the inventive compounds are shown below:

| Example | IC50 (μM) |
|---|---|
| 7-B | .0027 |
| 7-AT | .0038 |
| 7-BG | .0023 |
| 61-A | .0028 |
| 73 | .0002 |
| 73-A | .0007 |
| 73-C | .0018 |
| 73-E | .0027 |
| 73-J | .0008 |
| 73-N | .0024 |

Pharmaceutical compositions can comprise one or more of the compounds of formula I. For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active compound. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in a unit dosage form. In such form, the preparation is subdivided into suitably sized unit doses containing appropriate quantities of the active compound, e.g., an effective amount to achieve the desired purpose.

The quantity of active compound in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in one to four divided doses.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A compound of the formula 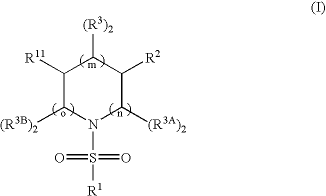
or a pharmaceutically acceptable salt, solvate or ester thereof, wherein:
(A) R¹ is selected from the group consisting of:
(1) unsubstituted aryl;
(2) aryl substituted with one or more R⁵ groups;
(3) unsubstituted heteroaryl; and
(4) heteroaryl substituted with one or more R⁵ groups,
(B) R² is selected from the group consisting of:
(1) alkyl;
(2) —XC(O)Y;
(3) —(C₁-C₆)alkylene-XC(O)Y;
(4) —(C₀-C₆)alkylene-(C₃-C₆)cycloalkylene-(C₀-C₆)alkylene-XC(O)Y;
(5) aryl;
(6) aryl substituted with one or more R⁵ groups;
(7) heteroaryl;
(8) heteroaryl substituted with one or more R⁵ groups;
(9) cycloalkylene-X-C(O)—Y;
(10) —CH₂—X—C(O)—NR³—Y;
(11) —CH₂—X—C(O)—Y; and
(12) —CH₂—X—C(O)—NR³—Y,
(C) Each R³ is independently selected from the group consisting of:
(1) H; and
(2) alkyl,
(D) Each R^{3A} and R^{3B} is independently selected from the group consisting of:
(1) H; and
(2) alkyl;
(E) R⁵ is independently selected from the group consisting of:
(1) halo;
(2) —CF₃;
(3) —OH;
(4) —O-alkyl;
(5) —OCF₃;
(6) —CN;
(7) —NH₂;
(8) —C(O)₂alkyl;
(9) —C(O)NR⁶R⁷;
(10) -alkylene-NR⁶R⁷;
(11) —NR⁶C(O)alkyl;
(12) —NR⁶C(O)aryl;
(13) —NR⁶C(O)heteroaryl; and
(14) —NR⁶C(O)NR⁶R⁷;
(F) X is selected from the group consisting of:
(1) —O—;
(2) —NH—;
(3) —N-alkyl; and
(4) —O-alkylene;
(G) Y is selected from the group consisting of:
(1) —NR⁶R⁷;
(2) —N(R³)(CH₂)_{b}NR⁶R⁷ wherein b is 2-6;
(3) unsubstituted aryl;
(4) unsubstituted heteroaryl;
(5) -alkyl;
(6) -cycloalkyl,
(7) unsubstituted arylalkyl;
(8) unsubstituted arylcycloalkyl;
(9) unsubstituted heteroarylalkyl;
(10) unsubstituted heteroarylcycloalkyl;
(11) unsubstituted arylheterocycloalkyl;
(12) substituted aryl;
(13) substituted heteroaryl;
(14) substituted arylalkyl;
(15) substituted arylcycloalkyl;
(16) substituted heteroarylalkyl;
(17) substituted heteroarylcycloalkyl; and
(18) substituted arylheterocycloalkyl;
(19) substituted heterocycloalkyl alkyl;
(20) unsubstituted heteroaryl alkyl;
(21) unsubstituted aryl alkyl heterocycloalkyl;
(22) unsubstituted heterocycloalkyl; and
(23) unsubstituted cycloalkyl,
wherein the aryl moiety in said substituted groups (12), (14), (15), (18), and (21) of said Y group, and the heteroaryl moiety in said substituted groups (13), (16), (17) and (20) of said Y group, are substituted with one or more substituents independently selected from the group consisting of:
(a) halo;
(b) —CF₃;
(c) —OH;
(d) —O-alkyl;
(e) —OCF₃;
(f) —CN;
(g) —NH₂;
(h) —C(O)₂(C₁-C₆)alkyl;
(i) —C(O)NR⁶R⁷;
(j) —(C₁-C₆)alkylene-NR⁶R⁷;
(k) —NR⁶C(O)alkyl;
(l) —NR⁶C(O)aryl;
(m) —NR⁶C(O)heteroaryl;
(n) —NR⁶C(O)NR⁶R⁷; and
(o) alkyl,
or Y is selected from the group consisting of: 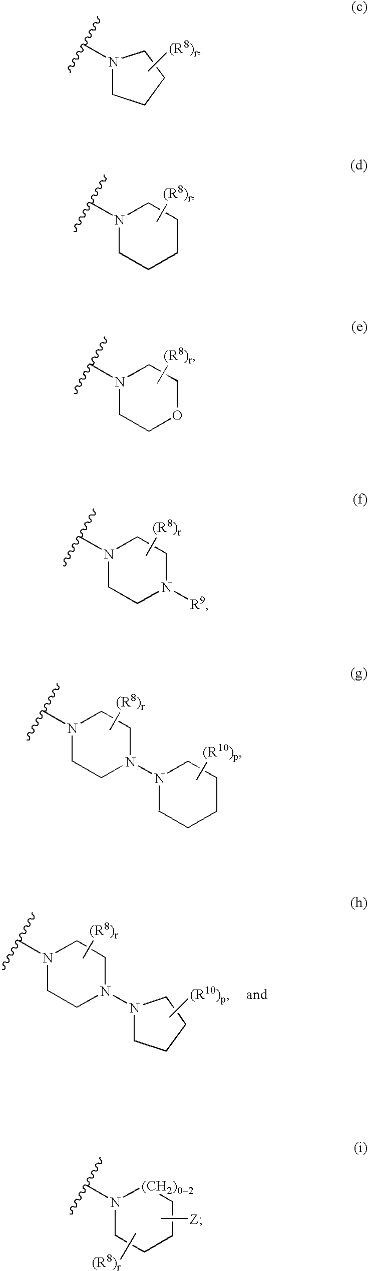
(H) R⁶ and R⁷ are independently selected from the group consisting of:
(1) H;
(2) alkyl;
(3) cycloalkyl;
(4) arylalkyl;
(5) heteroarylalkyl;
(6)
(7) 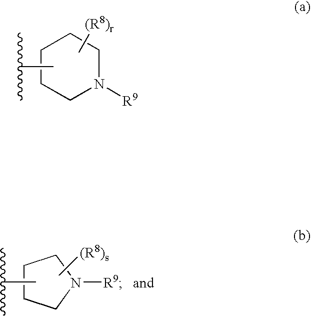
(8) heterocycloalkyl,
(I) Each R⁸ is independently selected from the group consisting of:
(1) alkyl;
(2) alkyl substituted with 1 to 4 hydroxy groups; and
(3) —OH,
(J) Each R⁹ is independently selected from the group consisting of:
(1) H;
(2) alkyl;
(3) alkyl substituted with 1 to 4 hydroxy groups;
(4) cycloalkyl;
(5) cycloalkyl substituted with 1 to 4 hydroxy groups;
(6) arylalkyl;
(7) heteroarylalkyl;
(8) —C(O)O-alkyl;
(9) alkylene-O-alkylene-OH;
(10) aryl substituted with one or more R⁵ groups;
(11) heteroaryl substituted with one or more R⁵ groups;
(12) unsubstituted heteroaryl;
(13) unsubstituted aryl;
(14) -alkylene-C(O)O-alkyl; and
(15) hydroxyalkyl-O-alkyl,
(K) Each R¹⁰ is independently selected from the group consisting of:
(1) H; and
(2) alkyl,
(L) R¹¹ is selected from the group consisting of:
(1) unsubstituted aryl;
(2) substituted aryl;
(3) unsubstituted heteroaryl,
(4) alkyl;
(5) cycloalkyl;
(6) unsubstituted arylalkyl;
(7) unsubstituted arylcycloalkyl,
(8) unsubstituted heteroarylalkyl;
(9) unsubstituted heteroarylcycloalkyl;
(10) unsubstituted arylheterocycloalkyl;
(11) alkoxyalkyl;
(12) substituted heteroaryl;
(13) substituted arylalkyl;
(14) substituted arylcycloalkyl;
(15) substituted heteroarylalkyl; and
(16) substituted arylheterocycloalkyl,
wherein the aryl moiety in said substituted groups (2), (13), (14) and (16) of said R¹¹ group, and the heteroaryl moiety in said substituted groups (12) and (15) of said R¹¹ group, are substituted with one or more substituents independently selected from the group consisting of:
(a) halo;
(b) —CF₃;
(c) —OH;
(d) —O-alkyl;
(e) —OCF₃;
(f) —CN;
(g) —NH₂;
(h) —C(O)₂(C₁-C₆)alkyl;
(i) —C(O)NR⁶R⁷;
(j) —(C₁-C₆)alkylene-NR⁶R⁷;
(k) —NR⁶C(O)alkyl;
(l) —NR⁶C(O)aryl;
(m) —NR⁶C(O)heteroaryl; and
(n) —NR⁶C(O)NR⁶R⁷;
(M) (1) m is 0 to 3, and if m is greater than 1, m moieties can be the same or different from one another;
(2) n is 0 to 3, and if n is greater than 1, n moieties can be the same or different from one another;
(3) o is 0 to 3, and if o is greater than 1, o moieties can be the same or different from one another;
such that m+n+o is 1, 2, 3 or 4,
(N) p is 0 to 4, and if greater than 1, p moieties can be the same or different from one another;
(O) r is 0 to 4, and if greater than 1, r moieties can be the same or different from one another;
(P) s is 0 to 3, and if greater than 1, s moieties can be the same or different from one another; and
(Q) Z is selected from the group consisting of:
(1) unsubstituted heterocycloalkyl;
(2) substituted heterocycloalkyl;
(3) —NH₂;
(4) —NH(alkyl);
(5) —N(alkyl)₂ wherein each alkyl is the same or different;
(6) —NH(unsubstituted cycloalkyl);
(7) —NH(substituted cycloalkyl);
(8) —N(alkyl)(unsubstituted cycloalkyl);
(9) —N(alkyl)(substituted cycloalkyl);
(10) —NH(unsubstituted aralkyl);
(11) —NH(substituted aralkyl);
(12) —N(alkyl)(aralkyl);
(13) —NH(unsubstituted heterocycloalkyl);
(14) —NH(substituted heterocycloalkyl);
(15) —N(alkyl)(unsubstituted heterocycloalkyl),
(16) —N(alkyl)(substituted heterocycloalkyl);
(17) —NH(unsubstituted heteroaralkyl);
(18) —NH(substituted heteroaralkyl);
(19) —NH-alkylene-(unsubstituted cycloalkyl);
(20) —NH-alkylene-(substituted cycloalkyl);
(21) —N(alkyl)alkylene-(unsubstituted cycloalkyl);
(22) —N(alkyl)alkylene-(substituted cycloalkyl);
(23) —NHalkylene-(unsubstituted heterocycloalkyl);
(24) —NHalkylene-(substituted heterocycloalkyl);
(25) —N(alkyl)alkylene-(unsubstituted heterocycloalkyl);
(26) —N(alkyl)alkylene-(substituted heterocycloalkyl);
(27) unsubstituted benzofused heterocycloalkyl; and
(28) substituted benzofused heterocycloalkyl;
(29) H; and
(30) —N(hydroxyalkyl)₂, wherein each alkyl may be the same or different,
wherein said substituted heterocycloalkyl moiety of substituents (2), (14), (16), (24), (26) and (27) of group Z, and said substituted cycloalkyl moiety of substituents (7), (9), (20) and (22) of group Z, and said substituted aryl moiety of substituent (11) of group Z, and said substituted heteroaryl moiety of substituent (18) of group Z, are substituted with 1 to 3 groups independently selected from the group consisting of:
(a) alkyl;
(b) —OH;
(c) —Oalkyl;
(d) —OC(O)alkyl;
(e) —OC(O)aryl;
(f) —NH₂;
(g) —NH(alkyl);
(h) —N(alkyl)₂ wherein each alkyl is the same or different;
(i) —NHC(O)alkyl;
(j) —N(alkyl)C(O)alkyl;
(k) —NHC(O)aryl;
(l) —N(alkyl)C(O)aryl;
(m) —C(O)alkyl;
(n) —C(O)aryl;
(o) —C(O)NH₂;
(p) —C(O)NH(alkyl);
(q) —C(O)N(alkyl)₂ wherein each alkyl is the same or different;
(r) —C(O)₂alkyl;
(s) -alkylene-C(O)Oalkyl;
(t) piperidinyl;
(u) pyrrolidinyl;
(v) 1,1-ethylenedioxy;
(w) aryl;
(x) heteroaryl; and
(y) —O—CH₂CH₂—O-wherein both oxygen atoms are bound to the same carbon atom, and provided that the aryl and heteroaryl moieties of said Z group are not substituted with said —O—CH₂CH₂—O-group.

2. The compound of claim 1 wherein:
(A) R¹ is aryl substituted with one or more R⁵ groups;
(B) n is 0 or 1 and m is 1, 2 or 3 such that m+n is 3;
(C) p is 0 or 1; and
(D) R² is —XC(O)Y, —(C₁-C₆)alkylene-XC(O)Y or —(C₀-C₆)alkylene-(C₃-C₆)cycloalkylene-(C₀-C₆)alkylene-XC(O)Y.

3. The compound of claim 2 wherein:
(A) R¹ is phenyl substituted with one or more R⁵ groups; and
(B) n is 0 and m is 3.

4. The compound of claim 1, wherein R² is 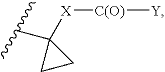
wherein X and Y are as defined.

5. The compound of claim 3 wherein R¹ is phenyl substituted with one or more halo atoms.

6. The compound of claim 1 wherein:
(A) R¹ is aryl substituted with one or more R⁵ groups;
(B) n is 0 or 1 and m is 1, 2 or 3 such that m+n is 3;
(C) p is 0 or 1;
(D) R² is —XC(O)Y, —(C₁-C₆)alkylene-XC(O)Y or —(C₀-C₆)alkylene-(C₃-C₆)cycloalkylene-(C₀-C₆)alkylene-XC(O)Y;
(E) X is O;
(F) Y is —NR⁶R⁷; or Y is selected from the group consisting of: 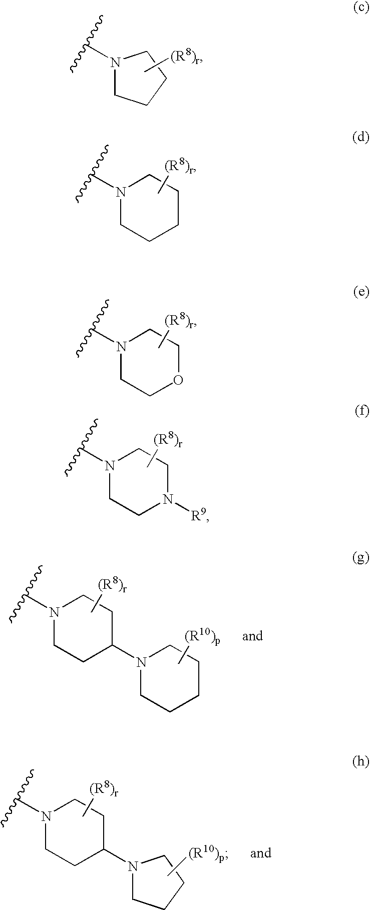
(G) R⁶ and R⁷ are independently selected from the group consisting of: H, methyl, ethyl, —(C₃-C₈)cycloalkyl, -aryl(C₁-C₆)alkyl, 4-pyridylmethyl, and

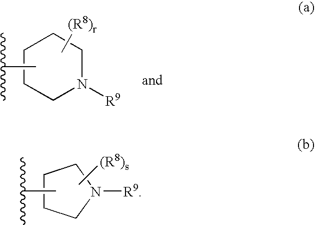

7. The compound of claim 6 wherein:
(A) R¹ is phenyl substituted with one or more R⁵ groups;
(B) n is 0 and m is 3;
(C) said group 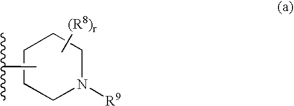
is a group of the formula: 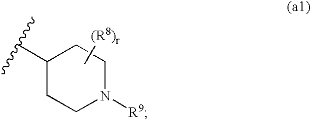
(D) said group 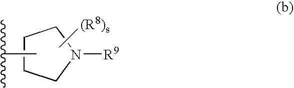
is a group of the formula: 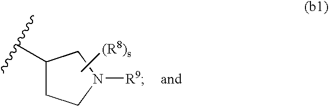
(E) R¹¹ is selected from the group consisting of: —(C₁-C₆)alkyl, (C₃-C₈)-cycloalkyl, aryl, aryl(C₁-C₆)alkyl and —(C₁-C₆)alkoxyalkyl.

8. The compound of claim 7 wherein said R¹¹ is selected from the group consisting of: methyl, ethyl, cyclohexyl, phenyl, benzyl, —(CH₂)₂phenyl, and —CH₂OCH₃.

9. The compound of claim 7 wherein R¹ is phenyl substituted with one or more halo atoms.

10. The compound of claim 8 wherein R¹¹ is phenyl substituted with one or more halo atoms.

11. The compound of claim 6 wherein Y is selected from the group consisting of:

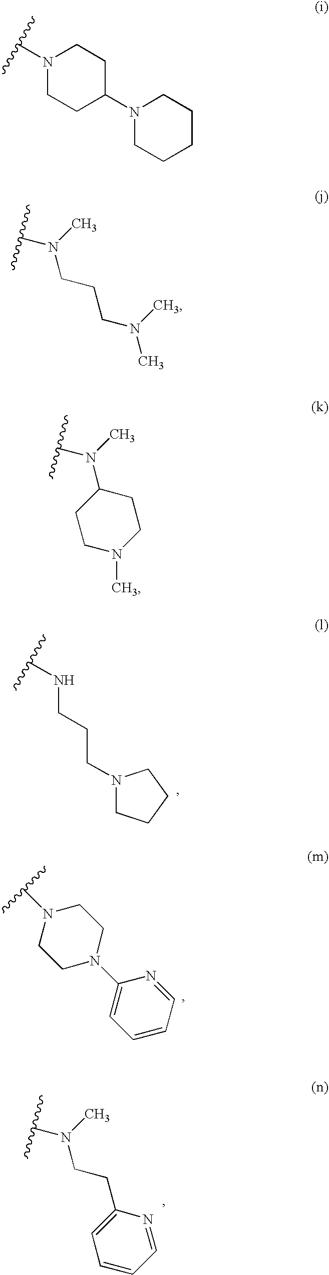

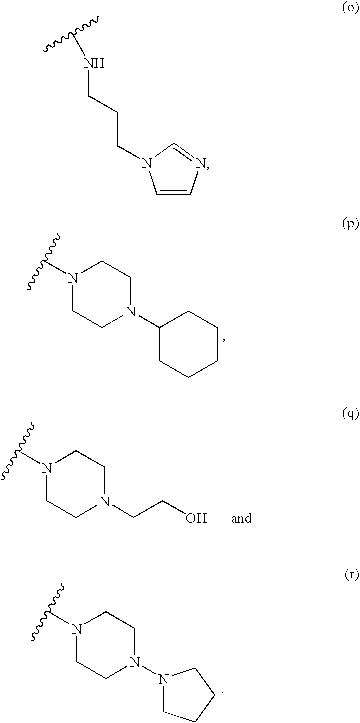

12. The compound of claim 1 selected from a final compound of Examples 1-29, 31-33, 35-48, 50-61, 63-67,67A-67BR, 68,69, 71-74, 74A, 74B, 74C, 75, 76, 78-83, 85-99,101-159,159A, 159B, 159C, 160, 160A-160AA, 161, 161A-161G, 162, 162A, 162B, 162C, 164, 164A, 164B, 164C, 165-167, 167A, 167B, 167C, 168, 168A, 169, 169A-169D, 170, 170A-170AD, 171-173, 173A-173T, and 174.

13. The compound of claim 1 selected from a final compound of Examples 67B, 67E, 67N, 67P, 67U, 67AG, 67AT, 67AW, 67AY, 67BA, 67BD, 67BE, 67BG, 67BH, 67BL, 160B, 160K, 161, 161A, 161E, 161F, 173, 173A, 173B, 173C, 173E, 173G, 173I, 173J, 173K, 173L and 173N.

14. The compound of claim 1 selected from a final compound of Examples 7-B, 7-AT, 7-BG, 61-A, 73, 73-A, 73-C, 73-E, 73-J, and 73-N.

15. A pharmaceutical composition comprising at least one compound of claim 1 and at least one pharmaceutically acceptable carrier.

16. A method of inhibiting gamma-secretase in a patient in need of such treatment comprising administering to said patient a therapeutically effective amount of one or more compounds of claim 1.

17. A method of treating one or more neurodegenerative diseases in a patient in need of such treatment comprising administering to said patient a therapeutically effective amount of one or more compounds of claim 1.

18. A method of inhibiting the deposition of beta amyloid protein in a patient in need of such treatment comprising administering to said patient a therapeutically effective amount of one more compounds of claim 1.

19. A method of treating Alzheimer's disease in a patient in need of such treatment comprising administering to said patient a therapeutically effective amount of one or more compounds of claim 1.

20. A compound selected from the group consisting of:

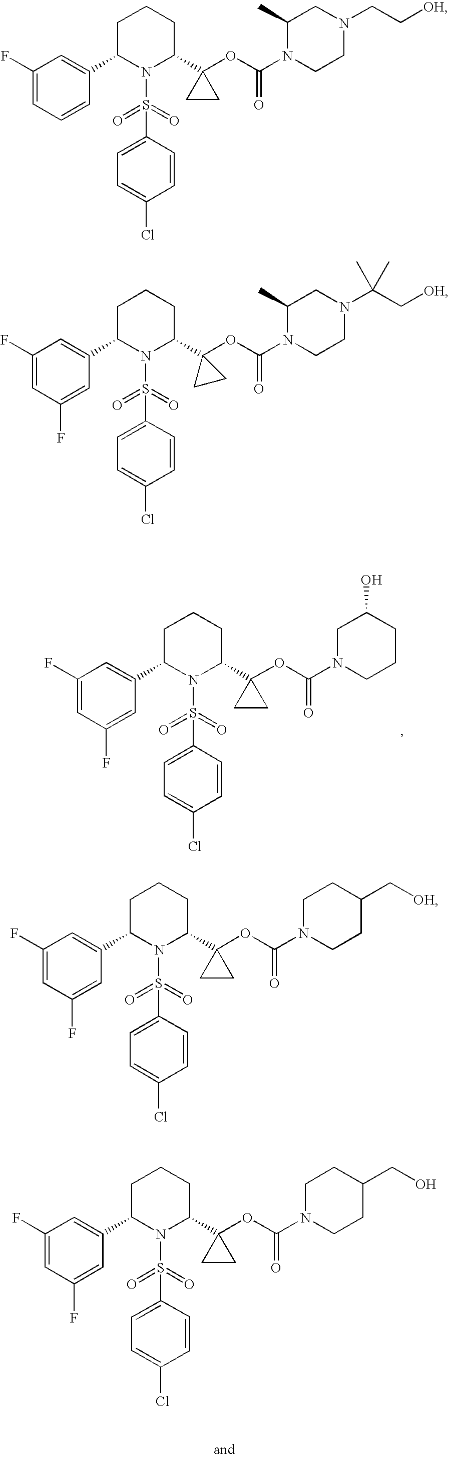

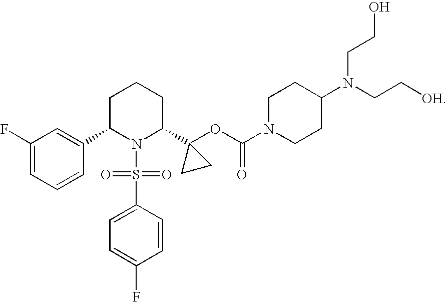

21. A pharmaceutical composition comprising a therapeutically effective amount of a compound of claim 20, or a pharmaceutically acceptable salt, ester or solvate thereof, together with a pharmaceutically acceptable excipient, diluent or carrier.

22. A method of inhibiting gamma-secretase in a patient in need of such treatment comprising administering to said patient a therapeutically effective amount of one or more compounds of claim 20.

23. A method of treating one or more neurodegenerative diseases in a patient in need of such treatment comprising administering to said patient a therapeutically effective amount of one or more compounds of claim 20.

24. A method of inhibiting the deposition of beta amyloid protein in a patient in need of such treatment comprising administering to said patient a therapeutically effective amount of one more compounds of claim 20.

25. A method of treating Alzheimer's disease in a patient in need of such treatment comprising administering to said patient a therapeutically effective amount of one or more compounds of claim 20.

26. A compound of the following formula 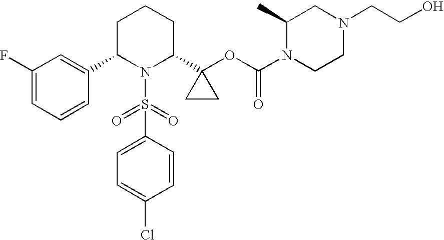
or a pharmaceutically acceptable salt, ester or solvate of said compound.

27. A compound of the following formula 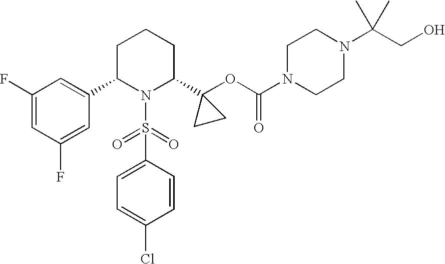
or a pharmaceutically acceptable salt, ester or solvate of said compound.

28. A compound of the following formula 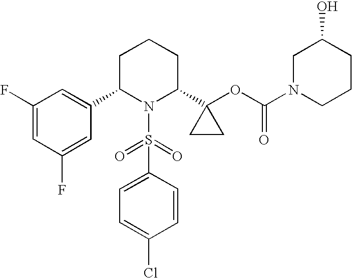
or a pharmaceutically acceptable salt, ester or solvate of said compound.

29. A compound of the following formula 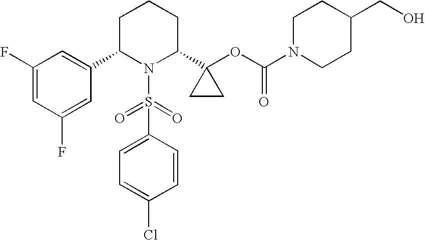
or a pharmaceutically acceptable salt, ester or solvate of said compound.

30. A compound of the following formula 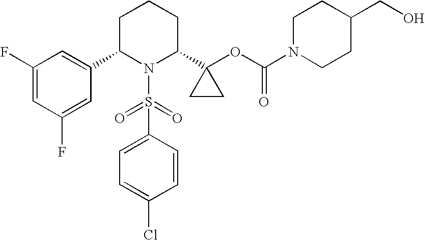
or a pharmaceutically acceptable salt, ester or solvate of said compound.

31. A compound of the following formula 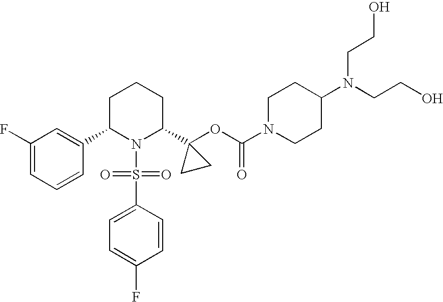
or a pharmaceutically acceptable salt, ester or solvate of said compound.
